(19) United States
(12) Patent Application Publication
(10) Pub. No.: US 2026/0284187 A1
(43) Pub. Date: Sep. 24, 2026

(54) POLYSACCHARIDE ADJUVANTS FOR USE IN PROTEIN-BASED OR RNA-BASED VACCINES

(71) Applicants: The Children's Medical Center Corporation, Boston, MA (US); The Brigham and Women's Hospital, Inc., Boston, MA (US)

(72) Inventors: Ivan Zanoni, Brookline, MA (US); Jeffrey M. Karp, Brookline, MA (US); Bryan Laulicht, New York, NY (US); Kautilya Jena, Boston, MA (US)

(73) Assignees: The Children's Medical Center Corporation, Boston, MA (US); The Brigham and Women's Hospital, Inc., Boston, MA (US)

(21) Appl. No.: 19/477,074

(22) PCT Filed: Apr. 19, 2024
(86) PCT No.: PCT/US2024/025574

§ 371 Date: Oct. 20, 2025

Related U.S. Application Data
(60) Provisional application No. 63/461,156, filed on Apr. 21, 2023.

(51) Int. Cl. A61K 39/39; A61K 39/00; A61K 39/215; A61K 39/245; A61K 39/29; A61P 37/04

(52) U.S. Cl. CPC A61K 39/39; A61K 39/0002; A61K 39/215; A61K 39/245; A61K 39/292; A61P 37/04; A61K 2039/53; A61K 2039/55505; A61K 2039/55583

(57) ABSTRACT

Provided herein are adjuvantation systems comprising mannan (e.g., a plant mannan or a fungal manna) for use with Beta coronavirus (e.g., MERS-CoV, SARS-CoV-1, or SARS-CoV-2) vaccines and immunogenic compositions comprising the adjuvantation system and a Beta coronavirus antigen or a nucleic acid encoding the Beta coronavirus antigen.

### RELATED APPLICATIONS

This application claims the benefit under 35 U.S.C. § 119(e) of U.S. provisional application No. 63/461,156, filed Apr. 21, 2023, which is incorporated by reference herein in its entirety.

### FEDERALLY SPONSORED RESEARCH

This invention was made with government support under Grant Number AI165505, awarded by the National Institutes of Health. The Government has certain rights in the invention.

### BACKGROUND

Adjuvants can be used for the enhancement of vaccine immunogenicity, broadening the response of the immunogen to other antigens, and/or increasing the stability of the formulation. This can lead to the need for fewer immunizations required for protective immunity and/or restoration in non- or less-responding individuals. However, the immunological benefit of adding adjuvants to vaccine formulations is not always replicated in clinical studies.

### SUMMARY

The most widely used adjuvants are aluminum salts, but the benefit of their use is not always present. Carbohydrate and carbohydrate-containing molecules, such as mannans, exert immunomodulatory effects on the innate immune system, activating a multitude of receptors and downstream pathways including CARD9-dependent pathways that can lead to distinct immune profiles and T cell proliferation. However, it is not clear whether activation of these cellular and molecular pathways confers clinical advantages over other non-polysaccharide molecules that activate different signaling pathways.

Accordingly, some aspects of the present disclosure provide an immunogenic composition comprising an adjuvantation system comprising a mannan and a nucleic acid encoding an antigen. In some embodiments, the mannan is a plant mannan or a fungal mannan. In some embodiments, the mannan is a glucomannan, a galactomannan, or a galactoglucomannan. In some embodiments, the mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan. In some embodiments, the plant mannan is a konjac glucomannan. In some embodiments, the plant mannan is from Ceratonia siliqua, Phytelephas macrocarpa, Schizolobium amazonicum, Schizolobium parahybum, Carum carvi, Cyamopsis tetragonolobus, Amorphophallus konjac, Coffea arabica, Aloe barbadensis, and/or Cesalpinia spinosa. In some embodiments, the fungal mannan is a yeast mannan. In some embodiments, the fungal mannan is Candida mannan or Saccharomyces mannan. In some embodiments, the fungal mannan is a Candida albicans mannan. In some embodiments, the fungal mannan is derived from Saccharomyces cerevisiae.

In some embodiments, the adjuvantation system further comprises an aluminum salt. In some embodiments, the aluminum salt is aluminum hydroxide, aluminum phosphate, or aluminum hydroxyphosphate. In some embodiments, the weight ratio of aluminum salt to mannan in the adjuvantation system is between 1:1 and 1:100. In some embodiments, the weight ratio of aluminum salt to mannan in the adjuvantation system is 1:1, 1:2, 1:3, 1:4, 1:5, 1:10, or 1:100.

In some embodiments, the nucleic acid is DNA or RNA. In some embodiments, the RNA is a messenger RNA (mRNA).

In some embodiments, the antigen is a bacterial antigen, a viral antigen, a parasitic antigen or a fungal antigen. In some embodiments, the viral antigen comprises a spike protein, a nucleocapsid protein, or a glycoprotein. In some embodiments, the viral antigen comprises a Beta coronavirus protein or polypeptide. In some embodiments, the Beta coronavirus protein or polypeptide comprises a Beta coronavirus spike protein or spike protein receptor binding domain (RBD). In some embodiments, the Beta coronavirus spike protein is a MERS-CoV spike protein, a SARS-CoV-1 spike protein, or a SARS-CoV-2 spike protein. In some embodiments, the Beta coronavirus spike protein RBD is a MERS-CoV spike protein RBD, a SARS-CoV-1 spike protein RBD, or a SARS-CoV-2 spike protein RBD. In some embodiments, the viral antigen comprises an antigen from human papillomavirus. In some embodiments, the antigen from human papillomavirus is selected from L1 antigen, E6 antigen, E7 antigen, or a combination thereof. In some embodiments, the viral antigen comprises an antigen from hepatitis B virus. In some embodiments, the antigen from hepatitis B virus is HBsAg. In some embodiments, the viral antigen comprises an antigen from Epstein-Barr virus. In some embodiments, the antigen from Epstein-Barr virus is Epstein-Barr nuclear antigen. In some embodiments, the viral antigen is SV40.

In some embodiments, the ratio of the nucleic acid to the adjuvantation system is between 1:1 and 1:100. In some embodiments, the ratio of the nucleic acid to the adjuvantation system is 1:1, 1:10, or 1:100. In some embodiments, the adjuvantation system and the nucleic acid are admixed.

In some aspects, the present disclosure provides an immunogenic composition comprising an adjuvantation system comprising a plant mannan; and an antigen. In some embodiments, the plant mannan is a glucomannan, a galactomannan, or a galactoglucomannan. In some embodiments, the plant mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan. In some embodiments, the plant mannan is a konjac glucomannan.

In some embodiments, the adjuvantation system further comprises an aluminum salt. In some embodiments, the aluminum salt is aluminum hydroxide, aluminum phosphate, or aluminum hydroxyphosphate. In some embodiments, the weight ratio of aluminum salt to plant mannan in the adjuvantation system is between 1:1 and 1:100. In some embodiments, the weight ratio of aluminum salt to plant mannan in the adjuvantation system is 1:1, 1:2, 1:3, 1:4, 1:5, 1:10, or 1:100.

In some embodiments, the antigen is a bacterial antigen, a viral antigen, or a fungal antigen. In some embodiments, the viral antigen comprises a Beta coronavirus protein or polypeptide. In some embodiments, the viral antigen comprises a nucleic acid encoding a Beta coronavirus protein or polypeptide. In some embodiments, the nucleic acid is DNA or RNA. In some embodiments, the RNA is a messenger RNA (mRNA).

In some embodiments, the Beta coronavirus protein or polypeptide comprises a Beta coronavirus spike protein or spike protein receptor binding domain (RBD). In some embodiments, the Beta coronavirus spike protein is a MERS-CoV spike protein, a SARS-CoV-1 spike protein, or a SARS-CoV-2 spike protein. In some embodiments, the Beta coronavirus spike protein RBD is a MERS-CoV spike protein RBD, a SARS-CoV-1 spike protein RBD, or a SARS-CoV-2 spike protein RBD.

In some embodiments, the adjuvantation system and the antigen are admixed.

In some embodiments, the present disclosure provides a vaccine comprising an immunogenic composition and a pharmaceutically acceptable excipient.

Accordingly, some aspects of the present disclosure provide an immunogenic composition comprising an adjuvantation system comprises (1) a mannan; and (2) a nucleic acid or a protein. In some embodiments, the nucleic acid is an antigen or encodes an antigen. In some embodiments, the composition is formulated in nanoparticle, liposome, emulsion, or a combination thereof. In some embodiments, the nucleic acid is outside the nanoparticle. In some embodiments, the nucleic acid in within the nanoparticle. In some embodiments, the nucleic acid is a DNA, an RNA or a combination thereof. In some embodiments, the mannan is within the nanoparticle. In some embodiments, the mannan is outside the nanoparticle. In some embodiments, the mannan comprises a linear polysaccharide or a branched polysaccharide. In some embodiments, the mannan is within lipid membrane of the lipid nanoparticle. In some embodiments, the liposomes comprising nucleic acid forms a stable formulation with the adjuvantation system, wherein the liposomes show minimal physicochemical interaction with the adjuvantation system. In some embodiments, the adjuvantation system comprises a complex of mannan and alum.

Other aspects of this disclosure provide, a method of stimulating immune cell recruitment in a subject in need thereof, the method comprising administering a composition comprising an adjuvantation system and a nucleic acid, wherein the adjuvantation system comprises a mannan. In some embodiments, the method recruits more immune cells relative to a subject treated with a corresponding comparable composition that does not comprise mannan.

Other aspects of this disclosure provide, a method of inducing an immune response against a pathogen in a subject in need thereof, the method comprising administering to the subject an adjuvantation system comprising a mannan, and a nucleic acid encoding an antigen. In some embodiments, the mannan is a plant mannan or a fungal mannan. In some embodiments, the mannan is a glucomannan, a galactomannan, or a galactoglucomannan. In some embodiments, the plant mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan. In some embodiments, the plant mannan is a konjac glucomannan. In some embodiments, the fungal mannan is a yeast mannan. In some embodiments, the fungal mannan is a Candida albicans mannan.

Other aspects of this disclosure provide, a method of inducing immune response by draining lymph node in a subject in need thereof, the method comprising administering a composition comprising an adjuvantation system and a nucleic acid in a subject in need thereof, wherein the adjuvantation system comprises a mannan. In some embodiments, the subject is a mammalian subject.

In some embodiments, the adjuvantation system further comprises an aluminum salt. In some embodiments, the aluminum salt is aluminum hydroxide, aluminum phosphate, or aluminum hydroxyphosphate. In some embodiments, the weight ratio of aluminum salt to mannan is in the adjuvantation system between 1:1 and 1:100. In some embodiments, the weight ratio of aluminum salt to mannan in the adjuvantation system is 1:1, 1:2, 1:3, 1:4, 1:5, 1:10, or 1:100.

In some embodiments, the nucleic acid is DNA or RNA. In some embodiments, the RNA is a messenger RNA (mRNA).

In some embodiments, the antigen is a bacterial antigen, a viral antigen, or a fungal antigen. In some embodiments, the viral antigen comprises a Beta coronavirus protein or polypeptide. In some embodiments, the Beta coronavirus protein or polypeptide comprises a Beta coronavirus spike protein or spike protein receptor binding domain (RBD). In some embodiments, the Beta coronavirus spike protein is a MERS-CoV spike protein, a SARS-CoV-1 spike protein, or a SARS-CoV-2 spike protein. In some embodiments, the Beta coronavirus spike protein RBD is a MERS-CoV spike protein RBD, a SARS-CoV-1 spike protein RBD, or a SARS-CoV-2 spike protein RBD.

In some embodiments, the ratio of the nucleic acid to the adjuvantation system is between 1:1 and 1:100. In some embodiments, the ratio of the nucleic acid to the adjuvantation system is 1:1, 1:10, or 1:100. In some embodiments, the adjuvantation system and the nucleic acid are admixed.

In some aspects, this disclosure provides a method of inducing an immune response against a pathogen in a subject in need thereof, the method comprising administering to the subject an adjuvantation system comprising a plant mannan, and an antigen. In some embodiments, the plant mannan is a glucomannan, a galactomannan, or a galactoglucomannan. In some embodiments, the plant mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan. In some embodiments, the plant mannan is a konjac glucomannan.

In some embodiments, the adjuvantation system further comprises an aluminum salt. In some embodiments, the aluminum salt is aluminum hydroxide, aluminum phosphate, or aluminum hydroxyphosphate. In some embodiments, the weight ratio of aluminum salt to plant mannan is between 1:1 and 1:100. In some embodiments, the weight ratio of aluminum salt to plant mannan in the adjuvantation system is 1:1, 1:2, 1:3, 1:4, 1:5, 1:10, or 1:100.

In some embodiments, the antigen is a bacterial antigen, a viral antigen, or a fungal antigen. In some embodiments, the viral antigen comprises a Beta coronavirus protein or polypeptide. In some embodiments, the viral antigen comprises a nucleic acid encoding a Beta coronavirus protein or polypeptide. In some embodiments, the nucleic acid is DNA or RNA. In some embodiments, the RNA is a messenger RNA (mRNA).

In some embodiments, the Beta coronavirus protein or polypeptide comprises a Beta coronavirus spike protein or spike protein receptor binding domain (RBD). In some embodiments, the Beta coronavirus spike protein is a MERS-CoV spike protein, a SARS-CoV-1 spike protein, or a SARS-CoV-2 spike protein. In some embodiments, the Beta coronavirus spike protein RBD is a MERS-CoV spike protein RBD, a SARS-CoV-1 spike protein RBD, or a SARS-CoV-2 spike protein RBD.

In some embodiments, the adjuvantation system and the antigen are admixed.

In some embodiments, the subject is a human. In some embodiments, the subject is a human neonate, a human infant, an adult human, or an elderly human. In some embodiments, the subject is a companion animal or a research animal. In some embodiments, the subject is immune-compromised, has chronic lung disease, asthma, cardiovascular disease, cancer, obesity, diabetes, chronic kidney disease, and/or liver disease.

In some embodiments, the administration is intramuscular administration, intradermal administration, oral administration, intravenous administration, topical administration, intranasal administration, or sublingual administration. In some embodiments, the administration is intramuscular administration. In some embodiments, the administration is prophylactic.

In some aspects, the present disclosure provides an adjuvantation system comprising a plant mannan for use in inducing an immune response against a pathogen in a subject in need thereof. In some embodiments, the mannan is a glucomannan, a galactomannan, or a galactoglucomannan. In some embodiments, the mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan. In some embodiments, the mannan is a konjac glucomannan.

In some embodiments, the adjuvantation system further comprises an aluminum salt. In some embodiments, the aluminum salt is aluminum hydroxide, aluminum phosphate, or aluminum hydroxyphosphate. In some embodiments, the weight ratio of aluminum salt to mannan in the adjuvantation system is between 1:1 and 1:100. In some embodiments, the weight ratio of aluminum salt to mannan in the adjuvantation system is 1:1, 1:2, 1:3, 1:4, 1:5, 1:10, or 1:100.

### BRIEF DESCRIPTION OF DRAWINGS

The accompanying drawings are not intended to be drawn to scale. For purposes of clarity, not every component may be labeled in every drawing. In the drawings:

FIGS. 1A-1G show intradermal injection of alum-mannan with intramuscular injection of Comirnaty (BNT162b2 mRNA, Pfizer-BioNTech) potentiates the neutralizing activity of sera 28 days post-priming. FIG. 1A is a schematic of a vaccine administration schedule where mice were injected intradermally (i.d.) or intramuscularly (i.m.) with Comirnaty (BNT162b2 mRNA, Pfizer-BioNTech) alone or with alum-mannan, or alum-mannan with a pre-fusion stabilized SARS-CoV-2 WA1 Spike trimer on day 0 and day 14. The data show IgG (FIG. 1B), IgG1 (FIG. 1C), and IgG2c (FIG. 1D) measured before priming (day 0), 14 days post-priming (day 14), or 14 days post-boosting (day 28) with Comirnaty (BNT162b2 mRNA, Pfizer-BioNTech; “Pfizer”), a protein antigen with alum-mannan (“Spike+AM”), or Comirnaty admixed with alum-mannan (“Pfizer+AM”). FIG. 1E shows neutralization titers for ancestral SARS-CoV-2 WA1 in mouse sera collected before priming (day 0), 14 days post-priming (day 14), or 14 days post-boosting (day 28). FIG. 1F shows the titer of neutralizing antibody against SARS-CoV-2 WA1 and SARS-CoV-2 B.1.1.529 in the sera of mice immunized with Comirnaty (BNT162b2 mRNA, Pfizer-BioNTech; “Pfizer”), a pre-fusion stabilized SARS-CoV-2 WA1 Spike trimer with alum-mannan (“Spike+AM”), or Comirnaty admixed with alum-mannan (“Pfizer+AM”) before priming (day 0), 14 days post-priming (day 14), or 14 days post-boosting (day 28). FIG. 1G shows the alum-mannan adjuvant does not change the levels of antibodies driven by a mRNA vaccine, as measured by mice administered Comirnaty (BNT162b2 mRNA, Pfizer-BioNTech; “Pfizer”), a pre-fusion stabilized SARS-CoV-2 WA1 Spike trimer with alum-mannan (“Spike+AM”), or Comirnaty admixed with alum-mannan (“Pfizer+AM)” before priming (day 0), 14 days post-priming (day 14), or 14 days post-boosting (day 28). ns=not significant; * p<0.05; ** p<0.01; p<0.001; **** p<0.0001. Dotted line parallel to the x-axis indicates lower of detection.

FIGS. 2A-2D show intramuscular injection of alum-mannan drives anti-Spike IgG induction. FIG. 2A is a schematic of a vaccine administration schedule where mice were injected intradermally (ID) or intramuscularly (IM) with a pre-fusion stabilized SARS-COV-2 WA1 Spike trimer (Spike) with mannan (SM) or with alum-mannan (SAM). IgG (FIG. 2B), IgG1 (FIG. 2C), and IgG2c (FIG. 2D) were measured in the sera of mice immunized with a protein antigen and mannan (SM) or with a protein antigen and alum-mannan (SAM) intradermally (ID) or intramuscularly (IM) before priming (day 0), 14 days post-priming (day 14), or 14 days post-boosting (day 28). ns=not significant; * p<0.05; ** p<0.01; *** p<0.001; **** p<0.0001. Dotted line parallel to the x-axis indicates lower of detection.

FIGS. 3A-3E show that lipid nanoparticles and lipid nanoparticles comprising mRNA are physically stable in the presence of excess alum-mannan complex using dynamic light scattering analysis. Alum (10 μg/ml) mixed with varying concentrations of mannan to form an alum-mannan complex with a weight ratio (alum:mannan) of 1:1 (FIG. 3A), 1:2 (FIG. 3B), 1:5 (FIG. 3C), or 1:10 (FIG. 3D) was incubated with 10 μg/ml of liposomal nanoparticle comprising an mRNA vaccine. The multimodal particle size distribution was evaluated 1 hour after admixing. Separation of peaks in all cases indicates that the mRNA vaccine lipid nanoparticles were intact and that minimal physicochemical interactions between the alum-mannan complex and the mRNA vaccine were observed. FIG. 3E shows that separation of peaks persisted up to 24 hours after admixing 1 μg/ml mRNA vaccine at a 1:1, 1:10, or 1:100 weight ratio (mRNA vaccine:alum-mannan) with 1:100 alum-mannan complex.

FIGS. 4A-4B show premature mRNA release is minimal when co-formulated with alum-mannan. FIG. 4A shows released mRNA of Comirnaty (BNT162b2 mRNA, Pfizer-BioNTech; “Pfizer vaccine”) alone or incubated with alum-mannan at a 1:1, 1:10, or 1:100 weight ratio (“Pfizer-vaccine:alum-mannan”) measured after 24 hours at 4° C. FIG. 4B shows that the mRNA vaccine Comirnaty remained relatively stable when formulated with or without alum-mannan, releasing only 3-4% of mRNA into solution.

FIGS. 5A-5K show that co-formulation of an mRNA vaccine with alum-mannan leads to superior protection against a viral antigen, as compared to the mRNA vaccine alone. FIG. 5A shows is a schematic of a vaccine administration schedule in which mice were administered Comirnaty (BNT162b2 mRNA, Pfizer-BioNTech) with or without alum-mannan. IgG (FIG. 5B), IgG1 (FIG. 5C), and IgG2c (FIG. 5D) levels were evaluated before priming (day 0), 14 days post-priming (day 14), or 14 days post-boosting (day 28) after intramuscular injection of Comirnaty (BNT162b2 mRNA, Pfizer-BioNTech; “Pfizer”) alone or admixed with alum-mannan (“Pfizer+AM”). FIG. 5E shows the titer of neutralizing antibody against SARS-CoV-2 WA1 in the sera of mice immunized with Comirnaty (BNT162b2 mRNA, Pfizer-BioNTech; “Pfizer”) or Comirnaty admixed with alum-mannan (“Pfizer+AM”) before priming (day 0), 14 days post-priming (day 14), 14 days post-boosting (day 28). FIG. 5F shows a comparison of the neutralizing antibody titers against SARS-CoV-2 WA1 and B.1.1.529 in the sera of mice immunized with Comirnaty (BNT162b2 mRNA, Pfizer-BioNTech) or Comirnaty admixed with alum-mannan. FIG. 5G shows a comparison of the neutralizing titers (EC₅₀) against SARS-CoV-2 WA1 and B.1.1.529 in the sera of mice immunized with Comirnaty (BNT162b2 mRNA, Pfizer-BioNTech) or Comirnaty admixed with alum-mannan. FIG. 5H shows a schematic of a vaccine schedule in which mice were administered saline or Comirnaty (BNT162b2 mRNA, Pfizer-BioNTech) with or without alum-mannan, and evaluated for 5 weeks post-initial immunization. FIG. 5I shows IgG levels evaluated before priming (day 0), 14 days post-priming (day 14), 14 days-post-boosting (day 28), or 4 weeks post-priming (day 35) after intramuscular injection of Comirnaty (“C”) or Comirnaty with alum-mannan (“CAM”). FIG. 5J shows neutralizing titers against SARS-CoV-2 WA1 or SARS-CoV-2 B.1.1.529 evaluated 14 days-post-boosting (day 28) or 4 weeks post-priming (day 35) after intramuscular injection of saline (“S”), Comirnaty (“C”), or Comirnaty with alum-mannan (“CAM”). FIG. 5K shows IgG titers decrease 1 month post-boosting (day 56) in mice receiving an intramuscular injection of Comirnaty (BNT162b2 mRNA, Pfizer-BioNTech; “Pfizer”) alone or admixed with alum-mannan (“Pfizer+AM”). However, IgG titers can be re-boosted following a third administration (day 56) of Comirnaty admixed with alum-mannan (“PAM”). ns=not significant; * p<0.05; ** p<0.01; *** p<0.001; **** p<0.0001. Dotted line parallel to the x-axis indicates lower of detection.

FIGS. 6A-6F show that co-formulation of an mRNA vaccine with alum-mannan enhances the production of cross-reactive antigen-specific antibodies targeting variants of the antigen. FIG. 6A shows a schematic of a vaccine administration schedule in which mice were administered saline, alum-mannan, Comirnaty (BNT162b2 mRNA, Pfizer-BioNTech), Comirnaty with alum, or Comirnaty with alum-mannan and evaluated up to 84 days post-initial immunization. IgG (FIG. 6B), IgG1, and IgG2c (FIG. 6C) levels were evaluated up to 84 days post-initial immunization with saline (“S”), alum-mannan (“M”), Comirnaty (BNT162b2 mRNA, Pfizer-BioNTech; “C”), Comirnaty with alum (“CA”), or Comirnaty with alum-mannan (“CAM”). FIG. 6D shows the percent of SARS-CoV-2 WA1 Spike protein-specific plasma cells present in the bone marrow of mice immunized with Comirnaty (BNT162b2 mRNA, Pfizer-BioNTech), Comirnaty with alum, or the FDC vaccine 56 days post-immunization. FIG. 6E shows the percent of SARS-CoV-2 WA1 Spike protein-specific plasma cells present in the bone marrow of mice immunized with Comirnaty (BNT162b2 mRNA, Pfizer-BioNTech), Comirnaty with alum, or the FDC vaccine 84 days post-immunization. FIG. 6F shows neutralization titers against SARS-CoV-2 WA1 after immunization with Comirnaty (BNT162b2 mRNA, Pfizer-BioNTech; “C”), Comirnaty with alum (“CA”), or the FDC vaccine (“CAM”). ns=not significant; * p<0.05; ** or §§ p<0.01; *** p<0.001; **** p<0.0001. Dotted line parallel to the x-axis indicates lower of detection.

FIGS. 7A-7K show alum-mannan expands germinal center responses. FIG. 7A shows a schematic of a vaccine administration schedule in which mice were administered saline, alum-mannan, Comirnaty (BNT162b2 mRNA, Pfizer-BioNTech), Comirnaty with alum, or Comirnaty with alum-mannan on day 0 and day 14. FIG. 7B shows CD4 T-cell counts in mouse draining lymph nodes collected 7 days post-prime with saline (“1”), alum-mannan (“2”), Comirnaty (“3”), or Comirnaty admixed with alum-mannan (“4”). FIG. 7C shows follicular T-cell counts in mouse draining lymph nodes collected 7 days post-prime with saline (“1”), alum-mannan (“2”), Comirnaty (“3”), or Comirnaty admixed with alum-mannan (“4”). FIG. 7D shows follicular T cell counts in mouse draining lymph nodes collected up to 84-days post-initial immunization with Comirnaty (BNT162b2 mRNA, Pfizer-BioNTech; “C”), Comirnaty with alum, (“CA”) or Comirnaty with alum-mannan (“CAM”). FIG. 7E shows fold-change in follicular T cell counts in mouse draining lymph nodes collected up to 84-days post-initial immunization with Comirnaty (BNT162b2 mRNA, Pfizer-BioNTech; “C”), Comirnaty with alum (“CA”), or Comirnaty with alum-mannan (“CAM”) relative to Comirnaty alone. FIG. 7F shows INF-γ levels in mouse draining lymph nodes collected up to 84-days post-initial immunization with Comirnaty (BNT162b2 mRNA, Pfizer-BioNTech; “C”), Comirnaty with alum (“CA”), or Comirnaty with alum-mannan (“CAM”). FIG. 7G shows B cell counts in mouse draining lymph nodes collected 7 days post-prime with saline (“1”), alum-mannan (“2”), Comirnaty (“3”), or Comirnaty admixed with alum-mannan (“4”). FIG. 7H shows germinal B cell counts in mouse draining lymph nodes collected 7 days post-prime with saline (“1”), alum-mannan (“2”), Comirnaty (“3”), or Comirnaty admixed with alum-mannan (“4”). FIG. 7I shows fold-change of germinal center B cells in mouse draining lymph nodes after vaccination with Comirnaty (“Control”), alum-mannan (“Alum+Mannan”) or Comirnaty admixed with alum-mannan (“Alum+Mannan+Pfizer”) relative to Comirnaty alone. FIG. 7J shows germinal center B cell counts in mouse draining lymph nodes collected up to 84-days post-initial immunization with Comirnaty (BNT162b2 mRNA, Pfizer-BioNTech), Comirnaty with alum, or Comirnaty with alum-mannan. FIG. 7K shows fold-change in germinal center B cell counts in mouse draining lymph nodes collected up to 84-days post-initial immunization with Comirnaty (BNT162b2 mRNA, Pfizer-BioNTech; “C”), Comirnaty with alum (“CA”), or Comirnaty with alum-mannan (“CAM”) relative to Comirnaty alone. ns=not significant; * p<0.05; ** p<0.01; *** p<0.001; **** p<0.0001.

FIGS. 8A-8H show alum-mannan allows the generation of B cells that recognize distinct variants of concern. FIG. 8A shows B cell counts in mouse draining lymph nodes that recognize the SARS-CoV-2 WA1, BA.4/BA.5/BA.5.2, or XBB.1.5 Spike protein after immunization with Comirnaty (BNT162b2 mRNA, Pfizer-BioNTech). FIG. 8B shows B cell counts in mouse draining lymph nodes that recognize the SARS-CoV-2 WA1 Spike protein after immunization with Comirnaty (“C”), Comirnaty and alum (“CA”), or Comirnaty and alum-mannan (“CAM”). Saline (“S”) and alum-mannan (“M”) were used as controls. FIG. 8C shows B cell counts in mouse draining lymph nodes that recognize the SARS-CoV-2 BA.4/BA.5/BA.5.2 Spike protein after immunization with Comirnaty (“C”), Comirnaty and alum (“CA”), or Comirnaty and alum-mannan (“CAM”). Saline (“S”) and alum-mannan (“M”) were used as controls. FIG. 8D shows B cell counts in mouse draining lymph nodes that recognize the SARS-CoV-2 XBB.1.5 Spike protein after immunization with Comirnaty (“C”), Comirnaty and alum (“CA”), or Comirnaty and alum-mannan (“CAM”). Saline (“S”) and alum-mannan (“M”) were used as controls. FIG. 8E shows fold change in B cell counts in mouse draining lymph nodes that recognize the SARS-CoV-2 WA1 Spike protein after immunization with Comirnaty (“C”), Comirnaty and alum (“CA”), or Comirnaty and alum-mannan (“CAM”), relative to B cell counts in mice receiving Comirnaty alone. FIG. 8F shows the gating strategy for IgG⁺CD38⁺ (top row) or IgG⁺CD80⁺PD-L2⁺ (bottom row) memory B cells. FIG. 8G shows IgG⁺CD38⁺ memory B cell counts 56 days post-administration of Comirnaty, Comirnaty and alum, or Comirnaty and alum-mannan that recognize SARS-CoV-2 WA1, BA.4/BA.5/BA.5.2, or XBB.1.5 Spike proteins. FIG. 8H shows IgG⁺CD80⁺PD-L2⁺ memory B cell counts 56 days post-administration of Comirnaty, Comirnaty and alum, or Comirnaty and alum-mannan that recognize SARS-CoV-2 WA1, BA.4/BA.5/BA.5.2, or XBB.1.5 Spike proteins. ns=not significant; * p<0.05; ** or §§ p<0.01; *** p<0.001; **** p<0.0001.

FIGS. 9A-9D show adjuvantation of Comirnaty with alum-mannan expands the formation of antibodies that neutralize distinct Omicron sublineages. Mice were immunized with Comirnaty (BNT162b2 mRNA, Pfizer-BioNTech; “C”), Comirnaty and alum (“CA”), or Comirnaty and alum-mannan (“CAM”) on day 0 and day 14. Neutralization titers of the SARS-CoV-2 pseudovirus expressing the Spike protein of SARS-CoV-2 BA.5 (FIG. 9A) or SARS-CoV-2 XBB.1.5 (FIG. 9B) were evaluated. Neutralization titers against the ancestral SARS-CoV-2 WA1, BA.1.1.529, and BA.5, expressed as the effective concentration 50 (EC₅₀) were measured at day 35 (FIG. 9C) or day 56 (FIG. 9D) after prime. ns=not significant; * p<0.05; ** or §§ p<0.01; *** p<0.001; **** p<0.0001. Dotted line parallel to the x-axis indicates lower of detection.

FIGS. 10A-10V shows glycan adjuvantation of Comirnaty alters the inflammatory programs in the draining lymph node early upon immunization. Mice were injected intramuscularly with saline, Comirnaty (BNT162b2 mRNA, Pfizer-BioNTech), or Comirnaty with alum, or Comirnaty with alum-mannan. Draining lymph nodes were collected 1-, 3-, or 7-days after immunization and bulk RNA sequencing was performed. FIG. 10A shows principal component analysis of indicated conditions. FIG. 10B shows heatmaps of Z-scored r log-normalized counts of top 50 genes by loadings (25 positive, 25 negative) of principal component (PC) 1 (left) and 2 (right) for all condition and samples. FIG. 10C shows bubble plots of MsigDB's Hallmark pathways pathway-enrichment analysis of Comirnaty-treated draining lymph nodes compared to saline-treated control lymph nodes 1-, 3-, and 7-days post injection. FIG. 10D shows a volcano plot of differentially expressed genes (DEGs) in the draining lymph nodes of mice treated with Comirnaty alone at day 7 compared to day 3 post-injection. FIG. 10E shows volcano plots of DEGs of mice treated with Comirnaty and alum, compared to Comirnaty alone, at day 1 (left), day 3 (middle) and day 7 (right) after injection. FIG. 10F shows volcano plot of DEGs (left) and a bubble plot of pathway-enrichment analysis (right) of mice treated with Comirnaty and alum-mannan, as compared to Comirnaty alone 1 day post-injection. FIG. 10G shows GSEA enrichment plots in mice immunized with Comirnaty and alum-mannan, compared to Comirnaty alone. Inflammasome pathways from the Reactome database (left) and Gene Ontology Biological process (right) are shown. FIG. 10H shows GSEA enrichment plots in mice immunized with Comirnaty and alum-mannan, compared to Comirnaty alone. FIG. 10I shows draining lymph node weights of mice treated with Comirnaty (“C”), Comirnaty and alum (“CA”), or Comirnaty and alum-mannan (“CAM”) relative to saline-treated controls. FIG. 10J shows absolute number of CD45⁺ cells per draining lymph node as assessed by flow cytometry in mice treated with Comirnaty (“C”), Comirnaty and alum (“CA”), or Comirnaty and alum-mannan (“CAM”). FIG. 10K shows GSEA enrichment plots of the C. albicans-response signature in mice immunized with Comirnaty and alum-mannan compared to Comirnaty alone. FIG. 10L shows a volcano plot (left) and bubble plot (right) of pathway enrichment analyses in mice treated with Comirnaty and alum-mannan, compared to Comirnaty alone, 3-days post-immunization. FIG. 10M shows GSEA enrichment plots of interferon alpha response (left) and interferon gamma response (right) signatures in mice treated with Comirnaty and alum-mannan, compared to Comirnaty alone, 3-days post-immunization. FIG. 10N shows a volcano plot (left) and bubble plot (right) of pathway enrichment analyses in mice treated with Comirnaty and alum-mannan, compared to Comirnaty alone, 7-days post-immunization. FIG. 10O shows GSEA enrichment plots of interferon alpha response (left) and interferon gamma response (right) signatures in mice treated with Comirnaty and alum-mannan, compared to Comirnaty alone, 7-days post-immunization. FIG. 10P shows a volcano plot (left) and bubble plot (right) of pathway enrichment analyses in mice treated with Comirnaty and alum-mannan, compared to Comirnaty and alum, 1-day post-immunization. FIG. 10Q shows GSEA enrichment plots of interferon alpha response (left), interferon gamma response (middle), and C. albicans-response signature (right) in mice treated with Comirnaty and alum-mannan, compared to Comirnaty and alum, 1-day post-immunization. FIG. 10R shows a volcano plot (left) and bubble plot (right) of pathway enrichment analyses in mice treated with Comirnaty and alum-mannan, compared to Comirnaty and alum, 3-day post-immunization. FIG. 10S shows GSEA enrichment plots of interferon alpha response (left), interferon gamma response (middle), and C. albicans-response signature (right) in mice treated with Comirnaty and alum-mannan, compared to Comirnaty and alum, 3-day post-immunization. FIG. 10T shows a volcano plot (left) and bubble plot (right) of pathway enrichment analyses in mice treated with Comirnaty and alum-mannan, compared to Comirnaty and alum, 7-day post-immunization. FIG. 10U shows GSEA enrichment plots of interferon alpha response (left), interferon gamma response (middle), and C. albicans-response signature (right) in mice treated with Comirnaty and alum-mannan, compared to Comirnaty and alum, 7-day post-immunization. FIG. 10V shows GSEA enrichment plots of the C. albicans-response signature for mice immunized with Comirnaty and alum-mannan, compared to Comirnaty alone. Only pathways with an adjusted p-value<0.05 are shown. ns=not significant; * p<0.05; ** or §§ p<0.01; *** or §§§ p<0.001; **** or §§§§ p<0.0001. Dotted lines indicate an adjusted p-value of 0.01 and a log 2 fold change of 1.

FIGS. 11A-11K show alum-mannan controls the inflammatory programs of human phagocytes. Human PBMCs without GM-CSF pre-treatment were stimulated with saline, alum (4 μg/ml), mannan (10 μg/ml), alum-mannan (4 μg/ml alum co-formulated with 10 μg/ml mannan), β-glucan (10 μg/ml), or LPS (1 μg/ml). 24 hours after stimulation, cell culture supernatant was collected and IL-1β (FIG. 11A), IL-6 (FIG. 11B), and TNF (FIG. 11C) were measured by ELISA. FIG. 11D shows IL-1β (left), IL-6 (middle), and TNF (right) levels in human PBMCs cultured for 16 hours with GM-CSF and stimulated with saline, alum (4 μg/ml), mannan (10 μg/ml), alum-mannan (4 μg/ml alum co-formulated with 10 μg/ml mannan), β-glucan (10 μg/ml), or LPS (1 μg/ml). FIG. 11E shows IL-1β (left), IL-6 (middle), and TNF (right) levels in human monocyte-derived dendritic cells stimulated with saline, alum (4 μg/ml), mannan (10 μg/ml), alum-mannan (4 μg/ml alum co-formulated with 10 μg/ml mannan), β-glucan (10 μg/ml), or LPS (1 μg/ml). Human GM-CSF PBMCs were stimulated with saline, β-glucan (10 μg/ml), or LPS (1 μg/ml) or increasing doses of alum (2-10 μg/ml) with or without mannan (10 μg/ml or 20 μg/ml). 24 hours after stimulation, cell culture supernatants were collected and IL-1β (FIG. 11F), IL-6 (FIG. 11G), TNF (FIG. 11H), and pro-IL-1β (FIG. 11I) were measured by ELISA. FIG. 11J shows Western blot analysis of human GM-CSF PBMCs simulated as in FIG. 11D. FIG. 11K shows a schematic of the activity of alum-mannan: co-administration of alum-mannan with Comirnaty prolong and enhances interferon (IFN) responses and inflammasome activation, promoting germinal center expansion. This amplifies antibody (Ab) diversity, enhancing protection against SARS-CoV-2 variants of concern. ns=not significant; * p<0.05; ** p<0.01; *** p<0.001; **** p<0.0001.

FIGS. 12A-12H show the immunomodulatory activity of Konjac glucomannan. FIG. 12A shows lymph node weight of mice injected intradermally with 500 μg C. albicans-derived mannan (Mannan) or 500 μg synthetic polymannose (PolyMannose) compared to contralaterally saline-injected lymph nodes (Control). FIG. 12B shows fold-change in interferon induced protein with tetratricopeptide repeats 2 (IFIT2) mRNA expression after intradermal administration of C. albicans-derived mannan (Mannan) or synthetic polymannose (Polymannose) relative to IFIT2 mRNA expression in contralateral saline-injected lymph node. FIG. 12C shows fold-change in guanylate binding protein 2 (GBP2) mRNA expression after intradermal administration of C. albicans-derived mannan (Mannan) or synthetic polymannose (Polymannose) relative to GBP2 mRNA expression in contralateral saline-injected lymph node. FIG. 12D shows fold-change in lymph node weights of mice injected intradermally with 10 μg, 50 μg, 100 μg, or 500 μg Konjac glucomannan relative to contralateral saline-injected lymph node 24 hours post-administration. FIG. 12E shows CD45+ T cell numbers in the lymph node of mice 24 hours post-administration of saline or 500 μg Konjac glucomannan. FIG. 12F shows fold-change in radical S-adenosyl methionine domain-containing protein 2 (RSAD2) mRNA expression after intradermal administration of Konjac glucomannan relative to RSAD2 mRNA expression in contralateral saline-injected lymph node. FIG. 12G shows fold-change in GBP2 mRNA expression after intradermal administration of Konjac glucomannan relative to mRNA expression of GBP2 in contralateral saline-injected lymph node. FIG. 12H shows fold-change in IFIT2 mRNA expression after intradermal administration of Konjac glucomannan relative to mRNA expression of IFIT2 in contralateral saline-injected lymph node. ns=not significant; * p<0.05; ** p<0.01; *** p<0.001; p<0.0001.

FIGS. 13A-13F show Konjac glucomannan drives Dectin-2 dependent responses and can be formulated in a FDC vaccine that incorporates Konjac glucomannan, alum, and Comirnaty (FDCakgm vaccine). FIG. 13A shows lymph node weights of wild-type (WT) mice or Dectin-2-deficient (Dectin-2 KO) mice 24 hours post-intradermal administration of Konjac glucomannan (10 μg/mouse) as compared to weight of contralateral saline-injected lymph node. FIG. 13B shows lymph node CD45+ T cell numbers in wild-type (WT) mice or Dectin-2-deficient (Dectin-2-KO) mice 24 hours post-intradermal administration of Konjac glucomannan (10 μg/mouse) as compared to CD45+ T cell numbers in contralateral saline-injected lymph node. FIG. 13C shows lymph node weights of mice 1, 7, and 14 days post-intradermal administration of saline (Control), 500 μg C. albicans-derived mannan (Mannan), 500 μg C. albicans-derived mannan with 200 μg alum (Alum+Mannan), or 100 μg Konjac glucomannan (Glucomannan) relative to the weight of contralateral saline-injected lymph nodes. FIG. 13D shows lymph node weights of mice 24 hours post-intradermal administration of 60 μg Konjac glucomannan or Konjac glucomannan formulated with alum (1:3 alum:mannan weight ratio) compared to the weight of contralateral saline-injected lymph node. FIG. 13E shows released mRNA of Comirnaty (BNT162b2 mRNA, Pfizer-BioNTech; “Pfizer”) alone or incubated with alum-mannan in a 1:5 alum:mannan weight ratio (AM5) at a 1:1, 1:10, or 1:100 ratio of Comirnaty:alum-mannan, alum-glucomannan in a 1:5 alum:glucomannan weight ratio (AGM5) at a 1:1, 1:10, or 1:100 ratio of Comirnaty:alum-glucomannan, or alum-glucomannan in a 1:3 alum:glucomannan weight ratio (AGM3) at a 1:1, 1:10, or 1:100 ratio of Comirnaty:alum-glucomannan measured after 24 hours at 4° C. FIG. 13F shows that the mRNA vaccine Comirnaty remained relatively stable when formulated with or without alum-mannan or alum-glucomannan, releasing <10% of mRNA into solution across all constructs tested. ns=not significant; * p<0.05; ** p<0.01; *** p<0.001; **** p<0.0001.

FIGS. 14A-14B show FDC and FDCakgm vaccines provide superior protection against ancestral SARS-CoV-2 and the Omicron (B.1.1.529) variant of concern. FIG. 14A shows IgG levels in mice immunized with Comirnaty (BNT162b2 mRNA, Pfizer-BioNTech; “Control”), Comirnaty with alum-mannan (“Pfizer+AM”), Comirnaty with glucomannan (“Pfizer+GM”), or Comirnaty with alum-glucomannan (“Pfizer+AGM”) before priming (day 0), 14 days post-priming (day 14), 14 days post-boosting (day 28), or 23 days post-boosting (day 37). FIG. 14B shows the titer of neutralizing antibody against SARS-CoV-2 WA1 and SARS-CoV-2 B.1.1.529 in the sera of mice immunized with Comirnaty (BNT162b2 mRNA, Pfizer-BioNTech; “Pfizer”), Comirnaty with alum-mannan (“Pfizer+AM”), Comirnaty with glucomannan (“Pfizer+GM”), or Comirnaty with alum-glucomannan (“Pfizer+AGM”) before priming (day 0), 14 days post-priming (day 14), 14 days post-boosting (day 28), or 23 days post-boosting (day 37). ns=not significant; * p<0.05; ** p<0.01; *** p<0.001; p<0.0001.

FIGS. 15A-15C show a comparison of Comirnaty adjuvantation with alum-mannan or AS04. FIG. 15A shows anti-Spike IgG levels in mice immunized with Comirnaty (BNT162b2 mRNA, Pfizer-BioNTech; “C”) alone, Comirnaty with AS04 (“C-AS04”), or Comirnaty with alum-mannan (“C-MA”). FIG. 15B shows neutralization titers against the Spike protein of SARS-CoV-2 WA1 with D614G mutation, SARS-CoV-2 BA.1, SARS-CoV-2 BA.5, and SARS-CoV-2 XBB.1.5 56 days post-initial immunization with Comirnaty (BNT162b2 mRNA, Pfizer-BioNTech; “C”) alone, Comirnaty with AS04 (“C-AS04”), or Comirnaty with alum-mannan (“C-MA”). FIG. 15C shows draining lymph node follicular T cell counts, germinal center B cell counts, counts of germinal center B cells expressing a B cell receptor specific for the SARS-CoV-2 WA1 Spike protein, counts of germinal center B cells expressing a B cell receptor specific for the SARS-CoV-2 BA.4/BA.5 Spike protein, and counts of germinal center B cells expressing a B cell receptor specific for SARS-CoV-2 XBB.1.5 Spike protein 56 days post-initial immunization with Comirnaty (BNT162b2 mRNA, Pfizer-BioNTech; “C”) alone, Comirnaty with AS04 (“C-AS04”), or Comirnaty with alum-mannan (“C-MA”). ns=not significant; * p<0.05; ** p<0.01; *** p<0.001; **** p<0.0001.

FIG. 16 shows anti-Spike IgG levels up to 300 days post-immunization with Comirnaty (BNT162b2 mRNA, Pfizer-BioNTech; “C”), Comirnaty and alum (“C-A”), or Comirnaty and alum-mannan (“C-MA”). ns=not significant; * p<0.05; ** p<0.01; *** p<0.001; **** p<0.0001.

FIGS. 17A-17B show germinal center B cell counts expressing B cell receptors specific to the Spike protein of variants of concern. FIG. 17A shows germinal center B cell counts expressing B cell receptors specific to SARS-CoV-2 BA.4/BA.5 Spike protein 14, 21, 28, 35, 56, and 84 days post-initial immunization with Comirnaty (BNT162b2, Pfizer-BioNTech; “C”), Comirnaty and alum (“C-A”), or Comirnaty and alum-mannan (“C-MA”). FIG. 17B shows germinal center B cell counts expressing B cell receptors specific to SARS-CoV-2 XBB.1.5 Spike protein 14, 21, 28, 35, 56, and 84 days post-initial immunization with Comirnaty (BNT162b2, Pfizer-BioNTech; “C”), Comirnaty and alum (“C-A”), or Comirnaty and alum-mannan (“C-MA”).

### DETAILED DESCRIPTION

Adjuvants are important components of vaccines, enhancing the strength, breadth, and persistence of immune responses against pathogens. However, few vaccine adjuvants are included in licensed vaccines due to safety or tolerability concerns. Furthermore, despite their widespread use, the molecular mechanisms by which available adjuvants work are not well understood.

Some aspects of the present disclosure provide an immunogenic composition comprising an adjuvantation system comprising a mannan and an immunogen. In some embodiments, the immunogen is a nucleic acid or a protein. Alternatively, in some aspects of the present disclosure provide immunogenic compositions (e.g., vaccine compositions) comprising an adjuvantation system and a nucleic acid encoding an antigen. In some embodiments, the adjuvantation system comprises a mannan. In some embodiments, the adjuvantation system comprises a plant mannan and an aluminum salt. In some embodiments, the adjuvantation system comprises a fungal mannan and an aluminum salt. Other aspects of the present disclosure provide immunogenic composition (e.g., vaccine composition) comprising an adjuvantation system and an antigen, wherein the adjuvantation system comprises a plant mannan.

An “adjuvantation system” refers to a composition comprising one or more adjuvants. An “adjuvant” refers to a pharmacological or immunological agent that modifies the effect of other agents, for example, of a vaccine or vaccine components (e.g., antigen, mRNA, lipids). Adjuvants are typically included in vaccines to enhance the recipient subject's immune response to an antigen. The use of adjuvants allows the induction of a greater immune response in a subject with the same dose of antigen, the induction of a similar level of immune response with a lower dose of antigen, and/or the induction of a prolonged immune response in a subject with the same dose of antigen. Adjuvants may also allow the induction of cross-reactive antibody responses against multiple variants, thus broadening the immune response. Adjuvants are thought to function in several ways, including by increasing the surface area of antigen, prolonging the retention of the antigen in the body thus allowing time for the lymphoid system to have access to the antigen, slowing the release of antigen, targeting antigen to macrophages, activating macrophages, activating leukocytes such as antigen-presenting cells (e.g., monocytes, macrophages, and/or dendritic cells), or otherwise eliciting broad activation of the cells of the immune system. The ability of an adjuvant to induce and increase a specific type of immune response and the identification of that ability is thus a key factor in the selection of particular adjuvants for vaccine use against a particular pathogen. Adjuvants that are known to those of skill in the art, include, without limitation: aluminum salts (e.g., aluminum hydroxide, aluminum phosphate, aluminum hydroxyphosphate, aluminum potassium sulfate; collectively referred to herein as “alum”), liposomes, lipopolysaccharide (LPS) or derivatives thereof such as monophosphoryl lipid A (MPLA) and glycopyranosyl lipid A (GLA), molecular cages for antigen, endocytosed nucleic acids such as double-stranded RNA (dsRNA), single-stranded DNA (ssDNA), and unmethylated CpG dinucleotide-containing DNA. Typical adjuvants include water and oil emulsions, e.g., Freund's adjuvant and MF59, and chemical compounds such as alum. At present, currently licensed vaccines in the United States contain only a limited number of adjuvants, such as alum which enhances production of T helper type 2 (Th2) cells, and MPLA which activates innate immunity via Toll-like receptor 4 (TLR4).

In some embodiments, an adjuvantation system of the present disclosure comprises a mannan. Mannans are polysaccharides of D-mannose joined by 3-(1,4) linkages that can be found in various natural sources, such as plants and fungi. A mannan may be a branched polysaccharide or a linear polysaccharide. A mannan may be a derivative mannan, such as, for instance, a shortened (i.e., lower molecular weight) or elongated (i.e., higher molecular weight) version of a mannan (e.g., a plant mannan or a fungal mannan). A mannan may be in native, oxidated, or reduced form of a mannan (e.g., a plant mannan or a fungal mannan). A mannan may be covalently conjugated to another chemical moiety, such as but not limited to a protein (e.g., a glycoprotein). In some embodiments, a mannan may be composed of one or more than one type of carbohydrate monomer covalently linked in such a way as to form a polysaccharide. In some embodiments, at least 30% (e.g., at least 30%, 40%, 50%, 60%, 70%, 80%, 90%, or 99%) of the mannose saccharides are connected by 3-(1,4) linkages. In some embodiments, a mannan comprises β-1,4-linked backbone. In some embodiments, the β-1,4-linked backbone is substituted with side chains of α-1,6-linked galactose residues. A mannan (e.g., a plant mannan or a fungal mannan) may be soluble, partially soluble, or insoluble in solution, particularly in an aqueous solution. A mannan (e.g., a plant mannan or a fungal mannan) may have antigenic properties (i.e., activates an immune response in an animal or human subject). In some embodiments, the mannan is a glucomannan, a galactomannan, or a galactoglucomannan. In some embodiments, the mannan is a plant mannan or a fungal mannan.

In some embodiments, the mannan is a fungal mannan. A fungal mannan may be produced and secreted by a fungal cell or occur as a component of a fungal cell (e.g., as a structural component of a fungal cell wall). In some embodiments, the fungal mannan elicits an immune response in a subject. In some embodiments, the fungal mannan is Candida mannan. In some embodiments, the fungal mannan is from a pathogenic fungus that elicits an immune response, such as Candida albicans (C. albicans). In some embodiments, the fungal mannan is from C. albicans. In some embodiments, the fungal mannan is a C. albicans mannan.

In some embodiments, the mannan is a plant mannan. A plant mannan may be produced and secreted by a plant cell or occur as a component of a plant cell (e.g., as a structural component of a plant cell). For example, in some embodiments, the plant mannan is derived from Ceratonia siliqua, Phytelephas macrocarpa, Schizolobium amazonicum, Schizolobium parahybum, Carum carvi, Cyamopsis tetragonolobus, Amorphophallus konjac, Coffea arabica, Aloe barbadensis, and/or Cesalpinia spinosa. In some embodiments, the plant mannan is a glucomannan. A glucomannan is a water-soluble polysaccharide comprising β-(1,4)-linked D-mannose and D-glucose. The ratio of D-mannose to D-glucose in a glucomannan is typically about 1.6:1. In some embodiments, the plant mannan is from konjac, salep, or conifers (e.g., cedars, Douglas firs, cypresses, firs, junipers, kauri, larches, pines, hemlocks, redwoods, spruces, yews). In some embodiments, the plant mannan is a konjac mannan, a salep mannan, or a conifer mannan (e.g., a cedar mannan, a Douglas fir mannan, a cypress mannan, a fir mannan, a juniper mannan, a kauri mannan, a larch mannan, a pine mannan, a hemlock mannan, a redwood mannan, a spruce mannan, a yew mannan). In some embodiments, the plant mannan is a glucomannan from a konjac plant. In some embodiments, the plant mannan is a konjac glucomannan. In some embodiments, the plant mannan is a galactomannan. A galactomannan is a polysaccharide comprising β-(1,4)-linked D-mannose with (1,6)-linked D-galactose. The ratio of D-mannose to D-galactose may vary from about 1:1 to about 1:5. In some embodiments, the plant mannan is from fenugreek gum, guar gum, tara gum, locust bean gum, or cassia gum. In some embodiments, the plant mannan is a fenugreek gum mannan, a guar gum mannan, a tara gum mannan, a locust bean gum mannan, or a cassia gum mannan. In some embodiments, the plant mannan is a galactoglucomannan. A galactoglucomannan is a water-soluble polysaccharide comprising β-(1,4,)-linked D-mannose and D-glucose units with (1,6)-linked D-galactose units attached to D-mannose units. In some embodiments, the plant mannan is from a Norway spruce. In some embodiments, the plant mannan is a Norway spruce mannan.

In some embodiments, the adjuvantation system further comprises an aluminum salt. In some embodiments, the aluminum salt is aluminum hydroxide, aluminum phosphate, or aluminum hydroxyphosphate. In some embodiments, the aluminum salt is Alhydrogel® (InvivoGen, USA). In some embodiments, the adjuvantation system comprises a mannan (e.g., a plant mannan or a fungal mannan) and an aluminum salt. In some embodiments, the adjuvantation system comprises a plant mannan (e.g., a konjac glucomannan) and an aluminum salt. In some embodiments, the adjuvantation system comprises a konjac glucomannan and an aluminum salt. In some embodiments, the adjuvantation system comprises a fungal mannan (e.g., a C. albicans mannan) and an aluminum salt. In some embodiments, the adjuvantation system comprises a C. albicans mannan and an aluminum salt.

In some embodiments, the mannan (e.g., a fungal mannan or a plant mannan) is admixed with the aluminum salt. In some embodiments, in an adjuvantation system comprising a fungal mannan (e.g., a C. albicans mannan) and an aluminum salt, the fungal mannan is admixed with the aluminum salt. In some embodiments, in an adjuvantation system comprising a plant mannan (e.g., a konjac glucomannan) and an aluminum salt, the plant mannan is admixed with the aluminum salt. Where a mannan (e.g., a plant mannan or a fungal mannan) is admixed with an aluminum salt, the quantity of each component in the adjuvantation system may be varied according to the desired properties or effects in the final admixture. In some embodiments, the adjuvantation system comprises equal quantities of aluminum salt and mannan (e.g., having a 1:1 weight ratio of aluminum salt to mannan). In some embodiments, the adjuvantation comprises different quantities of aluminum salt and mannan. In some embodiments, the weight ratio of aluminum salt to mannan in the adjuvantation system is between 1:1 and 1:100. A “weight ratio” is a comparison of the weights of different substances in a mixture or a compound, expressed as the proportion of one substance to another based on their respective weights. For example, an adjuvantation system comprising a 1:1 weight ratio of aluminum salt to mannan may comprise 100 μg of aluminum salt and 100 μg of mannan, while an adjuvantation system comprising a 1:100 weight ratio of aluminum salt to mannan may comprise 1 μg of aluminum salt and 100 μg of mannan. In some embodiments, the weight ratio of aluminum salt to mannan in the adjuvantation system is 1:1. In some embodiments, the weight ratio of aluminum salt to mannan in the adjuvantation system is 1:2. In some embodiments, the weight ratio of aluminum salt to mannan in the adjuvantation is 1:3. In some embodiments, the weight ratio of aluminum salt to mannan in the adjuvantation system is 1:4. In some embodiments, the weight ratio of aluminum salt to plant mannan (e.g., konjac glucomannan) in the adjuvantation system is 1:4. In some embodiments, the weight ratio of aluminum salt to konjac glucomannan in the adjuvantation system is 1:4. In some embodiments, the weight ratio of aluminum salt to mannan in the adjuvantation system is 1:5. In some embodiments, the weight ratio of aluminum salt to fungal mannan (e.g., C. albicans mannan) in the adjuvantation system is 1:5. In some embodiments, the weight ratio of an aluminum salt to C. albicans mannan in the adjuvantation system is 1:5. In some embodiments, the weight ratio of aluminum salt to mannan in the adjuvantation system is 1:10. In some embodiments, the weight ratio of aluminum salt to mannan in the adjuvantation system is 1:100.

An “immunogenic composition” refers to a composition comprising (i) an adjuvant (e.g., a mannan) and (ii) a nucleic acid encoding an antigen and/or an antigen. In some embodiments, an immunogenic composition of the present disclosure comprises an antigen. An “antigen” refers to an entity that is bound by an antibody or receptor, or an entity that induces the production of the antibody. In some embodiments, an antigen increases the production of antibodies that specifically bind the antigen. In some embodiments, an antigen comprises a protein or polypeptide. Such a protein or polypeptide is referred to herein as an “immunogenic polypeptide.” In some embodiments, the antigen is from a microbial pathogen. In some embodiments, the antigen is a bacterial antigen, a fungal antigen, or a viral antigen. In some embodiments, the antigen may comprise parts (e.g., coats, capsules, cell walls, flagella, fimbriae, toxins) of bacteria, viruses, fungi, and other microorganisms.

In some embodiments, the antigen is a bacterial antigen. A “bacterial antigen” is an antigen that originates from bacteria or, in the case of protein and polypeptide antigens, has a sequence that is identical or substantially similar (homologous) to an endogenous bacterial protein or bacterial polypeptide. In some embodiments, the bacterial antigen may comprise parts of Campylobacter spp., Clostridioides difficile, Haemophilus ducreyi, Clostridium perfringens, Escherichia coli, Klebsiella granulomatis, Haemophilus influenzae, Legionella spp., Leptospira spp., Listeria monocytogenes, Borrelia burgdorferi, Borrelia mayonii, Neisseria meningitidis, Yersinia pestis, Chlamydia psittaci, Coxiella burnetii, Ricinus communis, Rickettsia rickettsia, Salmonella spp., Shigella spp., Staphylococcus aureus, Streptococcus spp., Clostridium tetani, Mycobacterium tuberculosis, Francisella tularensis, Salmonella typhi, Salmonella paratyphi, Vibrio cholerae, or Yersinia enterocolitica.

In some embodiments, the antigen is a fungal antigen. A “fungal antigen” is an antigen that originates from fungi or, in the case of protein and polypeptide antigens, has a sequence that is identical or substantially similar (homologous) to an endogenous fungal protein or fungal polypeptide. In some embodiments, the fungal antigen may comprise parts of Coccidioides spp., Blastomyces spp., Cryptococcus gattii, Histoplasma spp., Paracoccidioides spp., Pneumocystis jirovecii, Candida spp., Cryptococcus neoformans, Talaromyces spp., or Aspergillus spp.

In some embodiments, the antigen is a viral antigen. A “viral antigen” is an antigen that originates from a virus or, in the case of protein and polypeptide antigens, has a sequence that is identical or substantially similar (homologous) to an endogenous viral protein or viral polypeptide. In some embodiments, the viral antigen may comprise parts of an Alpha coronavirus, a Beta coronavirus (e.g., MERS-CoV, SARS-CoV-1, SARS-CoV-2), Alphavirus (e.g., Barmah Forest virus, Chikungunya virus, Eastern equine encephalitis virus, Getah virus, Sagiyama virus, Mayaro virus, O'nyong-nyong virus, Ross river virus, Semliki forest virus, Sindbis virus, Venezuelan equine encephalitis virus, Western equine encephalitis virus), Arenavirus (e.g., Junin arenavirus, Lassa virus, Lymphocytic choriomeningitis virus, Machupo virus), Bandavirus (e.g., Severe fever with thrombocytopenia syndrome virus), Bocaparvovirus (e.g., Human bocavirus), Cardiovirus (e.g., Encephalomyocarditis virus), Coltivirus (e.g., Colorado tick fever virus, California hare coltivirus, Eyach virus), Cytomegalovirus (e.g., Human cytomegalovirus), Deltaretrovirus (e.g., Human T-lymphotropic virus), Delta virus (e.g., Hepatitis delta virus), Ebolavirus, Enterovirus (e.g., Human rhinovirus A, Human rhinovirus B, Human rhinovirus C, Coxsackievirus A, Coxsackievirus B, Echovirus, Human enterovirus D68, Poliovirus), Erythrovirus (e.g., Human parvovirus B19), Flavivirus (e.g., Murray valley encephalitis virus, Dengue virus, Saint Louis encephalitis virus, Tick-borne encephalitis virus, Powassan virus, Kyasanur Forest disease virus, Alkhurma virus, Omsk hemorrhagic fever virus, West Nile virus, Yellow fever virus, Zika virus), Hantavirus (e.g., Puumala virus, Saaremaa virus, Dobrava virus), Henipavirus (e.g., Hendra virus, Langya virus, Nipah virus), Hepacivirus (e.g., Hepatitis C virus), Hepatovirus (e.g., Hepatitis A virus), Hepevirus (e.g., Hepatitis E virus), Influenzavirus A, Influenzavirus B, Influenzavirus C, Lentivirus (e.g., Human immunodeficiency virus 1, Human immunodeficiency virus 2), Lymphocryptovirus (e.g., Epstein-Barr virus), Lyssavirus (e.g., Mokola virus, Lagos bat virus, Shimoni bat virus, Rabies virus, Aravan virus, Duvenhage virus, Australian bat lyssavirus, European bat lyssavirus, Irkut virus, Khujand virus), Mamastrovirus (e.g., Human astrovirus), Marburgvirus (e.g., Lake Victoria Marburgvirus), Mastadenovirus (e.g., Human adenovirus), Metapneumovirus (e.g., Human metapneumovirus), Molluscipoxvirus (e.g., Molluscum contagiosum virus), Morbilivirus (e.g., Measles virus), Norovirus, Orthobunyavirus (e.g., Oropouche virus, Batai virus, Bunyamwera virus, Cache Valley virus, Ngari virus), Orthoflavivirus (e.g., Japanese encephalitis virus), Orthohantavirus (e.g., Hantaan virus, Sin Nombre virus), Orthohepadnavirus (e.g., Hepatitis B virus), Orthonairovirus (e.g., Dugbe virus, Crimean-Congo hemorrhagic fever virus), Orthopneumovirus (e.g., Respiratory syncytial virus), Orthopoxvirus (e.g., Monkeypox virus, Cowpox virus, Small pox virus, Tanapox virus, Yaba-like disease virus, Vaccinia virus, Yaba monkey tumor virus), Orthorubulavirus (e.g., Human parainfluenzavirus 2, Human parainfluenzavirus 4), Papillomaviridae (e.g., Human papillomavirus), Parapoxvirus (e.g., Orf virus), Phlebovirus (e.g., Punta toro phlebovirus, Rift valley fever virus, Sandfly fever Naples phlebovirus, Uukuniemi virus), Polyomavirus (e.g., BK polyomavirus, JC polyomavirus, KI polyomavirus, Merkel cell polyomavirus, WU polyomavirus), Respirovirus (e.g., Human parainfluenzavirus 1, Human parainfluenzavirus 3), Rhadinovirus (e.g., Human herpesvirus 8), Rosavirus (e.g., Rosavirus A), Rotavirus (e.g., Rotavirus A, Rotavirus B, Rotavirus C), Rubivirus (e.g., Rubella virus), Rubulavirus (e.g., Mumms virus), Salivirus (e.g., Salivirus A), Sapovirus (e.g., Sapporo virus), Seadornavirus (e.g., Banna virus), Simplexvirus (e.g., Human herpesvirus 1, Human herpesvirus 2, Macacine alphaherpesvirus), Thogotovirus (e.g., Dhori virus, Batken virus, Bourbon virus, Thogotobirus), Torovirus (e.g., Human torovirus), or Varicellovirus (e.g., Varicella-zoster virus), Vesiculovirus (e.g., Chandipura virus, Indiana vesiculovirus, Isfahan virus). In some embodiments, the antigen comprises a nucleic acid derived from or encode components of Orthomyxoviruses, Hepatitis C Virus (HCV), Ebola disease, polio, measles, adult Human T-cell lymphotropic virus type 1 (HTLV-1), lymphocytic choriomeningitis virus, human immunodeficiency virus (HIV), rhinoviruses, Influenza, SARS, MERS, COVID-19, Dengue virus, Hepatitis C, Hepatitis E, West Nile fever, Ebola virus, respiratory syncytial virus, Rabies, mumps virus, hantavirus, Marburg virus, Lassa virus, parainfluenza, Monkey pox or combinations thereof.

In some embodiments, the composition comprises a combination of two or more nucleic acids. In some embodiments, the combination of two or more nucleic acids are derived from or encode components of influenza virus and coronavirus. In some embodiments, the combination of two or more nucleic acids are derived from influenza virus, coronavirus and respiratory syncytial virus.

In some embodiments, the immunogenic compositions described herein comprise an antigen, wherein the antigen comprises a Beta coronavirus protein or polypeptide. “Beta coronavirus” is one of four genera (Alpha-, Beta-, Gamma-, and Delta-) of coronaviruses. Beta coronaviruses belong to the subfamily Orthocoronavirinae in the family Coronaviridae, of the order Nidovirales. They are enveloped, positive-sense, single-stranded RNA viruses of zoonotic origin. Beta coronaviruses of the greatest clinical significance to humans include SARS-CoV-1 (which causes Severe Acute Respiratory Syndrome, SARS), SARS-CoV-2 (which causes the disease Coronavirus Disease 2019, COVID-19), and MERS-CoV (which causes Middle East Respiratory Syndrome, MERS). In some embodiments, the Beta coronavirus protein or polypeptide comprises a Beta coronavirus spike protein, a Beta coronavirus nucleocapsid protein, a Beta coronavirus membrane protein, a Beta coronavirus envelope protein, or an immunogenic fragment thereof (e.g., a Beta coronavirus spike protein receptor binding domain (RBD)). In some embodiments, the antigen comprises a MERS-CoV spike protein, a MERS-CoV nucleocapsid protein, a MERS-CoV membrane protein, a MERS-CoV envelope protein, or an immunogenic fragment thereof (e.g., a MERS-CoV spike protein RBD). In some embodiments, the antigen comprises a SARS-CoV-1 spike protein, a SARS-CoV-1 nucleocapsid protein, a SARS-CoV-1 membrane protein, a SARS-CoV-1 envelope protein, or an immunogenic fragment thereof (e.g., a SARS-CoV-1 spike protein RBD). In some embodiments, the antigen comprises a SARS-CoV-2 spike protein, a SARS-CoV-2 nucleocapsid protein, a SARS-CoV-2 membrane protein, a SARS-CoV-2 envelope protein, or an immunogenic fragment thereof (e.g., a SARS-CoV-2 spike protein RBD). In some embodiments, the viral antigen comprises an antigen from human papillomavirus. In some embodiments, the antigen from human papillomavirus is selected from L1 antigen, E6 antigen, E7 antigen, or a combination thereof. In some embodiments, the viral antigen comprises an antigen from hepatitis B virus. In some embodiments, the antigen from hepatitis B virus is HBsAg. In some embodiments, the viral antigen comprises an antigen from Epstein-Barr virus. In some embodiments, the antigen from Epstein-Barr virus is Epstein-Barr nuclear antigen. In some embodiments, the viral antigen is SV40.

Amino acid sequences of example Beta coronavirus antigens comprised by the immunogenic compositions described herein are provided in Table 1.

TABLE 1
Beta coronavirus protein antigens.

| Antigen | Amino Acid Sequence |
|---|---|
| SARS-CoV-1 | MFIFLLFLTLTSGSDLDRCTTFDDVQAPNYTQHTSSMRGVYYPDEIFRSDTLYLTQDLFLPFYSNVT |
| Spike Protein | GFHTINHTFGNPVIPFKDGIYFAATEKSNVVRGWVFGSTMNNKSQSVIIINNSINVVIRACNFELCD |
| (SEQ ID NO: 1) | NPFFAVSKPMGTQTHTMIFDNAFNCTFEYISDAFSLDVSEKSGNFKHLREFVFKNKDGFLYVYKGYQ |
| | PIDVVRDLPSGFNTLKPIFKLPLGINITNFRAILTAFSPAQDIWGTSAAAYFVGYLKPTTFMLKYDE |
| | NGTITDAVDCSQNPLAELKCSVKSFEIDKGIYQTSNFRVVPSGDVVRFPNITNLCPFGEVFNATKFP |
| | SVYAWERKKISNCVADYSVLYNSTFFSTFKCYGVSATKLNDLCFSNVYADSFVVKGDDVRQIAPGQT |
| | GVIADYNYKLPDDFMGCVLAWNTRNIDATSTGNYNYKYRYLRHGKLRPFERDISNVPFSPDGKPCTP |
| | PALNCYWPLNDYGFYTTTGIGYQPYRVVVLSFELLNAPATVCGPKLSTDLIKNQCVNFNFNGLTGTG |
| | VLTPSSKRFQPFQQFGRDVSDFTDSVRDPKTSEILDISPCSFGGVSVITPGTNASSEVAVLYQDVNC |
| | TDVSTAIHADQLTPAWRIYSTGNNVFQTQAGCLIGAEHVDTSYECDIPIGAGICASYHTVSLLRSTS |
| | QKSIVAYTMSLGADSSIAYSNNTIAIPTNFSISITTEVMPVSMAKTSVDCNMYICGDSTECANLLLQ |
| | YGSFCTQLNRALSGIAAEQDRNTREVFAQVKQMYKTPTLKYFGGFNFSQILPDPLKPTKRSFIEDLL |
| | FNKVTLADAGFMKQYGECLGDINARDLICAQKFNGLTVLPPLLTDDMIAAYTAALVSGTATAGWTFG |
| | AGAALQIPFAMQMAYRFNGIGVTQNVLYENQKQIANQFNKAISQIQESLTTTSTALGKLQDVVNQNA |
| | QALNTLVKQLSSNFGAISSVLNDILSRLDKVEAEVQIDRLITGRLQSLQTYVTQQLIRAAEIRASAN |
| | LAATKMSECVLGQSKRVDFCGKGYHLMSFPQAAPHGVVFLHVTYVPSQERNFTTAPAICHEGKAYFP |
| | REGVFVFNGTSWFITQRNFFSPQIITTDNTFVSGNCDVVIGIINNTVYDPLQPELDSFKEELDKYFK |
| | NHTSPDVDLGDISGINASVVNIQKEIDRLNEVAKNLNESLIDLQELGKYEQYIKWPWYVWLGFIAGL |
| | IAIVMVTILLCCMTSCCSCLKGACSCGSCCKFDEDDSEPVLKGVKLHYT |
| SARS-CoV-1 | RVVPSGDVVRFPNITNLCPFGEVFNATKFPSVYAWERKKISNCVADYSVLYNSTFFSTFKCYGVSAT |
| Spike Protein | KLNDLCFSNVYADSFVVKGDDVRQIAPGQTGVIADYNYKLPDDFMGCVLAWNTRNIDATSTGNYNYK |
| receptor binding | YRYLRHGKLRPFERDISNVPFSPDGKPCTPPALNCYWPLNDYGFYTTTGIGYQPYRVVVLSFELLNA |
| domain (SEQ ID | PATVCGPKLSTDLIKNQCVNF |
| NO: 2) | |
| SARS-CoV-1 | MYSFVSEETGTLIVNSVLLFLAFVVFLLVTLAILTALRLCAYCCNIVNVSLVKPTVYVYSRVKNLNS |
| Envelope Protein | SEGVPDLLV |
| (SEQ ID NO: 3) | |
| SARS-CoV-1 | MADNGTITVEELKQLLEQWNLVIGFLFLAWIMLLQFAYSNRNRFLYIIKLVFLWLLWPVTLACFVLA |
| Membrane | AVYRINWVTGGIAIAMACIVGLMWLSYFVASFRLFARTRSMWSFNPETNILLNVPLRGTIVTRPLME |
| Protein (SEQ ID | SELVIGAVIIRGHLRMAGHSLGRCDIKDLPKEITVATSRTLSYYKLGASQRVGTDSGFAAYNRYRIG |
| NO: 4) | NYKLNTDHAGSNDNIALLVQ |
| SARS-CoV-1 | MSDNGPQSNQRSAPRITFGGPTDSTDNNQNGGRNGARPKQRRPQGLPNNTASWFTALTQHGKEELRF |
| Nucleocapsid | PRGQGVPINTNSGPDDQIGYYRRATRRVRGGDGKMKELSPRWYFYYLGTGPEASLPYGANKEGIVWV |
| Protein (SEQ ID | ATEGALNTPKDHIGTRNPNNNAATVLQLPQGTTLPKGFYAEGSRGGSQASSRSSSRSRGNSRNSTPG |
| NO: 5) | SSRGNSPARMASGGGETALALLLLDRLNQLESKVSGKGQQQQGQTVTKKSAAEASKKPRQKRTATKQ |
| | YNVTQAFGRRGPEQTQGNFGDQDLIRQGTDYKHWPQIAQFAPSASAFFGMSRIGMEVTPSGTWLTYH |
| | GAIKLDDKDPQFKDNVILLNKHIDAYKTFPPTEPKKDKKKKTDEAQPLPQRQKKQPTVTLLPAADMD |
| | DFSRQLQNSMSGASADSTQA |
| SARS-CoV-2 | MFVFLVLLPLVSSQCVNLTTRTQLPPAYTNSFTRGVYYPDKVFRSSVLHSTQDLFLPFFSNVTWFHA |
| Spike Protein | IHVSGTNGTKRFDNPVLPFNDGVYFASTEKSNIIRGWIFGTTLDSKTQSLLIVNNATNVVIKVCEFQ |
| (SEQ ID NO: 6) | FCNDPFLGVYYHKNNKSWMESEFRVYSSANNCTFEYVSQPFLMDLEGKQGNFKNLREFVFKNIDGYF |
| | KIYSKHTPINLVRDLPQGFSALEPLVDLPIGINITRFQTLLALHRSYLTPGDSSSGWTAGAAAYYVG |
| | YLQPRTFLLKYNENGTITDAVDCALDPLSETKCTLKSFTVEKGIYQTSNFRVQPTESIVRFPNITNL |
| | CPFGEVFNATRFASVYAWNRKRISNCVADYSVLYNSASFSTFKCYGVSPTKLNDLCFTNVYADSFVI |
| | RGDEVRQIAPGQTGKIADYNYKLPDDFTGCVIAWNSNNLDSKVGGNYNYLYRLFRKSNLKPFERDIS |
| | TEIYQAGSTPCNGVEGFNCYFPLQSYGFQPTNGVGYQPYRVVVLSFELLHAPATVCGPKKSTNLVKN |
| | KCVNFNFNGLTGTGVLTESNKKFLPFQQFGRDIADTTDAVRDPQTLEILDITPCSFGGVSVITPGTN |
| | TSNQVAVLYQDVNCTEVPVAIHADQLTPTWRVYSTGSNVFQTRAGCLIGAEHVNNSYECDIPIGAGI |
| | CASYQTQTNSPRRARSVASQSIIAYTMSLGAENSVAYSNNSIAIPTNFTISVTTEILPVSMTKTSVD |
| | CTMYICGDSTECSNLLLQYGSFCTQLNRALTGIAVEQDKNTQEVFAQVKQIYKTPPIKDFGGFNFSQ |
| | ILPDPSKPSKRSFIEDLLFNKVTLADAGFIKQYGDCLGDIAARDLICAQKFNGLTVLPPLLTDEMIA |
| | QYTSALLAGTITSGWTFGAGAALQIPFAMQMAYRFNGIGVTQNVLYENQKLIANQFNSAIGKIQDSL |
| | SSTASALGKLQDVVNQNAQALNTLVKQLSSNFGAISSVLNDILSRLDKVEAEVQIDRLITGRLQSLQ |
| | TYVTQQLIRAAEIRASANLAATKMSECVLGQSKRVDFCGKGYHLMSFPQSAPHGVVFLHVTYVPAQE |
| | KNFTTAPAICHDGKAHFPREGVFVSNGTHWFVTQRNFYEPQIITTDNTFVSGNCDVVIGIVNNTVYD |
| | PLQPELDSFKEELDKYFKNHTSPDVDLGDISGINASVVNIQKEIDRLNEVAKNLNESLIDLQELGKY |
| | EQYIKWPWYIWLGFIAGLIAIVMVTIMLCCMTSCCSCLKGCCSCGSCCKFDEDDSEPVLKGVKLHYT |
| SARS-CoV-2 | RVQPTESIVRFPNITNLCPFGEVFNATRFASVYAWNRKRISNCVADYSVLYNSASFSTFKCYGVSPT |
| Spike Protein | KLNDLCFTNVYADSFVIRGDEVRQIAPGQTGKIADYNYKLPDDFTGCVIAWNSNNLDSKVGGNYNYL |
| receptor binding | YRLFRKSNLKPFERDISTEIYQAGSTPCNGVEGFNCYFPLQSYGFQPTNGVGYQPYRVVVLSFELLH |
| domain (SEQ ID | APATVCGPKKSTNLVKNKCVNF |
| NO: 7) | |
| SARS-CoV-2 | MYSFVSEETGTLIVNSVLLFLAFVVFLLVTLAILTALRLCAYCCNIVNVSLVKPSFYVYSRVKNLNS |
| Envelope Protein | SRVPDLLV |
| (SEQ ID NO: 8) | |
| SARS-CoV-2 | MADSNGTITVEELKKLLEQWNLVIGFLFLTWICLLQFAYANRNRFLYIIKLIFLWLLWPVTLACFVL |
| Membrane | AAVYRINWITGGIAIAMACLVGLMWLSYFIASFRLFARTRSMWSFNPETNILLNVPLHGTILTRPLL |
| Protein (SEQ ID | ESELVIGAVILRGHLRIAGHHLGRCDIKDLPKEITVATSRTLSYYKLGASQRVAGDSGFAAYSRYRI |
| NO: 9) | GNYKLNTDHSSSSDNIALLVQ |
| SARS-CoV-2 | MSDNGPQNQRNAPRITFGGPSDSTGSNQNGERSGARSKQRRPQGLPNNTASWFTALTQHGKEDLKFP |
| Nucleocapsid | RGQGVPINTNSSPDDQIGYYRRATRRIRGGDGKMKDLSPRWYFYYLGTGPEAGLPYGANKDGIIWVA |
| Protein (SEQ ID | TEGALNTPKDHIGTRNPANNAAIVLQLPQGTTLPKGFYAEGSRGGSQASSRSSSRSRNSSRNSTPGS |
| NO: 10) | SRGTSPARMAGNGGDAALALLLLDRLNQLESKMSGKGQQQQGQTVTKKSAAEASKKPRQKRTATKAY |
| | NVTQAFGRRGPEQTQGNFGDQELIRQGTDYKHWPQIAQFAPSASAFFGMSRIGMEVTPSGTWLTYTG |
| | AIKLDDKDPNFKDQVILLNKHIDAYKTFPPTEPKKDKKKKADETQALPQRQKKQQTVTLLPAADLDD |
| | FSKQLQQSMSSADSTQA |
| MERS-CoV | MIHSVFLLMFLLTPTESYVDVGPDSVKSACIEVDIQQTFFDKTWPRPIDVSKADGIIYPQGRTYSNI |
| Spike protein | TITYQGLFPYQGDHGDMYVYSAGHATGTTPQKLFVANYSQDVKQFANGFVVRIGAAANSTGTVIISP |
| (SEQ ID NO: 11) | STSATIRKIYPAFMLGSSVGNFSDGKMGRFFNHTLVLLPDGCGTLLRAFYCILEPRSGNHCPAGNSY |
| | TSFATYHTPATDCSDGNYNRNASLNSFKEYFNLRNCTFMYTYNITEDEILEWFGITQTAQGVHLFSS |
| | RYVDLYGGNMFQFATLPVYDTIKYYSIIPHSIRSIQSDRKAWAAFYVYKLQPLTFLLDFSVDGYIRR |
| | AIDCGFNDLSQLHCSYESFDVESGVYSVSSFEAKPSGSVVEQAEGVECDFSPLLSGTPPQVYNFKRL |
| | VFTNCNYNLTKLLSLFSVNDFTCSQISPAAIASNCYSSLILDYFSYPLSMKSDLSVSSAGPISQFNY |
| | KQSFSNPTCLILATVPHNLTTITKPLKYSYINKCSRLLSDDRTEVPQLVNANQYSPCVSTVPSTVWE |
| | DGDYYRKQLSPLEGGGWLVASGSTVAMTEQLQMGFGITVQYGTDTNSVCPKLEFANDTKIASQLGNC |
| | VEYSLYGVSGRGVFQNCTAVGVRQQRFVYDAYQNLVGYYSDDGNYYCLRACVSVPVSVIYDKETKTH |
| | ATLFGSVACEHISSTMSQYSRSTRSMLKRRDSTYGPLQTPVGCVLGLVNSSLFVEDCKLPLGQSLCA |
| | LPDTPSTLTPRSVRSVPGEMRLASIAFNHPIQVDQLNSSYFKLSIPTNFSFGVTQEYIQTTIQKVTV |
| | DCKQYVCNGFQKCEQLLREYGQFCSKINQALHGANLRQDDSVRNLFASVKSSQSSPIIPGFGGDFNL |
| | TLLEPVSISTGSRSARSAIEDLLFDKVTIADPGYMQGYDDCMQQGPASARDLICAQYVAGYKVLPPL |
| | MDVNMEAAYTSSLLGSIAGVGWTAGLSSFAAIPFAQSIFYRLNGVGITQQVLSENQKLIANKENQAL |
| | GAMQTGFTTTNEAFRKVQDAVNNNAQALSKLASELSNTFGAISASIGDIIQRLDVLEQDAQIDRLIN |
| | GRLTTLNAFVAQQLVRSESAALSAQLAKDKVNECVKAQSKRSGFCGQGTHIVSFVVNAPNGLYFMHV |
| | GYYPSNHIEVVSAYGLCDAANPTNCIAPVNGYFIKTNNTRIVDEWSYTGSSFYAPEPITSLNTKYVA |
| | PQVTYQNISTNLPPPLLGNSTGIDFQDELDEFFKNVSTSIPNFGSLTQINTTLLDLTYEMLSLQQVV |
| | KALNESYIDLKELGNYTYYNKWPWYIWLGFIAGLVALALCVFFILCCTGCGTNCMGKLKCNRCCDRY |
| | EEYDLEPHKVHVH |
| MERS-CoV | ECDFSPLLSGTPPQVYNFKRLVFTNCNYNLTKLLSLFSVNDFTCSQISPAAIASNCYSSLILDYFSY |
| Spike protein | PLSMKSDLSVSSAGPISQFNYKQSFSNPTCLILATVPHNLTTITKPLKYSYINKC |
| receptor binding | |
| domain (SEQ ID | |
| NO: 12) | |
| MERS-CoV | MLPFVQERIGLFIVNFFIFTVVCAITLLVCMAFLTATRLCVQCMTGFNTLLVQPALYLYNTGRSVYV |
| Envelope protein | KFQDSKPPLPPDEWV |
| (SEQ ID NO: 13) | |
| MERS-CoV | MSNMTQLTEAQIIAIIKDWNFAWSLIFLLITIVLQYGYPSRSMTVYVFKMFVLWLLWPSSMALSIFS |
| Membrane | AVYPIDLASQIISGIVAAVSAMMWISYFVQSIRLFMRTGSWWSFNPETNCLLNVPFGGTTVVRPLVE |
| protein (SEQ ID | DSTSVTAVVTNGHLKMAGMHFGACDYDRLPNEVTVAKPNVLIALKMVKRQSYGTNSGVAIYHRYKAG |
| NO: 14) | NYRSPPITADIELALLRA |
| MERS-CoV | MASPAAPRAVSFADNNDITNTNLSRGRGRNPKPRAAPNNTVSWYTGLTQHGKVPLTFPPGQGVPLNA |
| Nucleocapsid | NSTPAQNAGYWRRQDRKINTGNGIKQLAPRWYFYYTGTGPEAALPFRAVKDGIVWVHEDGATDAPST |
| protein (SEQ ID | FGTRNPNNDSAIVTQFAPGTKLPKNFHIEGTGGNSQSSSRASSLSRNSSRSSSQGSRSGNSTRGTSP |
| NO: 15) | GPSGIGAVGGDLLYLDLLNRLQALESGKVKQSQPKVITKKDAAAAKNKMRHKRTSTKSFNMVQAFGL |
| | RGPGDLQGNFGDLQLNKLGTEDPRWPQIAELAPTASAFMGMSQFKLTHQNNDDHGNPVYFLRYSGAI |
| | KLDPKNPNYNKWLELLEQNIDAYKTFPKKEKKQKAPKEESTDQMSEPPKEQRVQGSITQRTRTRPSV |
| | QPGPMIDVNTD |

In some embodiments, the antigen is a wild type (i.e., “native”) antigen. In some embodiments, the antigen is a wild type protein or polypeptide antigen. In some embodiments, the antigen is a polypeptide variant to a wild type protein or polypeptide antigen. The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, the polypeptide variant is a variant of high consequence (VOHC), a variant of concern (VOC), a variant of interest (VOI), or a variant being monitored (VBM). These four categories are used to identify the degree of risk a polypeptide variant poses to public health. For example, a variant of high consequence (VOHC) refers to a variant that is associated with severe consequences, such as significantly increased morbidity or mortality, and may have the potential to evade immunity or undermine public health measures, while a variant of concern (VOC) refers to a variant that exhibits increased transmissibility, virulence, reduced effectiveness of treatments, and/or decreased vaccine efficacy as compared to other circulating strains. In some embodiments, the polypeptide variant possesses at least 50% identity to a native or reference sequence. In some embodiments, the polypeptide variant shares at least 60%, at least 70%, at least 80%, at least 90%, at least 95%, or at least 99% identity with a native or reference sequence.

In some embodiments, the polypeptide variant comprises substitutions, insertions, and/or deletions. In some embodiments, the polypeptide variant encompasses covalent variants and derivatives. The term “derivative” is used synonymously with the term “variant” but generally refers to a molecule that has been modified and/or changed in any way relative to a reference molecule or starting molecule.

In some embodiments, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

In some embodiments, the polypeptide variants comprise at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule. In some embodiments, the antigen is a polypeptide that includes 2, 3, 4, 5, 6, 7, 8, 9, 10, or more substitutions compared to a reference protein.

In some embodiments, the substitution is a conservative amino acids substitution. The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In some embodiments, protein fragments, functional protein domains, and homologous proteins are used as antigens in accordance with the present disclosure. For example, an antigen may comprise any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to a reference protein (e.g., a protein from a microbial pathogen) herein can be utilized in accordance with the disclosure.

In some embodiments, the antigen comprises more than one immunogenic proteins or polypeptides (e.g., 2, 3, 4, 5, 6, 7, 8, 9, 10, or more). In some embodiments, the more than one immunogenic proteins or polypeptides are derived from one protein (e.g., different fragments or one protein). In some embodiments, the more than one immunogenic proteins or polypeptides are derived from multiple proteins (e.g., from 2, 3, 4, 5, 6, 7, 8, 9, 10, or more proteins).

In some embodiments, an immunogenic composition of the present disclosure comprises a nucleic acid encoding an antigen (e.g., as described herein). In some embodiments, the antigen comprises a nucleic acid encoding an immunogenic protein or polypeptide. The term “nucleic acid” or “polynucleotide,” in its broadest sense, includes any compound and/or substance that comprises a polymer of nucleotides. Nucleic acids encoding immunogenic proteins or polypeptides typically comprise an open reading frame (ORF), and one or more regulatory sequences. Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, the nucleic acid encoding an antigen (e.g., immunogenic protein or polypeptide) is DNA (e.g., an expression vector for an immunogenic protein or polypeptide). In some embodiments, the nucleic acid encoding an antigen (e.g., immunogenic protein or polypeptide) is RNA (e.g., a messenger RNA). A “messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ, or ex vivo. The basic components of an mRNA molecule typically include at least one coding region, a 5′ untranslated region (UTR), a 3′ UTR, a 5′ cap and a poly-A tail.

In some embodiments, the coding region of the nucleic acid (e.g., DNA or RNA) encoding an antigen (e.g., an immunogenic protein or polypeptide) is codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding an immunogenic protein or polypeptide). In some embodiments, a codon optimized sequence shares less than 90% sequence identity to a naturally-occurring or wild-type sequence (e.g., a naturally-occurring or wild-type mRNA sequence encoding an immunogenic protein or polypeptide). In some embodiments, a codon optimized sequence shares less than 85% sequence identity to a naturally-occurring or wild-type sequence (e.g., a naturally-occurring or wild-type mRNA sequence encoding an immunogenic protein or polypeptide). In some embodiments, a codon optimized sequence shares less than 80% sequence identity to a naturally-occurring or wild-type sequence (e.g., a naturally-occurring or wild-type mRNA sequence encoding an immunogenic protein or polypeptide). In some embodiments, a codon optimized sequence shares less than 75% sequence identity to a naturally-occurring or wild-type sequence (e.g., a naturally-occurring or wild-type mRNA sequence encoding an immunogenic protein or polypeptide).

In some embodiments, the nucleic acid encoding an antigen (e.g., immunogenic protein or polypeptide) comprises one or more chemical modifications. The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population.

In some embodiments, the nucleic acids are antigens or encode antigens. In some embodiments, the nucleic acids (e.g., DNA or RNA) comprise various (more than one) different modifications. In some embodiments, a particular region of a nucleic acid (e.g., DNA or RNA) contains one, two or more (optionally different) nucleoside or nucleotide modifications. In some embodiments, a modified nucleic acid (e.g., DNA or RNA), introduced to a cell or organism, exhibits reduced degradation in the cell or organism, respectively, relative to an unmodified nucleic acid. In some embodiments, a modified nucleic acid (e.g., DNA or RNA), introduced into a cell or organism, may exhibit reduced immunogenicity in the cell or organism, respectively (e.g., a reduced innate response).

Modified nucleic acids (e.g., DNA or RNA) may comprise modifications that are naturally-occurring and/or non-naturally-occurring. Polynucleotides may include any useful modification, for example, of a sugar, a nucleobase, or an internucleoside linkage (e.g., to a linking phosphate, to a phosphodiester linkage or to the phosphodiester backbone). Modified nucleic acids (e.g., DNA or RNA), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

In some embodiments, a chemically modified nucleic acid comprises one or more modified nucleosides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside and a (one or more) phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

In some embodiments, a modified nucleobase is a modified uridine. Exemplary nucleobases and nucleosides having a modified uridine include 5-cyano uridine, and 4′-thio uridine. In some embodiments, a modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A). In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

In some embodiments, the immunogenic composition described herein comprise a nucleic acid encoding an antigen, wherein the nucleic acid encodes a Beta coronavirus protein or polypeptide. In some embodiments, the immunogenic composition described herein comprise a nucleic acid encoding an antigen, wherein the nucleic acid encodes a Beta coronavirus spike protein, a Beta coronavirus nucleocapsid protein, a Beta coronavirus membrane protein, a Beta coronavirus envelope protein, or an immunogenic fragment thereof (e.g., a Beta coronavirus spike protein receptor binding domain (RBD)). In some embodiments, the nucleic acid encodes a MERS-CoV spike protein, a MERS-CoV nucleocapsid protein, a MERS-CoV membrane protein, a MERS-CoV envelope protein, or an immunogenic fragment thereof (e.g., a MERS-CoV spike protein RBD). In some embodiments, the nucleic acid encodes a SARS-CoV-1 spike protein, a SARS-CoV-1 nucleocapsid protein, a SARS-CoV-1 membrane protein, a SARS-CoV-1 envelope protein, or an immunogenic fragment thereof (e.g., a SARS-CoV-1 spike protein RBD). In some embodiments, the nucleic acid encodes a SARS-CoV-2 spike protein, a SARS-CoV-2 nucleocapsid protein, a SARS-CoV-2 membrane protein, a SARS-CoV-2 envelope protein, or an immunogenic fragment thereof (e.g., a SARS-CoV-2 spike protein RBD).

Polypeptide or polynucleotide molecules of the present disclosure may share a certain degree of sequence similarity or identity with reference molecules (e.g., reference polypeptides or reference polynucleotides), for example, wild-type molecules. The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

The term “identity” refers to the overall relatedness between polymeric molecules, for example, between polynucleotide molecules (e.g., DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In some embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988). Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

In some embodiments, the Beta coronavirus antigen in the immunogenic composition (e.g., vaccine composition) described herein comprises a protein having an amino acid sequence that is at least 70% (e.g., at least 70%, at least 75%, at least 80%, at least 85%, at least 90%, at least 95%, or at least 99%) identical to any one of SEQ ID NOs: 1-15. In some embodiments, the Beta coronavirus antigen in the immunogenic composition (e.g., vaccine composition) described herein comprises a protein having an amino acid sequence that is 70%, 75%, 80%, 85%, 90%, 95%, or 99% identical to any one of SEQ ID NOs: 1-15. In some embodiments, the Beta coronavirus antigen in the immunogenic composition (e.g., vaccine composition) described herein comprises a protein comprising the amino acid sequence of any one of SEQ ID NO: 1-15.

In some embodiments, the nucleic acid encoding a Beta coronavirus antigen in the immunogenic composition (e.g., vaccine composition) described herein comprises a nucleic acid (e.g., DNA or RNA, such as mRNA) encoding a protein having an amino acid sequence that is at least 70% (e.g., at least 70%, at least 75%, at least 80%, at least 85%, at least 90%, at least 95%, or at least 99%) identical to any one of SEQ ID NOs: 1-15. In some embodiments, the nucleic acid encoding a Beta coronavirus antigen in the immunogenic composition (e.g., vaccine composition) described herein comprises a nucleic acid (e.g., DNA or RNA, such as mRNA) encoding a protein having an amino acid sequence that is 70%, 75%, 80%, 85%, 90%, 95%, or 99% identical to any one of SEQ ID NOs: 1-15. In some embodiments, the nucleic acid encoding a Beta coronavirus antigen in the immunogenic composition (e.g., vaccine composition) described herein comprises a nucleic acid (e.g., DNA or RNA, such as mRNA) encoding a protein comprising the amino acid sequence of any one of SEQ ID NO: ID NOs: 1-15.

In some embodiments, an adjuvantation system of the present disclosure is admixed with a nucleic acid encoding an antigen. In some embodiments, the weight ratio of the nucleic acid to the adjuvantation system is between 1:1 and 1:100. In some embodiments, the weight ratio of the nucleic acid to the adjuvantation system is 1:10. In some embodiments, the weight ratio of the nucleic acid to the adjuvantation system is 1:100.

In some embodiments, the immunogenic composition comprises a mannan (e.g., a plant mannan or a fungal mannan), an aluminum salt, and a nucleic acid encoding an antigen. In some embodiments, the immunogenic composition comprises a plant mannan (e.g., a konjac glucomannan), an aluminum salt, and a nucleic acid encoding an antigen. In some embodiments, the immunogenic composition comprises a fungal mannan (e.g., a C. albicans mannan), an aluminum salt, and a nucleic acid encoding an antigen. In some embodiments, the immunogenic composition comprises a nucleic acid encoding an antigen and an adjuvantation system, wherein the adjuvantation system comprises aluminum salt and konjac glucomannan. In some embodiments, the immunogenic composition comprises a nucleic acid encoding an antigen and an adjuvantation system at a 1:60 weight ratio of nucleic acid to adjuvantation system, wherein the adjuvantation system comprises aluminum salt and konjac glucomannan at a 1:4 weight ratio of aluminum salt to konjac glucomannan. In some embodiments, the immunogenic composition comprises a nucleic acid encoding an antigen and an adjuvantation system at a 1:100 weight ratio of nucleic acid to adjuvantation system, wherein the adjuvantation system comprises aluminum salt and konjac glucomannan at a 1:4 weight ratio of aluminum salt to konjac glucomannan. In some embodiments, the immunogenic composition comprises a nucleic acid encoding an antigen and an adjuvantation system, wherein the adjuvantation system comprises aluminum salt and C. albicans mannan. In some embodiments, the immunogenic composition comprises a nucleic acid encoding an antigen and an adjuvantation system at a 1:100 weight ratio of nucleic acid to adjuvantation system, wherein the adjuvantation system comprises aluminum salt and C. albicans mannan at a 1:5 weight ratio of aluminum salt to C. albicans mannan.

In some embodiments, an adjuvantation system of the present is admixed with an antigen. In some embodiments, the weight ratio of the antigen to the adjuvantation system is between 1:1 and 1:100. In some embodiments, the weight ratio of the antigen to the adjuvantation system is 1:1. In some embodiments, the weight ratio of the antigen to the adjuvantation system is 1:10. In some embodiments, the weight ratio of the antigen to the adjuvantation system is 1:100.

In some embodiments, the immunogenic composition comprises a plant mannan (e.g., a konjac glucomannan), an aluminum salt, and an antigen. In some embodiments, the immunogenic composition comprises an antigen and an adjuvantation system, wherein the adjuvantation system comprises aluminum salt and konjac glucomannan. In some embodiments, the immunogenic composition comprises an antigen and an adjuvantation system at a 1:60 weight ratio of antigen to adjuvantation system, wherein the adjuvantation system comprises aluminum salt and konjac glucomannan at a 1:4 weight ratio of aluminum salt to konjac glucomannan. In some embodiments, the immunogenic composition comprises an antigen and an adjuvantation system at a 1:100 weight ratio of antigen to adjuvantation system, wherein the adjuvantation system comprises aluminum salt and konjac glucomannan at a 1:4 weight ratio of aluminum salt to konjac glucomannan.

Adjuvants and adjuvantation systems as described herein are used in immunogenic compositions (e.g., a vaccine composition). The terms “vaccine composition” and “vaccine” are used interchangeably herein. An “immunogenic composition” is a composition that activates or enhances a subject's immune response to an antigen after the vaccine is administered to the subject. Vaccine compositions are a type of immunogenic composition. In some embodiments, the present disclosure provides a vaccine comprising an immunogenic composition described herein and a pharmaceutically acceptable excipient.

In some embodiments, the immunogenic composition (e.g., vaccine composition) described herein further comprises a pharmaceutically-acceptable excipient. In some embodiments, the immunogenic compositions (e.g., vaccine composition) described herein are formulated for administration to a subject. In some embodiments, the immunogenic compositions (e.g., vaccine composition) described herein are formulated or administered in combination with one or more pharmaceutically-acceptable excipients. In some embodiments, the immunogenic compositions (e.g., vaccine composition) described herein are formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo.

The phrase “pharmaceutically-acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable excipient” means a pharmaceutically-acceptable material, composition or vehicle, such as a liquid or solid filler, diluent, carrier, solvent or encapsulating material, involved in carrying or transporting the subject agents from one organ, or portion of the body, to another organ, or portion of the body. Each excipient must be “acceptable” in the sense of being compatible with the other ingredients of the formulation and not injurious to the tissue of the patient (e.g., physiologically compatible, sterile, physiologic pH, etc.). The term “excipient” denotes an organic or inorganic ingredient, natural or synthetic, with which the active ingredient is combined to facilitate the application. The components of the immunogenic compositions (e.g., vaccine composition) described herein also are capable of being co-mingled with the molecules of the present disclosure, and with each other, in a manner such that there is no interaction which would substantially impair the desired pharmaceutical efficacy. Some examples of materials which can serve as pharmaceutically-acceptable excipients include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (18) ethyl alcohol; (19) pH buffered solutions; (20) polyesters, polycarbonates and/or polyanhydrides; (21) bulking agents, such as polypeptides and amino acids (22) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA vaccines (e.g., for transplantation into a subject), hyaluronidase, nanoparticle mimics and combinations thereof.

In some embodiments, the immunogenic composition (e.g., vaccine composition) is formulated in an aqueous solution. In some embodiments, the immunogenic composition (e.g., vaccine composition) is formulated in a nanoparticle. In some embodiments, the immunogenic composition (e.g., vaccine composition) is formulated in a lipid nanoparticle. In some embodiments, the immunogenic composition (e.g., vaccine composition) is formulated in a lipid-polycation complex, referred to as a lipid nanoparticle. In some embodiments, where the immunogenic composition (e.g., vaccine composition) is formulated in a nanoparticle (e.g., a lipid nanoparticle), the nanoparticle has a diameter between 400 nm-1600 nm. In some embodiments, where the immunogenic composition (e.g., vaccine composition) is formulated in a nanoparticle (e.g., a lipid nanoparticle), the nanoparticle has a diameter between 200 nm-500 nm, 400 nm-500 nm, 400 nm-750 nm, 400 nm-1000 nm, 400 nm-1250 nm, 400-1500 nm, 500 nm-750 nm, 500 nm-1000 nm, 500 nm-1250 nm, 500 nm-1600 nm, 750 nm-1000 nm, 750 nm-1250 nm, 750 nm-1500 nm, 750 nm-1600 nm, 1000 nm-1250 nm, 1000 nm-1600 nm, or 1250 nm-1600 nm. “Between” as used in reference to a range refers to an inclusive range. In some embodiments, the nanoparticle is at least 200 nm in diameter. In some embodiments, the nanoparticle is at least 400 nm in diameter. In some embodiments, the nanoparticle is less than 1000 nm in diameter. In some embodiments, this disclosure provides a plurality of nanoparticles as described herein with an average diameter between 300-500 nm, 350-450 nm, 500-1000 nm or 500-700 nm.

The formation of the lipid nanoparticle may be accomplished by methods known in the art, for example as described in U.S. Pub. No. 20120178702. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818. In some embodiments, the immunogenic composition (e.g., vaccine composition) is formulated in a lipid nanoparticle that includes a non-cationic lipid such as, but not limited to, cholesterol or dioleoyl phosphatidylethanolamine (DOPE).

In some embodiments, a vaccine formulation described herein is a nanoparticle that comprises at least one lipid (termed a “lipid nanoparticle” or “LNP”). The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof. Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578.

In some embodiments, the immunogenic composition (e.g., vaccine composition) is formulated in a liposome. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. In some embodiments, the compositions described herein, when formulated in a liposome, comprise two distinct particle sizes, wherein one size is associated with the liposome and the other size is associated with the adjuvantation system. In some embodiments, the liposomes comprise a nucleic acid. In some embodiments, the adjuvantation system comprises alum-mannan complex. In some embodiments, the particle size is analyzed using dynamic light scattering technic. In some embodiments, the liposomes remain intact and show minimal physicochemical interactions with the adjuvantation system.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products. As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372. In some embodiments, the immunogenic composition (e.g., vaccine composition) described herein may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120) and liposomes which may deliver small molecule drugs such as, but not limited to, DOXIL® from Janssen Biotech, Inc. (Horsham, PA).

In some embodiments, the immunogenic composition may be formulated in a water-in-oil emulsion comprising a continuous hydrophobic phase in which the hydrophilic phase is dispersed. As a non-limiting example, the emulsion may be made by the methods described in International Publication No. WO201087791.

In some embodiments, the compositions described herein are formulated in nanoparticles, liposomes, emulsions, or combinations thereof. In some embodiments, the nucleic acid is outside the nanoparticles. In some embodiments, the nucleic acid in within the nanoparticles. In some embodiments, the nucleic acid is a DNA, an RNA or a combination thereof. In some embodiments, the mannan is within the nanoparticles. In some embodiments, the mannan is outside the nanoparticles. In some embodiments, the mannan comprises a linear polysaccharide or a branched polysaccharide. In some embodiments, the mannan is within lipid membrane of the lipid nanoparticle. In some embodiments, the mannan is within the lipid nanoparticles. In some embodiments, the nucleic acid is administered naked.

In some embodiments, immunogenic compositions (e.g., vaccine composition) comprise at least one additional active substances, such as, for example, a therapeutically-active substance, a prophylactically-active substance, or a combination of both. Immunogenic compositions (e.g., vaccine composition) may be sterile, pyrogen-free or both sterile and pyrogen-free. General considerations in the formulation and/or manufacture of pharmaceutical agents, such as immunogenic compositions (e.g., vaccine composition), may be found, for example, in Remington: The Science and Practice of Pharmacy 21st ed., Lippincott Williams & Wilkins, 2005.

Formulations of the immunogenic composition (e.g., vaccine composition) described herein may be prepared by any method known or hereafter developed in the art of pharmacology. In general, such preparatory methods include the step of bringing the immunogenic composition into association with an excipient and/or one or more other accessory ingredients, and then, if necessary and/or desirable, dividing, shaping and/or packaging the product into a desired single- or multi-dose unit.

Relative amounts of the immunogenic composition, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The immunogenic composition (e.g., vaccine composition) described herein may conveniently be presented in unit dosage form and may be prepared by any of the methods well-known in the art of pharmacy. The term “unit dose” when used in reference to an immunogenic composition (e.g., vaccine composition) described herein refers to physically discrete units suitable as unitary dosage for the subject, each unit containing a predetermined quantity of active material calculated to produce the desired therapeutic effect in association with the required diluent; i.e., excipient.

In some embodiments, the immunogenic compositions (e.g., vaccine composition) described herein are formulated for administration to a subject. In some embodiments, the immunogenic compositions (e.g., vaccine compositions) described herein are formulated with a pharmaceutically-acceptable excipient for administration to a subject. The formulation of the immunogenic compositions (e.g., vaccine composition) described herein may be dependent upon the route of administration. Injectable preparations suitable for parenteral administration, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the immunogenic composition (e.g., vaccine composition) described herein can be formulated into ointments, salves, gels, or creams, as is generally known in the art. Topical administration can utilize transdermal delivery systems well known in the art. An example is a dermal patch. Immunogenic compositions suitable for oral administration may be presented as discrete units, such as capsules, tablets, lozenges, each containing a predetermined amount of the anti-inflammatory agent. Other immunogenic compositions suitable for oral administration include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the immunogenic composition (e.g., vaccine composition) described herein used for administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The cyclic Psap peptide and/or the composition or immunogenic composition (e.g., vaccine composition) described herein ordinarily will be stored in lyophilized form or as an aqueous solution if it is highly stable to thermal and oxidative denaturation. The pH of the preparations typically will be about from 6 to 8, although higher or lower pH values can also be appropriate in certain instances. The chimeric constructs of the present disclosure can be used as vaccines by conjugating to soluble immunogenic carrier molecules. Suitable carrier molecules include protein, including keyhole limpet hemocyanin, which is a preferred carrier protein. The chimeric construct can be conjugated to the carrier molecule using standard methods. (Hancock et al., “Synthesis of Peptides for Use as Immunogens,” in Methods in Molecular Biology: Immunochemical Protocols, Manson (ed.), pages 23-32 (Humana Press 1992)).

The prophylactic or therapeutic use of the immunogenic composition (e.g., vaccine compositions) described herein is also within the scope of the present disclosure. In some embodiments, the immunogenic compositions (e.g., vaccine composition) described herein are used in methods of vaccinating a subject by prophylactically administering to the subject an effective amount of the immunogenic composition (e.g., vaccine composition) described herein. “Vaccinating a subject” refers to a process of administering an immunogen (e.g., immunogenic composition), to the subject in an amount effective to increase or activate an immune response against the antigen (e.g., a Beta coronavirus antigen), and thus against the pathogen (e.g., Beta coronavirus). In some embodiments, the term “vaccinating a subject” does not require the creation of complete immunity against the virus. In some embodiments, the term “vaccinating a subject” encompasses a clinically favorable enhancement of an immune response toward the viral antigen or pathogen. In some embodiments, vaccinating a subject reduces the risk of developing a viral infection, such as a Beta coronavirus (e.g., MERS-CoV, SARS-CoV-1, or SARS-CoV-2) infection, and diseases that occur as a result of viral infection, such as those caused by Beta coronavirus infection (e.g., MERS, SARS and/or COVID-19).

Other aspects of this disclosure provide, a method of stimulating immune cell recruitment in a subject in need thereof, the method comprising administering a composition comprising an adjuvantation system and a nucleic acid, wherein the adjuvantation system comprises a mannan. In some embodiments, the method recruits more immune cells relative to a subject treated with the composition that does not comprise mannan. In some embodiments, the method recruits more immune cells against viral antigen selected from L1 antigen, E6 antigen, E7 antigen, HBsAg, Epstein-Barr nuclear antigen, SV40 or combinations thereof.

Other aspects of the present disclosure provide methods of inducing an immune response against a pathogen in a subject in need thereof, the method comprising administering to the subject an effective amount of an antigen and an effective amount of an adjuvantation system (e.g., as described herein). Additional aspects of the present disclosure provides methods of inducing an immune response against a pathogen in a subject in need thereof, the method comprising administering to the subject an effective amount of a nucleic acid encoding an antigen and an effective amount of an adjuvantation system (e.g., as described herein).

In some embodiments, the present disclosure provides methods of inducing an immune response against a Beta coronavirus (e.g., MERS-CoV, SARS-CoV-1, or SARS-CoV-2) in a subject in need thereof, the method comprising administering to the subject an effective amount of a Beta coronavirus antigen and an effective amount of an adjuvantation system (e.g., as described herein). In some embodiments, the present disclosure provides methods of inducing an immune response against a Beta coronavirus (e.g., MERS-CoV, SARS-CoV-1, or SARS-CoV-2) in a subject in need thereof, the method comprising administering to the subject an effective amount of a nucleic acid encoding a Beta coronavirus antigen and an effective amount of an adjuvantation system, wherein the adjuvantation system comprises a plant mannan (e.g., a konjac glucomannan). In some embodiments, the present disclosure provides methods of inducing an immune response against a Beta coronavirus (e.g., MERS-CoV, SARS-CoV-1, or SARS-CoV-2) in a subject in need thereof, the method comprising administering to the subject an effective amount of a nucleic acid encoding a Beta coronavirus antigen and an effective amount of an adjuvantation system, wherein the adjuvantation system comprises a fungal mannan (e.g., a C. albicans mannan).

Other aspects of this disclosure provide, a method of draining lymph node in a subject in need thereof, the method comprising administering a composition comprising an adjuvantation system and a nucleic acid in a subject in need thereof, wherein the adjuvantation system comprises a mannan. In some embodiments, the subject is a mammalian subject.

A “subject” to which administration is contemplated refers to a human (i.e., male or female of any age group, e.g., pediatric subject (e.g., infant, child, or adolescent) or adult subject (e.g., young adult, middle-aged adult, or senior adult)), or a non-human animal. In some embodiments, the non-human animal is a mammal (e.g., primate, such as a cynomolgus monkey or a rhesus monkey), commercially relevant mammal (e.g., cattle, pig, horse, sheep, goat, cat, or dog), or bird (e.g., commercially relevant bird, such as chicken, duck, goose, or turkey). In some embodiments, the non-human animal is a fish, reptile, or amphibian. The non-human animal may be a male or female at any stage of development. The non-human animal may be a transgenic animal or a genetically engineered animal.

A “subject in need thereof” refers to a subject (e.g., a human subject or a non-human mammal) in need of treatment of infection (e.g., by a pathogen), such as infection by a Beta coronavirus (e.g., a subject having MERS, SARS or COVID-19), or in need of reducing the risk of developing an infection (e.g., by a pathogen), such as infection by a Beta coronavirus (e.g., MERS-CoV, SARS-CoV-1, or SARS-CoV-2). In some embodiments, administering the antigen or nucleic acid encoding the antigen, such as a Beta coronavirus (e.g., MERS-CoV, SARS-CoV-1, or SARS-CoV-2) antigen or nucleic acid encoding a Beta coronavirus antigen, and the adjuvantation system described herein to a subject having an infection, such as an infection by a Beta coronavirus (e.g., MERS-CoV, SARS-CoV-1, or SARS-CoV-2), treats (i.e., has a therapeutic use for) the infection (e.g., MERS, SARS, or COVID-19). In some embodiments, administering the antigen or nucleic acid encoding the antigen, such as a Beta coronavirus (e.g., MERS-CoV, SARS-CoV-1, or SARS-CoV-2) antigen or nucleic acid encoding a Beta coronavirus antigen, and the adjuvantation system described herein to a subject at risk of developing an infection, such as an infection by a Beta coronavirus (e.g., MERS-CoV, SARS-CoV-1, or SARS-CoV-2), reduces the likelihood (e.g., by 20%, 30%, 40%, 50%, 60%, 70%, 80%, 90%, 95%, 99% or more) of the subject developing the infection (prophylactic use).

In some embodiments, the subject is a human subject, e.g., a human neonate, infant, child, adult, or elderly. In some embodiments, the human subject has an undeveloped (e.g., an infant or a neonate), weak (an elderly), or compromised immune system. Immunocompromised subjects include, without limitation, subjects with primary immunodeficiency or acquired immunodeficiency such as those suffering from sepsis, HIV infection, and cancers, including those undergoing chemotherapy and/or radiotherapy. In some embodiments, the human subject has an underlying condition that renders them more susceptible to an infection, such as a Beta coronavirus (e.g., MERS-CoV, SARS-CoV-1, or SARS-CoV-2) infection. In some embodiments, the human subject is immunocompromised, has chronic lung disease, asthma, cardiovascular disease, cancer, obesity, diabetes, chronic kidney disease, and/or liver disease.

In some embodiments, the subject is a companion animal (i.e., a pet or service animal). The use of the immunogenic composition (e.g., vaccine composition) described herein in a veterinary vaccine is also within the scope of the present disclosure. “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., ferrets, pigs, rats, mice, guinea pigs, and hamsters), rabbits, or non-human primates.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. Prophylactic treatment refers to the treatment of a subject who is not and was not with a disease but is at risk of developing the disease or who was with a disease, is not with the disease, but is at risk of regression of the disease. In some embodiments, the subject is at a higher risk of developing the disease or at a higher risk of regression of the disease than an average healthy member of a population.

An “effective amount” of a composition described herein refers to an amount sufficient to elicit the desired biological response. An effective amount of a composition described herein may vary depending on such factors as the desired biological endpoint, the pharmacokinetics of the compound, the condition being treated, the mode of administration, and the age and health of the subject. In some embodiments, an effective amount is a therapeutically effective amount. In some embodiments, an effective amount is a prophylactic treatment. In some embodiments, an effective amount is the amount of a compound described herein in a single dose. In some embodiments, an effective amount is the combined amounts of a compound described herein in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a compound described herein, or a composition thereof (e.g., and immunogenic composition), in or on a subject. The immunogenic composition (e.g., vaccine composition) described herein may be administered systemically (e.g., via intravenous injection) or locally (e.g., via local injection). In some embodiments, the immunogenic composition (e.g., vaccine composition) described herein is administered orally, intravenously, topically, intranasally, or sublingually. Parenteral administration is also contemplated. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, the immunogenic composition (e.g., vaccine composition) described herein is administered intramuscularly. In some embodiments, the immunogenic composition (e.g., vaccine composition) described herein is administered prophylactically.

In some embodiments, the immunogenic composition (e.g., vaccine composition) is administered once or multiple times (e.g., 2, 3, 4, 5, or more times). Where an immunogenic composition (e.g., vaccine composition) is administered multiple times, the administration typically follows a homologous (e.g., using the same immunogenic composition for each administration) prime/boost vaccination schedule. For multiple administrations, the administrations may be done over a period of time (e.g., 1 week, 2 weeks, 3 weeks, 1 month, 2 months, 3 months, 4 months, 5 months, 6 months, 1 year, 2 years, 5 years, 10 years, or more than 10 years). In some embodiments, the immunogenic composition (e.g., vaccine composition) is administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later). In some embodiments, the immunogenic composition (e.g., vaccine composition) is administered three times (e.g., Days 0, 7, and 14; Days 0, 14, and 28; Days 0, 14 and 56; Days 0, 28, and 56).

Once administered, the immunogenic composition (e.g., vaccine composition) described herein elicits an immune response in the subject. In some embodiments, the immunogenic compositions (e.g., vaccine composition) described herein induces an immune response. In some embodiments, the immune response is type 1 immune response, characterized by production and secretion of IgE antibodies from B cells, vasodilation, and leukocyte extravasation. In some embodiments, the immune response is an innate immune response. In some embodiments, the immune response is an adaptive immune response specific to the antigen in the composition or vaccine. In some embodiments, the immunogenic composition (e.g., vaccine composition) described herein activates B cell immunity. In some embodiments, the immunogenic composition (e.g., vaccine composition) elicits production of antibodies (immunoglobulins, e.g., IgE, IgG, IgA, IgM, or sub-types thereof, e.g., IgG1, IgG2, IgG3, and IgG4) against the antigen. In some embodiments, the immunogenic composition (e.g., vaccine composition) activates cytotoxic T cells specific to the antigen. In some embodiments, the immunogenic composition (e.g., vaccine composition) elicits production of cytokines (e.g., chemokines, interferons, interleukins, etc.) by antigen-specific T cells.

In some embodiments, the immunogenic compositions (e.g., vaccine composition) described herein enhances the production of a cytokine (e.g., IL-1β, IL-6, TNF, pro-IL-1) in the subject. In some embodiments, the immunogenic composition (e.g., vaccine composition) described herein enhances the level of cytokines (e.g., IL-1β, IL-6, TNF, pro-IL-1β) is increased by at least 20%, compared to when the antigen or nucleic acid encoding the antigen is administered alone. For example, the immunogenic composition (e.g., vaccine composition) described herein may increase the level of cytokines (e.g., IL-1β, IL-6, TNF, pro-IL-1β) by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold, or more, compared to when the antigen or nucleic acid encoding the antigen is administered alone. In some embodiments, the immunogenic composition (e.g., vaccine composition) described herein may increase the level of cytokines (e.g., IL-1β, IL-6, TNF, pro-IL-1) by 20%, 30%, 40%, 50%, 60%, 70%, 80%, 90%, 100%, 2-fold, 5-fold, 10-fold, 100-fold, 1000-fold, or more, compared to when the antigen or nucleic acid encoding the antigen is administered alone.

In some embodiments, the immunogenic compositions (e.g., vaccine composition) described herein stimulates the subject's immune system to recognize the antigen (e.g., a Beta coronavirus antigen) as foreign, and enhances the subject's immune response if the subject is later exposed to a pathogen (e.g., Beta coronavirus). In some embodiments, an immunogenic composition (e.g., vaccine composition) is used to protect or treat an organism against a disease (e.g., MERS, SARS, and/or COVID-19). In some embodiments, the immunogenic compositions (e.g., vaccine compositions) described herein promote the activation of dendritic cell-associated C-type lectin 2 (Dectin-2) in the subject. In some embodiments, the immunogenic compositions (e.g., vaccine composition) described herein enhance the production of antigen-specific antibodies, compared to when the antigen or nucleic acid encoding the antigen is administered alone. In some embodiments, the antigen-specific antibody is of IgG, IgG1, or IgG2c type. In some embodiments, the immunogenic compositions (e.g., vaccine compositions) described herein prolong a protective effect in the subject against the antigen, compared to when the antigen or nucleic acid encoding the antigen is administered alone. In some embodiments, the immunogenic compositions (e.g., vaccine compositions) described herein enhance the recall response, compared to when the antigen of nucleic acid encoding the antigen is administered alone.

In some embodiments, the immunogenic compositions (e.g., vaccine compositions) described herein enhances the antigen-specific immune response against the antigen (e.g., a Beta coronavirus antigen, such as a MERS-CoV antigen, a SARS-CoV-1 antigen, or a SARS-CoV-2 antigen) or against the virus (e.g., a Beta coronavirus, such as MERS-CoV, SARS-CoV-1, or SARS-CoV-2), compared to when the antigen or nucleic acid encoding the antigen is administered alone. In some embodiments, the adjuvantation system enhances the production of antigen-specific antibody titers (e.g., by at least 20%) in the subject, compared to when the antigen or nucleic acid encoding the antigen is administered alone. For example, the adjuvantation system may enhance the production of antigen-specific antibody titers by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more in the subject compared to when the antigen or nucleic acid encoding the antigen is administered alone. In some embodiments, the adjuvantation system enhances the production of antigen-specific antibody titers by 20%, 20%, 40%, 50%, 60%, 70%, 80%, 90%, 100%, 2-fold, 5-fold, 10-fold, 100-fold, 1000-fold or more compared to when the antigen or nucleic acid encoding the antigen is administered alone. One skilled in the art is familiar with how to evaluate the level of antibody titer e.g., by ELISA.

In some embodiments, the adjuvantation system prolongs the effect of a vaccine (e.g., by at least 20%) in the subject compared to when the antigen or nucleic acid encoding the antigen is administered alone. For example, the adjuvantation system may prolong the effect of a vaccine by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more in the subject compared to when the antigen or nucleic acid encoding the antigen is administered alone. In some embodiments, the adjuvantation system prolongs the effect of a vaccine by 20%, 30%, 40%, 50%, 60%, 70%, 80%, 90%, 100%, 2-fold, 5-fold, 10-fold, 100-fold, 1000-fold or more compared to when the antigen or nucleic acid encoding the antigen is administered alone.

In some embodiments, the immunogenic compositions (e.g., vaccine compositions) described herein induce an immune response to a Beta coronavirus antigen (e.g., an antigen from MERS-CoV, SARS-CoV-1, or SARS-CoV-2) or to a Beta coronavirus (e.g., MERS-CoV, SARS-CoV-1, or SARS-CoV-2). In some embodiments, the immunogenic compositions (e.g., vaccine compositions) described herein induce an immune response against MERS-CoV, SARS-CoV-1, and/or SARS-CoV-2. Heterologous immunity is contemplated herein. Heterologous immunity refers to a phenomenon by which antigen-specific responses that were generated against one pathogen are reactivated in response to a second pathogen. For example, the immunogenic composition (e.g., vaccine composition) may comprise a SARS-CoV-1 antigen (e.g., a SARS-CoV-1 protein or polypeptide, or a nucleic acid encoding a SARS-CoV-1 protein or polypeptide) and induce an immune response to both SARS-CoV-1 and SARS-CoV-2. Similarly, the immunogenic composition (e.g., vaccine composition) may comprise a SARS-CoV-2 antigen (e.g., a SARS-CoV-2 protein or polypeptide, or a nucleic acid encoding a SARS-CoV-2 protein or polypeptide) and induce an immune response to both SARS-CoV-1 and SARS-CoV-2.

Heterotypic immunity is also contemplated herein. Heterotypic immunity refers to a phenomenon by which antigen-specific responses that were generated against a serotype or strain are reactivated in response to a different serotype or strain. For example, the immunogenic composition (e.g., vaccine composition) may comprise a SARS-CoV-2 WA1 antigen (e.g., a SARS-CoV-2 WA1 protein or polypeptide, or a nucleic acid encoding a SARS-CoV-2 WA1 protein or polypeptide) and induce an immune response to both SARS-CoV-2 WA1 and SARS-CoV-2 B.1.1.529. Similarly, the immunogenic composition (e.g., vaccine composition) may comprise a SARS-CoV-2 B.1.1.529 antigen (e.g., a SARS-CoV-2 B.1.1.529 protein or polypeptide, or a nucleic acid encoding a SARS-CoV-2 B.1.1.529 protein or polypeptide) and induce an immune response to both SARS-CoV-2 WA1 and SARS-CoV-2 B.1.1.529. Heterologous and heterotypic immunity can arise as a result of shared antigen structure and/or function (e.g., antigens having similar or identical structure and/or function among different pathogens, strains, serotypes), leading to the production of cross-reactive antigen-specific antibodies. Thus, in some embodiments, the immunogenic compositions (e.g., vaccine compositions) described herein enhance the production of cross-reactive antigen-specific antibodies, compared to when the antigen or nucleic acid encoding the antigen is administered alone. In some embodiments, the immunogenic compositions (e.g., vaccine composition) described herein enhance the production of cross-reactive antigen-specific antibodies by at least 2-fold, at least 5-fold, at least 10-fold, at least 100-fold, or at least 100-fold or more, compared to when the antigen or nucleic acid encoding the antigen is administered alone. In some embodiments, the cross-reactive antigen-specific antibodies target variants of the antigen.

### EXAMPLES

### Example 1: Separate Injections of Comirnaty and Alum-Mannan Increase the Titers of Neutralizing Antibodies Directed Against SARS-CoV-2 WA1, but not B.1.1.529

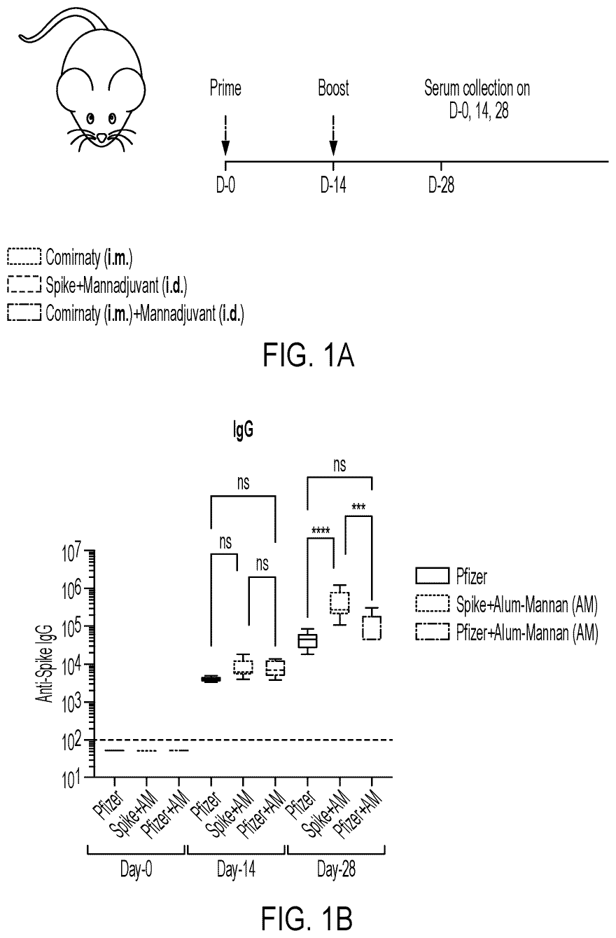

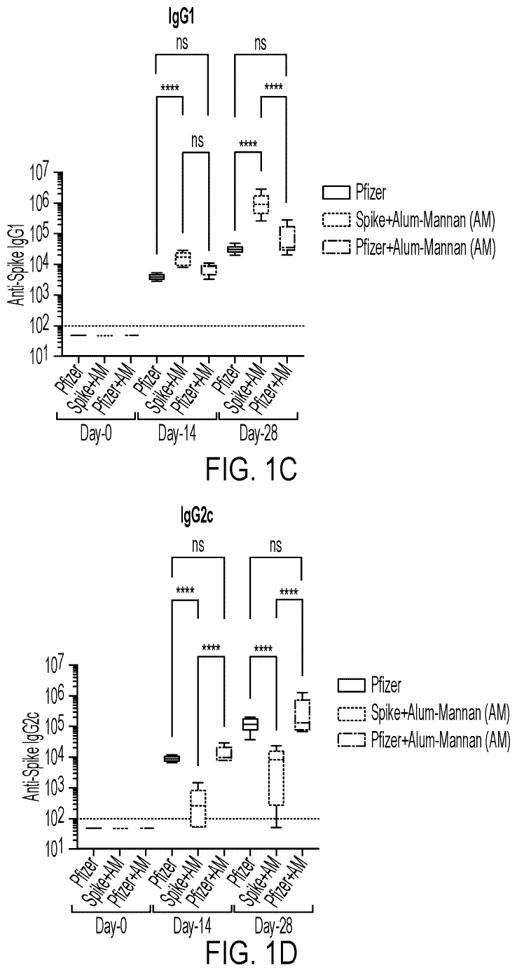

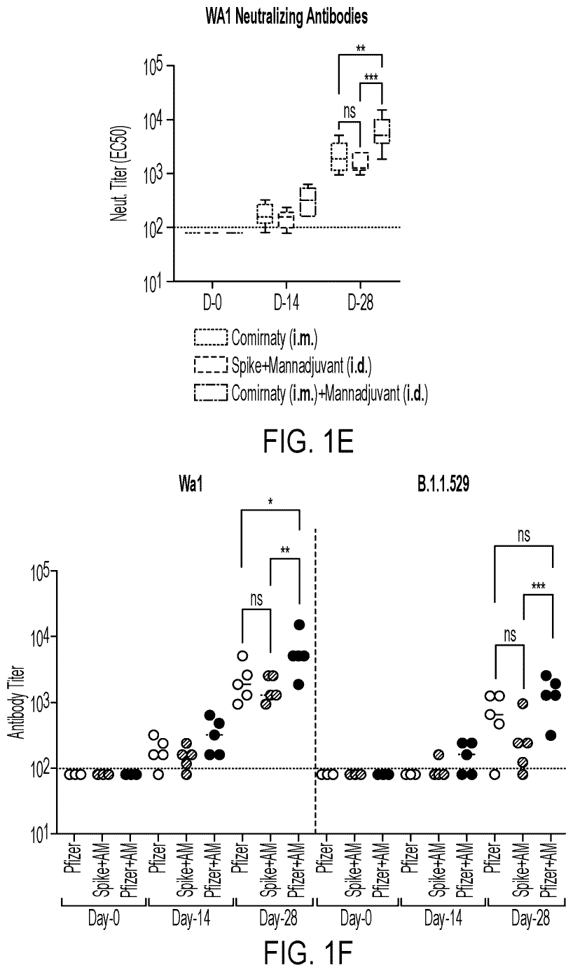

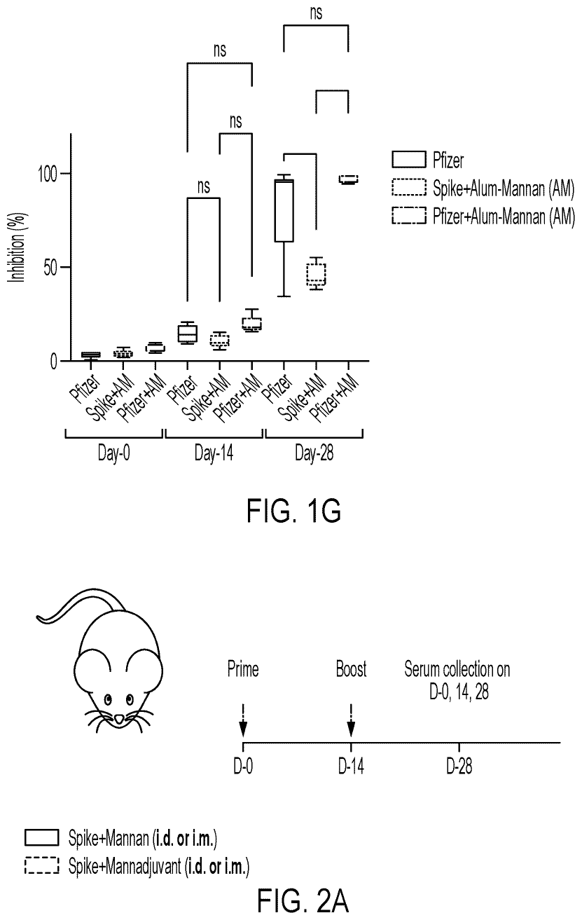

This Example tested whether alum-mannan potentiates the activity of Comirnaty (BNT162b2 mRNA, Pfizer-BioNTech). Alum-mannan was formulated with a weight ratio of alum:mannan=1:5. Comirnaty was injected on day 0 (prime) and day 14 (boost) intramuscularly with or without intradermal injection of alum-mannan and compared with intramuscular injection of the pre-fusion stabilized SARS-CoV-2 WA1 Spike trimer (Spike) with intradermal injection of alum-mannan (FIG. 1A). The levels of anti-Spike antibody and their neutralization activity was determined 14 days post-prime (day 14) or 14 days post-boost (day 28). No significant change in the total amount of anti-Spike antibody was detected when Comirnaty was injected with or without alum-mannan (FIG. 1B). However, a significant increase in the serum levels of anti-Spike protein antibodies was observed 28 days after the immunization with alum-mannan and the Spike protein, compared to both mice immunized with Comirnaty alone or with alum-mannan. While a similar trend was maintained for the anti-Spike protein IgG1 (FIG. 1C), anti-Spike protein IgG2c was more efficiently induced when mice were immunized with Comirnaty, regardless of the presence or absence of alum-mannan (FIG. 1D).

Although antibody levels may serve as correlates of protection against SARS-CoV-2, it is neutralizing antibodies that provide a more accurate prediction of immune protection. The sera of mice that were immunized with Comirnaty and alum-mannan showed an increased neutralization activity against SARS-CoV-2 WA1 compared to Comirnaty alone or Spike protein and alum-mannan (FIG. 1E). However, the neutralization activity against SARS-CoV-2 B.1.1.529 of sera derived from mice immunized Comirnaty and alum-mannan was not significantly increased compared to mice that received Comirnaty alone (FIG. 1F). Finally, a surrogate virus neutralization test showed no significant difference in the activity of Comirnaty with or without alum-mannan (FIG. 1G). Overall, these data demonstrate that the draining of alum-mannan to the same draining lymph node targeted by Comirnaty increases the induction of neutralizing antibodies against the Spike protein encoded by Comirnaty.

### Example 2: Alum-Mannan Formulation Maintains its Adjuvant Activity when Injected Intramuscularly

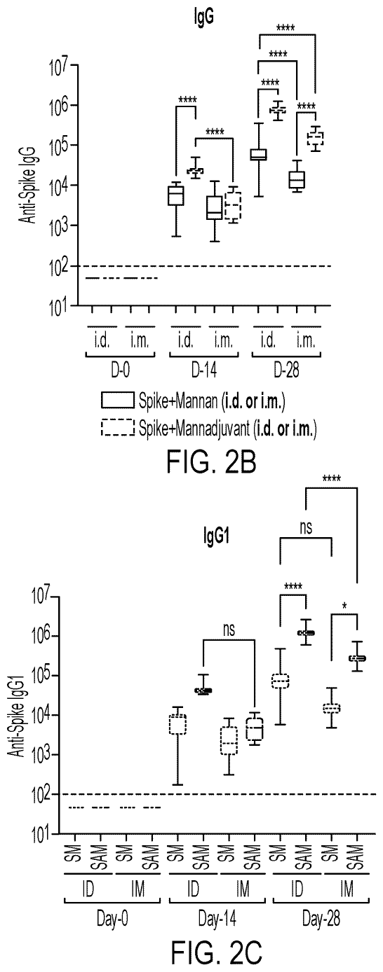

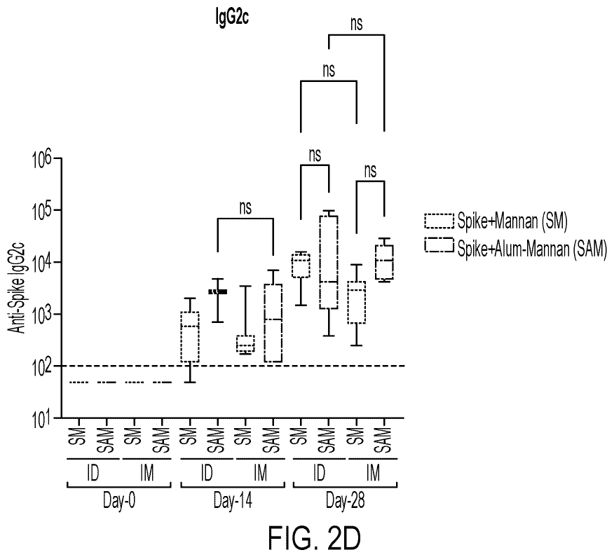

It was previously demonstrated that in order to exert its adjuvant activity, alum-mannan injected in the epidermis is directly drained to the draining lymph node. However, it was completely unknown whether another route of immunization, such as intramuscular injection, would maintain the adjuvant activity of alum-mannan. To test the possibility to inject alum-mannan intramuscularly, a study was performed to compare intramuscular and intradermal injection of pre-fusion stabilized SARS-CoV-2 WA1 Spike trimer (Spike) with mannan or alum-mannan. Mice were immunized with a prime-boost schedule with alum-mannan or the mannan and a Spike protein intradermally or intramuscularly (FIG. 2A). Anti-Spike protein IgG levels were measured at 14- and 28-days post-immunization. The results of this study revealed the capacity of alum-mannan to improve the levels of antibodies, compared to the mannan alone, was maintained when administered intramuscularly (FIGS. 2B-2D). The data also showed that the antibody response was significantly higher in when administered intradermally, as compared to when administered intramuscularly, suggesting differences in the draining to the draining lymph node or in the nature of the cells in the tissue that can respond to glycan-containing adjuvant formulations. These data demonstrate the possibility to inject Comirnaty and alum-mannan simultaneously in the muscle and evaluate their possible synergy.

### Example 3: Stability of a Fixed-Dose Combination (FDC) Vaccine

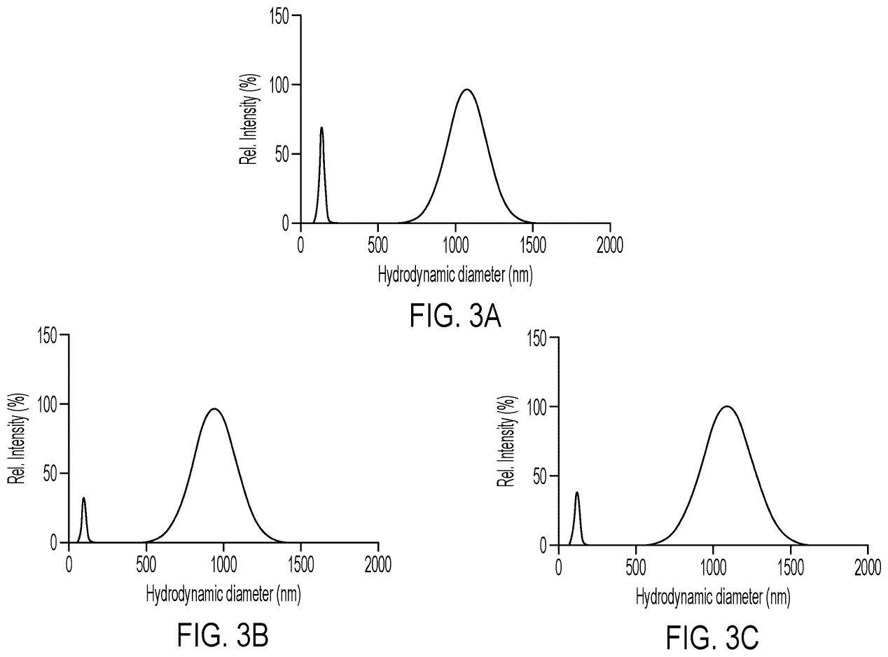

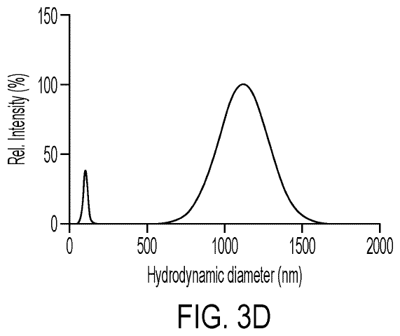

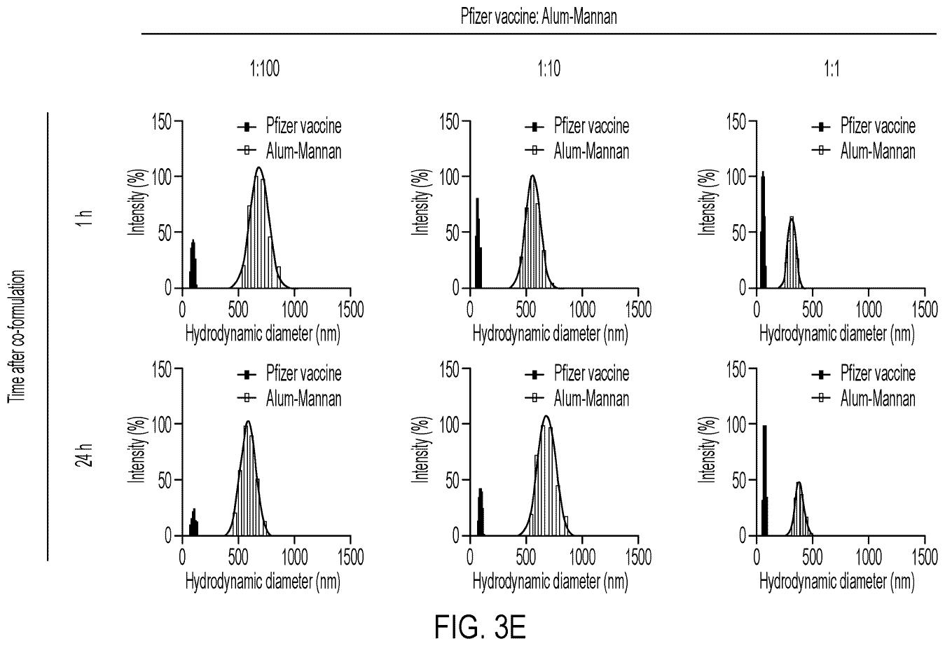

This Example evaluated whether alum-mannan and lipid nanoparticles (LNPs) can be admixed without disrupting either component. First, dynamic light scattering was performed on lipid nanoparticles admixed at different ratios with mannan-alum. First, 10 μg/ml alum was mixed for 30 minutes with increasing concentrations of mannan to obtain alum-mannan mixtures with weight ratios of 1:1 (FIG. 3A), 1:2 (FIG. 3B), 1:5 (FIG. 3C), and 1:10 (FIG. 3D), followed by a 1 hour incubation with 10 μg/ml Comirnaty liposomal nanoparticles. Clear separation of peaks for all alum:mannan weight ratios were observed by dynamic light scattering, and no alteration in the size of either the LNPs or the alum-mannan was detected.

Next, the stability of Comirnaty lipid nanoparticles in the presence of excess alum-mannan was evaluated. Comirnaty (1 μg/ml) was incubated with increasing concentrations of alum-mannan formulated at a weight ratio of 1:100 (alum:mannan) and evaluated using dynamic light scattering. Clear separation of peaks for all Comirnaty:alum-mannan weight ratios were observed both 1 hour and 24 hours post-incubation, demonstrating the lipid shell of Comirnaty remains stable when co-formulated with alum-mannan and that Comirnaty does not physically interact with alum-mannan (FIG. 3E).

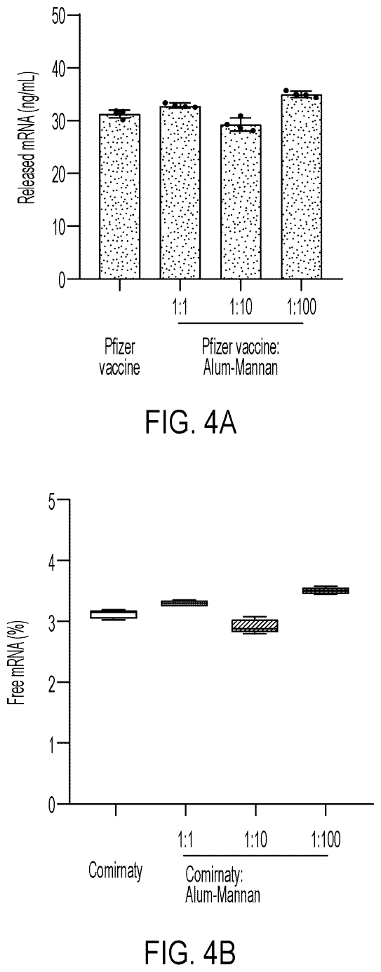

These results were confirmed in a separate experiment, in which the total mRNA in solution was quantified as a proxy of the lipid shell dissolution of Comirnaty and resultant premature mRNA release. Comirnaty (1 μg mRNA) and alum-mannan (1:100 weight ratio of alum:mannan) were admixed and incubated for 24 hours at 4° C., and the free mRNA in solution was determined using Quant-it™ RiboGreen RNA Assay it (Thermo Fisher Scientific). Released mRNA was quantified (FIG. 4A) and evaluated as a percentage of total mRNA (FIG. 4B), showing that all FDCs exhibited a minimal degree of mRNA release (~3-4%) even when the alum-mannan complex was in 100-fold excess. Together, these results indicate that Comirnaty is physically and chemically stable in the presence of excess alum-mannan complex. The data demonstrates that alum-mannan maintains its adjuvant activity when injected intramuscularly, but also that alum-mannan and Comirnaty can be admixed without altering the physical and chemical properties of both particles.

### Example 4: FDCs Potentiate the Induction of Anti-Spike IgGs, Increases their Neutralization Capacity, and can be Efficiently Re-Boosted with a Third Dose

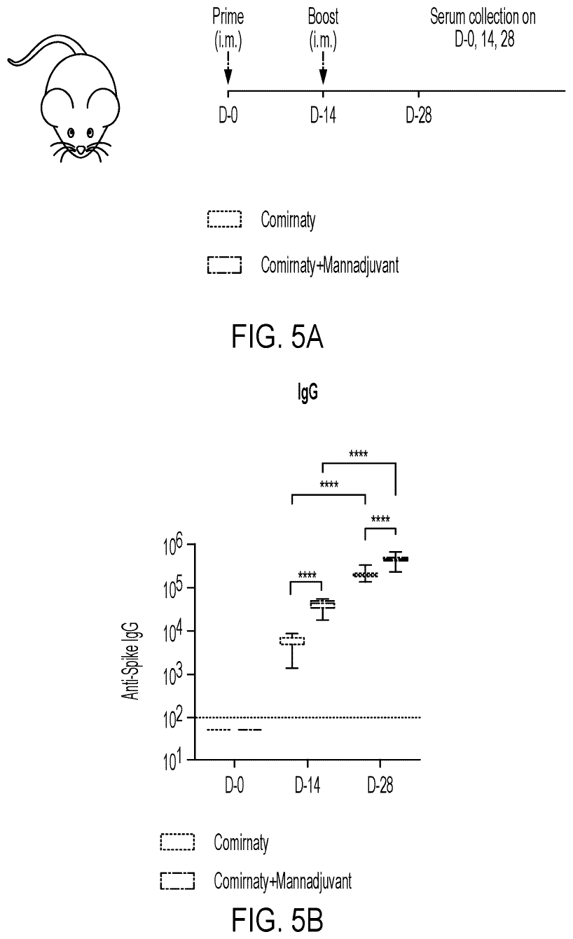

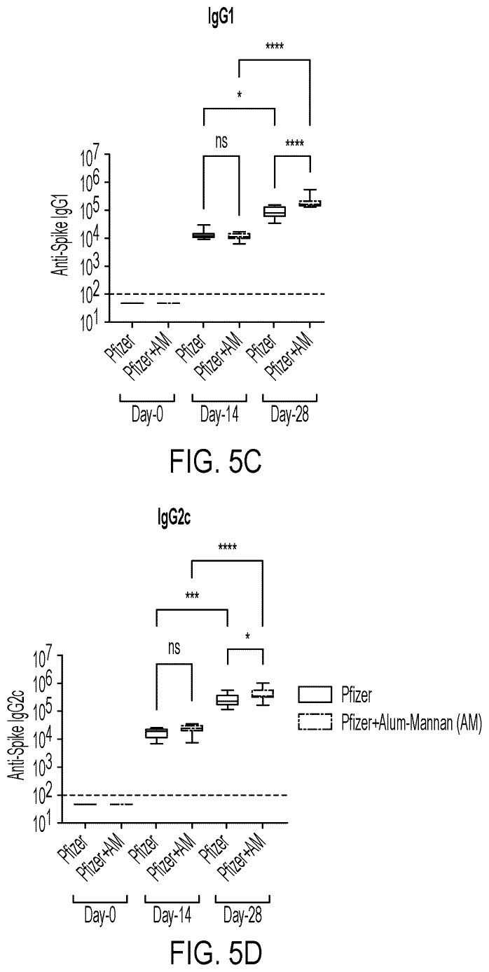

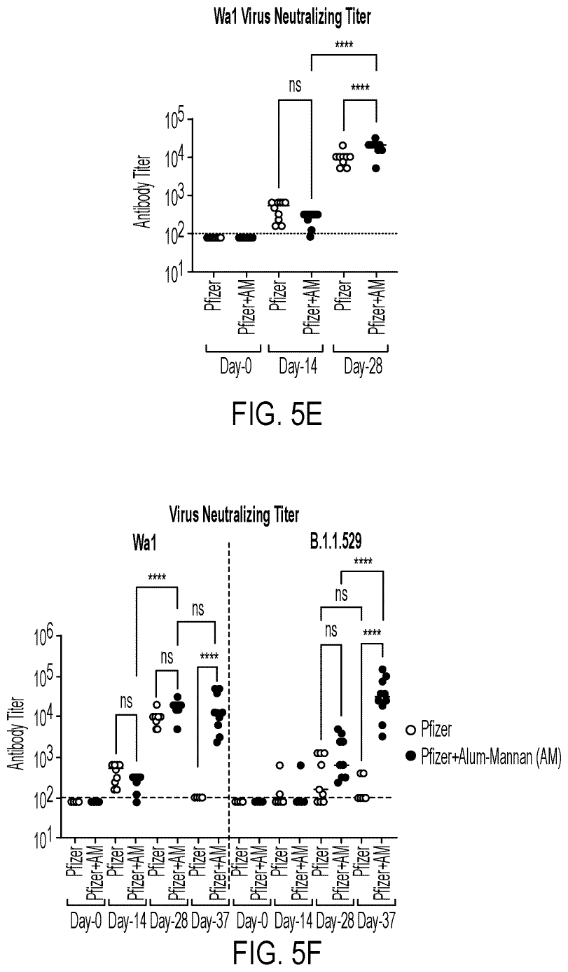

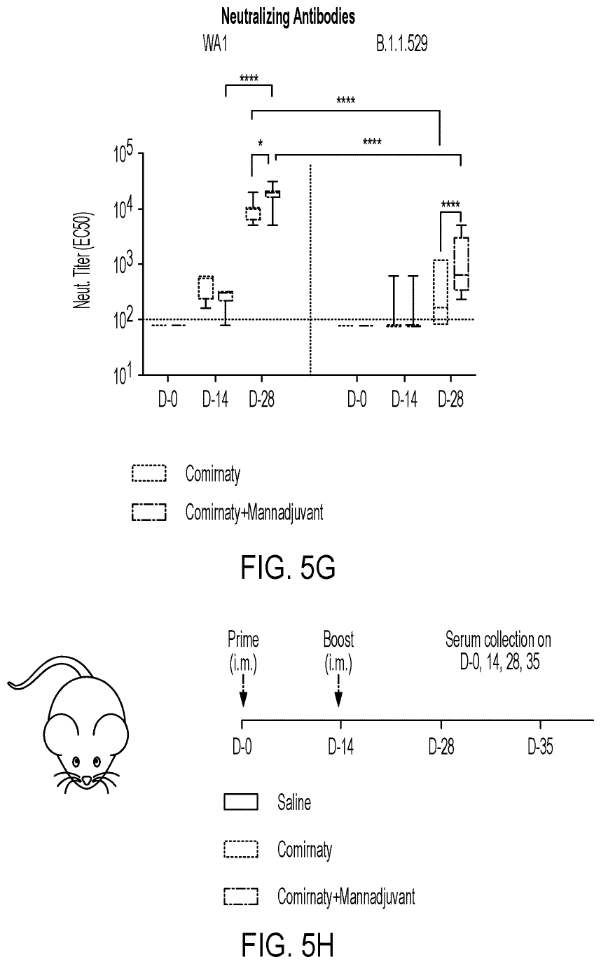

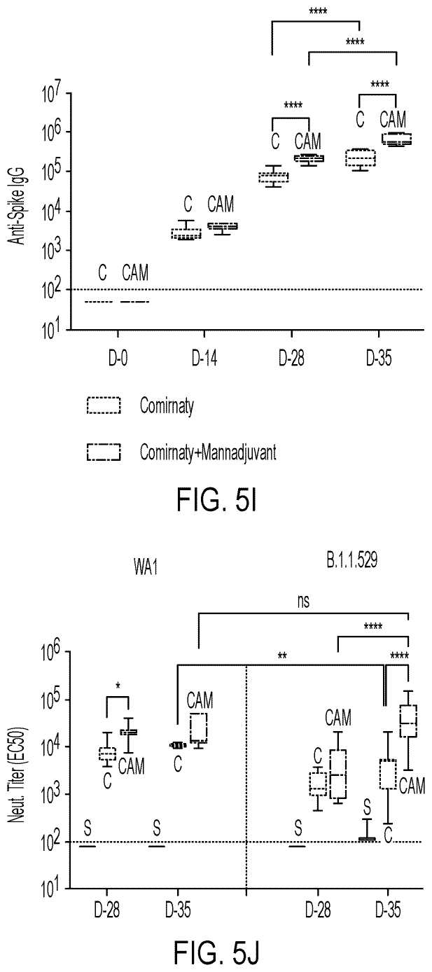

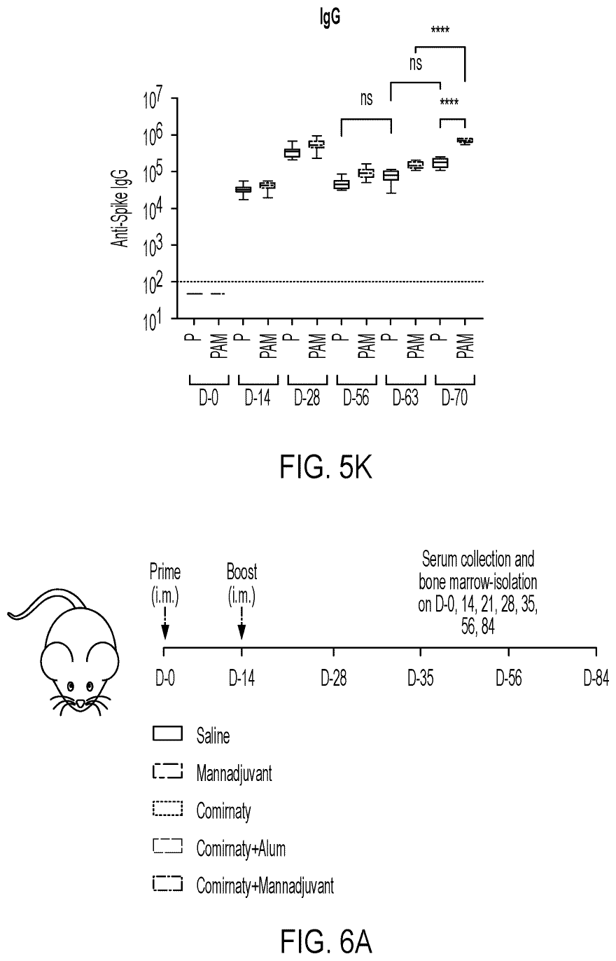

To evaluate whether the FDC vaccines ameliorated the response elicited by Comirnaty, mice were prime-boosted intramuscularly with Comirnaty or the FDC vaccine (Comirnaty admixed with alum-mannan) (FIG. 5A). Serum levels of anti-Spike IgGs were significantly increased when mice were immunized with the FDC vaccine compared to mice immunized with Comirnaty alone (FIG. 5B). Anti-Spike protein IgG1 and IgG2c followed a similar trend (FIGS. 5C-5D). Of note, 28-days after immunization, the serum of mice that received the FDC vaccine showed a significant increase in the titers of neutralizing antibodies directed against SARS-CoV-2 WA1 as compared to mice that received Comirnaty alone (FIG. 5E).

Since SARS-CoV-2 is a rapidly evolving virus, neutralizing antibodies directed against a highly divergent variant of interest was also evaluated. In keeping with the reduced effectiveness of Comirnaty against SARS-CoV-2 WA1, mice immunized with Comirnaty showed decreased neutralizing antibodies against SARS-CoV-2-B.1.1.529 compared to neutralizing antibodies against SARS-CoV-2-WA1 (FIGS. 5F-5G). Although this was also true for mice immunized with the FDC vaccine, the levels of neutralizing antibodies in these mice were significantly increased compared to mice that were immunized with Comirnaty alone. These data suggest that the immunization with Comirnaty admixed with alum-mannan can foster the induction of cross-reactive neutralizing antibodies.

Thus, the observation was prolonged to 5 weeks post-initial immunization (FIG. 5H). At this time point, the levels of anti-Spike protein IgG levels were significantly augmented compared to week 4 (day 28) under all experimental conditions (FIG. 5I). Notably, 5 weeks after the initial immunization, mice that received the FDC vaccine, but not mice that received Comirnaty alone, presented neutralizing antibodies against SARS-CoV-2 B.1.1.529 that were as high as the neutralizing antibodies directed against SARS-CoV-2 WA1 (FIG. 5J).

Finally, it was found that the anti-Spike IgG levels decreased between day 28 and day 56 post-priming for both formulations (Comirnaty and the FDC vaccine). Notably, a third dose of the FDC vaccine administered at day 56 induced a significant increase in anti-Spike IgG levels (FIG. 5K). However, a similar response was not seen in mice administered a third dose of Comirnaty at day 56, suggesting that the FDC vaccine can favor better recall responses.

### Example 5: The Glycan Component of Alum-Mannan is Necessary to Extend the Duration of Antibody Production Elicited by Comirnaty

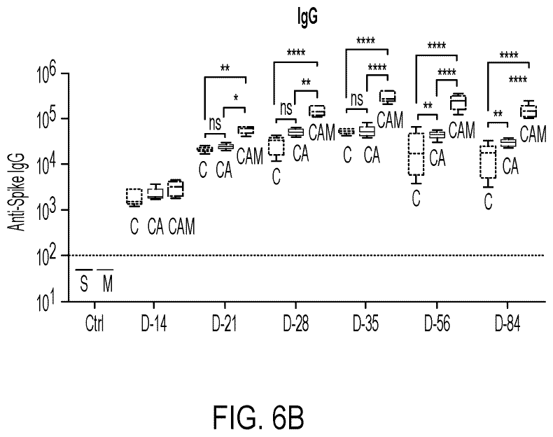

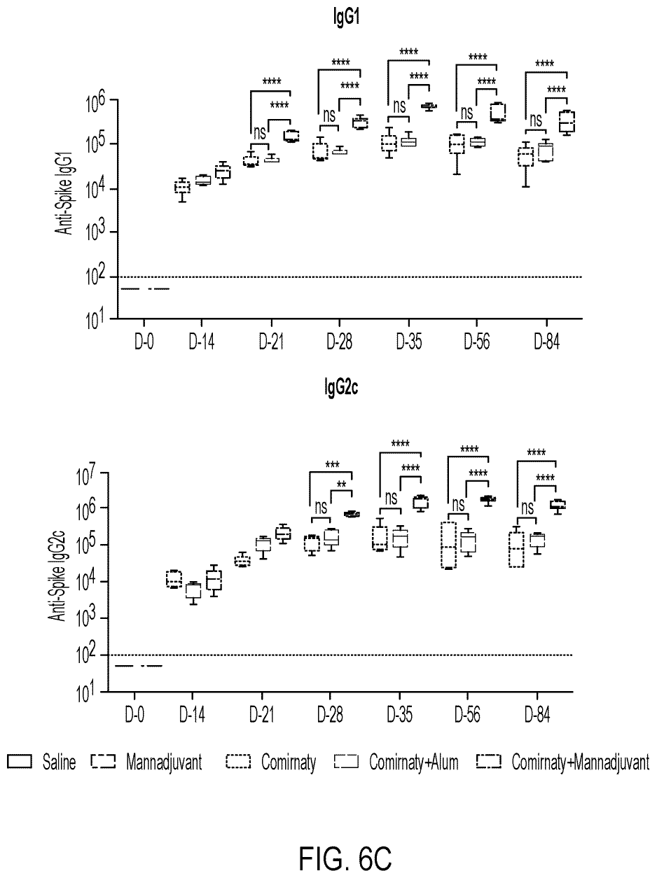

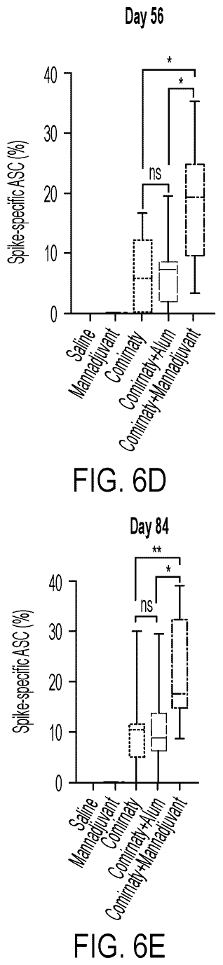

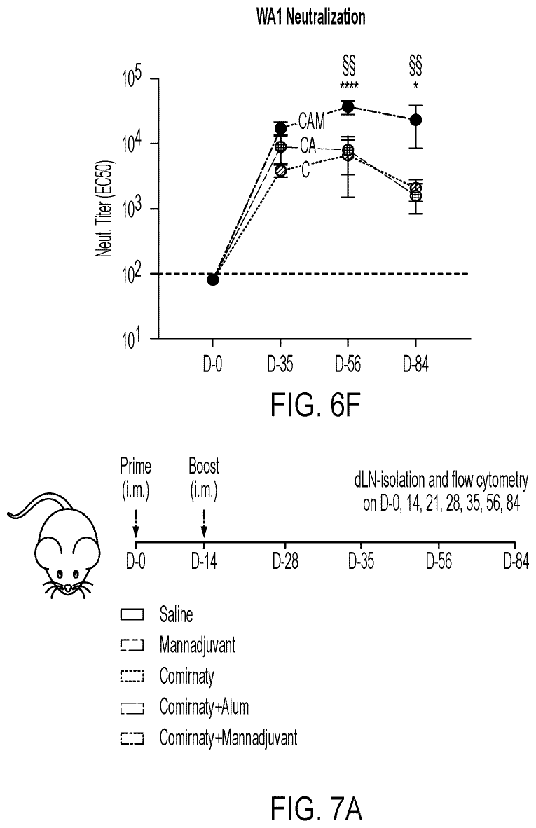

Since it was shown that 5 weeks after initial immunization the anti-Spike protein antibody levels in mice that received the FDC vaccine were still increasing, it was hypothesized that alum-mannan extends the duration of the response elicited by Comirnaty. Thus, the analyses were prolonged to days 56- and 85-post-initial immunization (FIG. 6A). Anti-Spike protein antibody levels remained significantly higher for the entire duration of the experiment in mice immunized with the FDC vaccine, compared to mice that were immunized only with Comirnaty (FIGS. 6B-6C).

Additionally, to determine if the prolonged duration of the antibody response elicited by the FDC vaccine can be attributed to alum, the mice were also immunized with alum and Comirnaty (FIG. 6A). The results showed that the anti-Spike protein total IgG, as well as IgG1 and IgG2c, levels were significantly decreased in these mice, compared to mice immunized with the FDC vaccine (FIGS. 6D-6E). These data demonstrate that the mannan contained in the FDC vaccine is required to extend the activity of Comirnaty.

Next, the persistence of anti-Spike protein IgG in mice that received the FDC vaccine was evaluated to determine whether this phenomenon was for reflected in bone marrow resident long-lived plasma cells. The number of SARS-CoV-1 WA1 Spike protein-specific plasma cells present in the bone marrow of mice immunized with the FDC vaccine, but not Comirnaty with alum, was significantly increased in both 56-days and 85-days post-immunization (FIGS. 6C-6D). Further, the neutralizing activity against SARS-CoV-2 WA1 of antibodies present in the serum of mice immunized with the FDC vaccine, but not Comirnaty with alum, was significantly extended compared to mice that received Comirnaty alone (FIG. 6F).

### Example 6: Alum-Mannan Expands Germinal Center Responses and Allows the Generation of B Cells that Recognize Distinct Variants of Concern (VOCs)

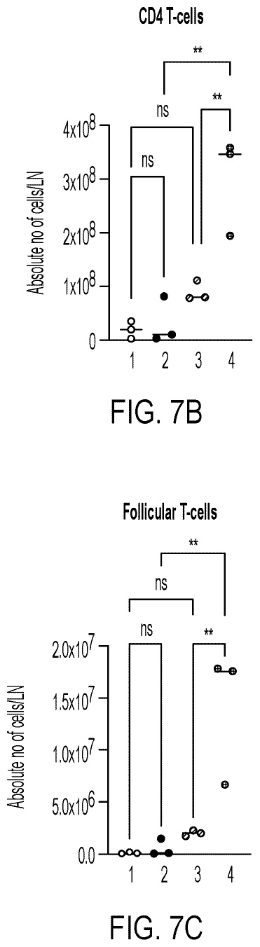

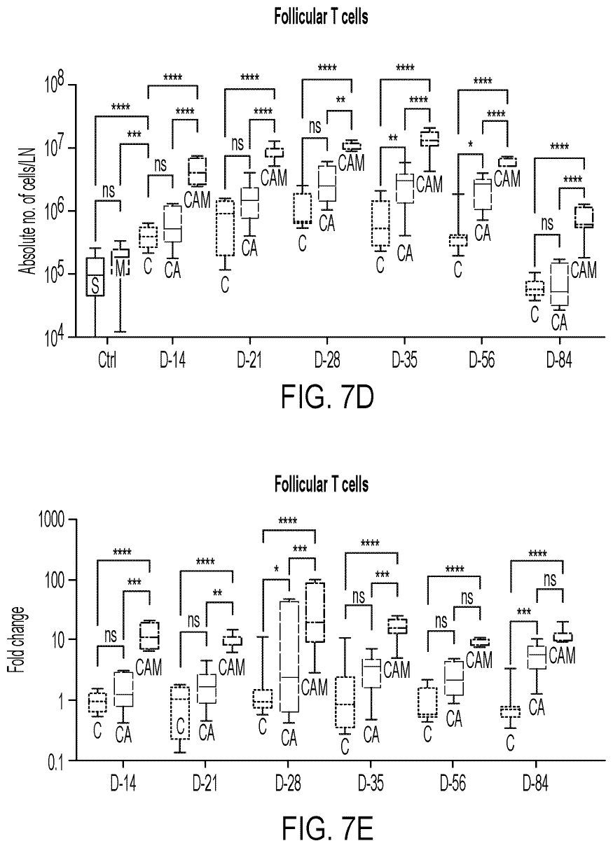

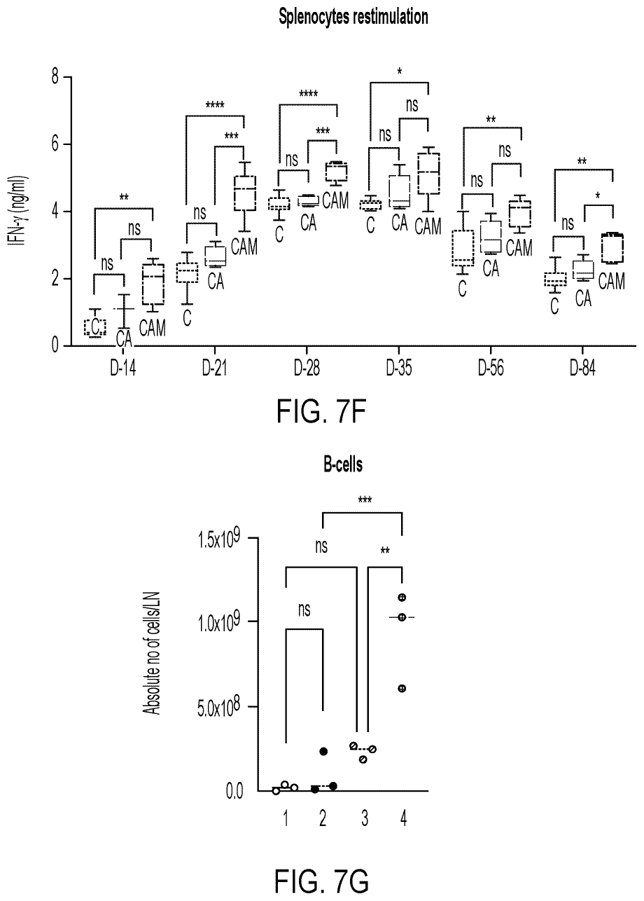

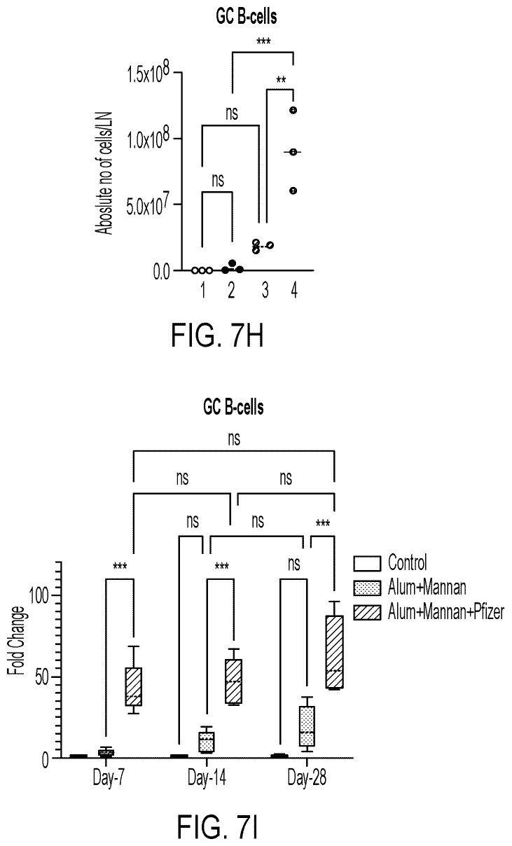

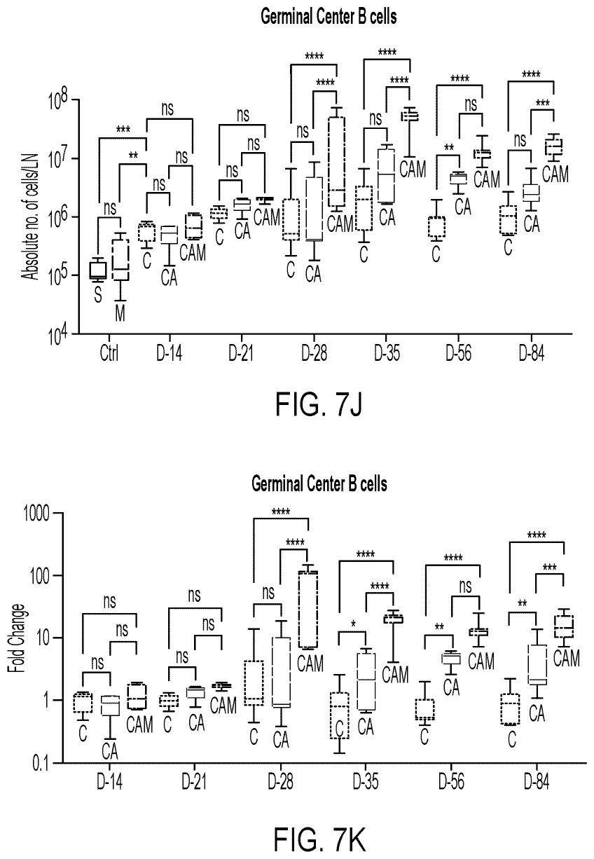

This Example demonstrates the mechanism by which alum-mannan changes the capacity of Comirnaty to induce antibodies. Follicular T cells and formation of germinal centers (GCs) regulate the generation of the antibody response. Studies in humans demonstrated that Comirnaty induces a strong response both in terms of the generation of follicular T cells, as well as of GCs. Thus, the generation of follicular T cells and GCs in mice were assessed in mice (FIG. 7A). The results confirmed that Comirnaty induces an efficient expansion of T-follicular cells. Additionally, the data showed that mice that also received Comirnaty with alum-mannan, but not Comirnaty with alum, presented a significant increase in the number of T follicular cells (FIGS. 7B-D). The increase in the generation of follicular T cells started 14 days post-prime and persisted for the entire duration of the experiment. Naïve mice or mice injected with alum-mannan alone were utilized as controls. When compared with the number of follicular T cells elicited by Comirnaty, immunization with Comirnaty and alum-mannan, but not Comirnaty and alum, induced a long-fold increase (FIG. 7E). The potentiation of T cell responses by alum-mannan was not limited to follicular resident cells. Splenic T cell restimulated with the SARS-CoV-2 WA1 Spike protein peptides responded by producing increased amounts of interferon (IFN)-γ (FIG. 7F).

Next, the expansion of GC B cells in the draining lymph node of mice was evaluated in naïve mice, or mice administered Comirnaty (BNT162b2 mRNA, Pfizer-BioNTech), Comirnaty with alum, or Comirnaty with alum-mannan. While 14 days after prime, there were no major differences in the number of germinal center B cells, differences became evident starting 4 weeks after initial immunization (FIGS. 7J-7K). The number of GC B cells in mice immunized with Comirnaty and alum-mannan was significantly increased and remained more stable over time, compared to mice that received Comirnaty alone or with alum.

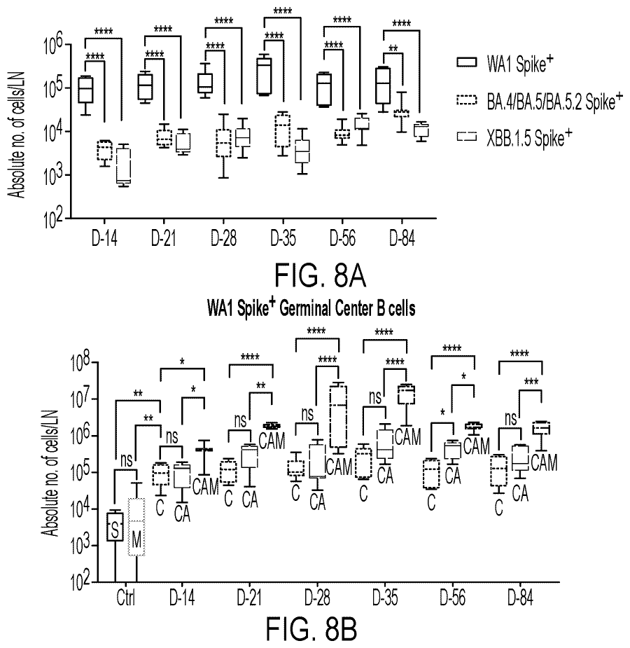

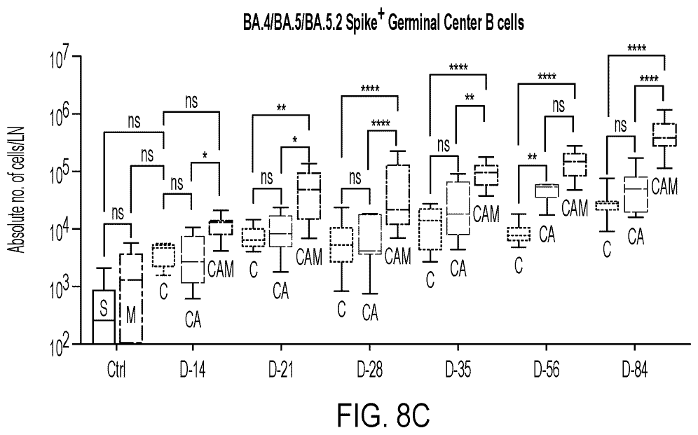

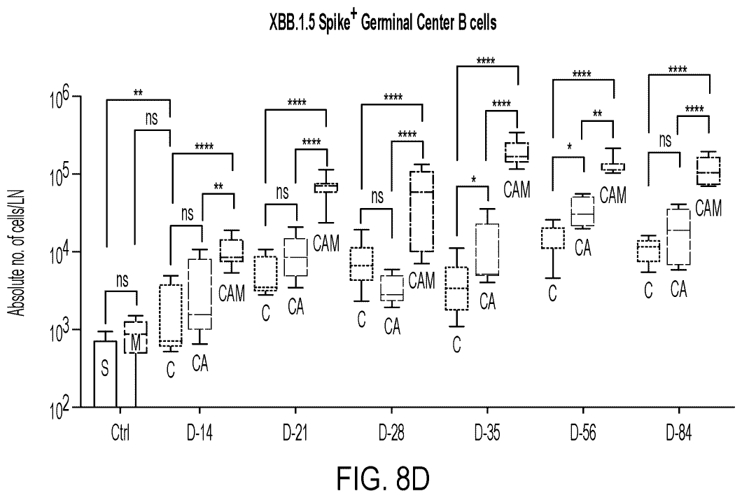

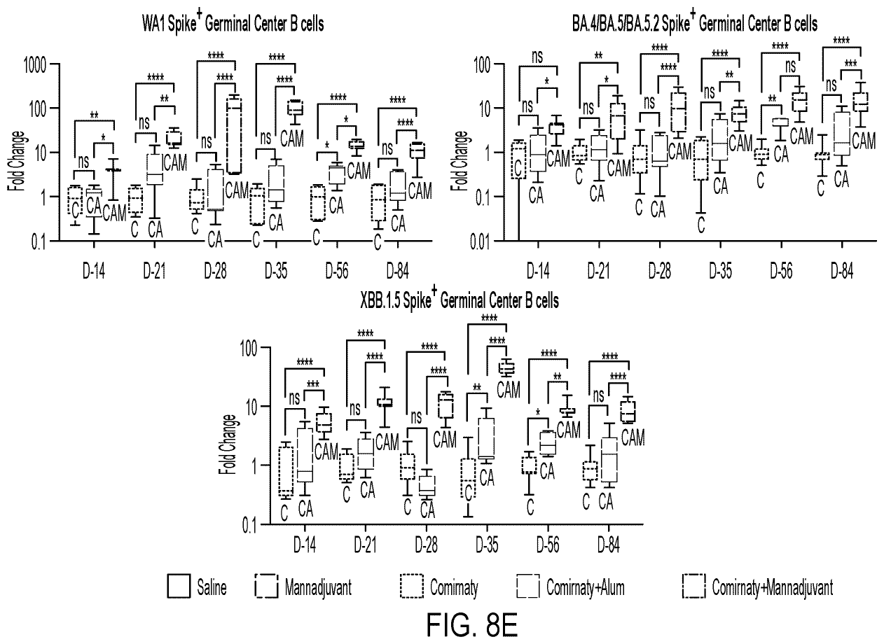

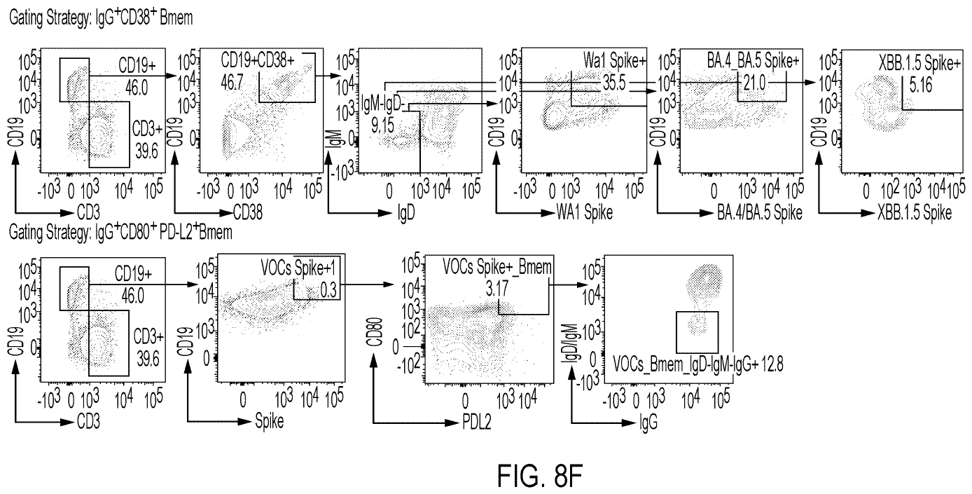

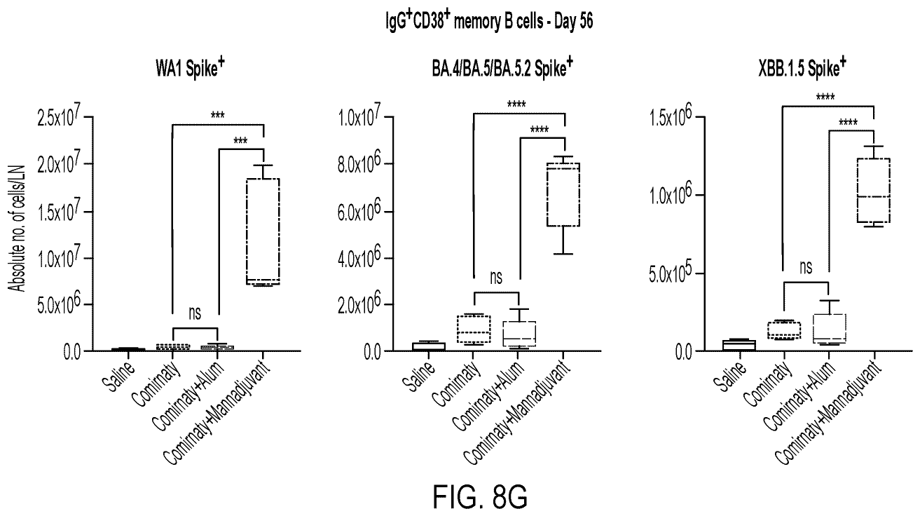

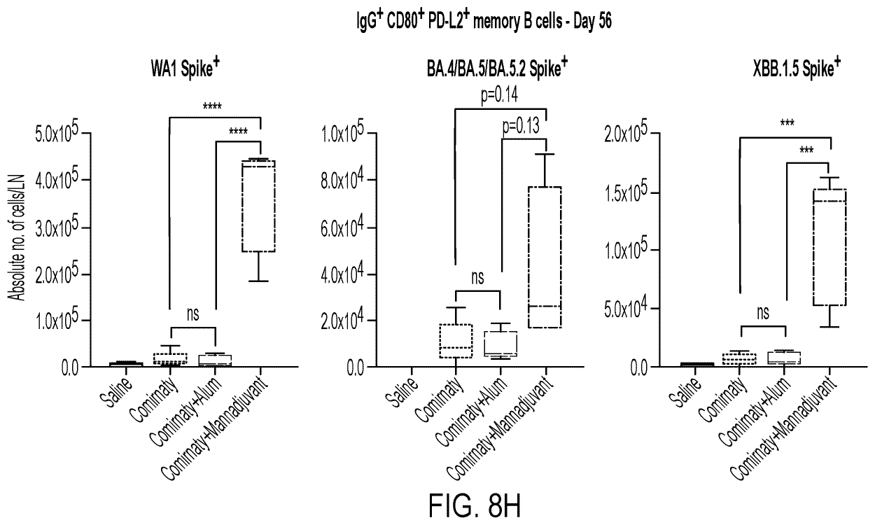

Based on the persistence of follicular T cells and GC B cells, and the capacity of alum-mannan to induce neutralizing antibodies against SARS-CoV-2 B.1.1.529, the ability for GC B cells to acquire the capacity to recognize the Spike protein of Omicron sublineages (BA.4, BA.5, and BA.5.2, or XBB.1.5) was evaluated. These variants of concern, due to extensive mutations in the Spike protein of SARS-CoV-2 B.1.1.529, acquired increased evasion properties. In keeping with the limited capacity of Comirnaty to protect against these Omicron sublineages, the numbers of B cells that recognized the Spike protein of SARS-CoV-2 BA.4, BA.5, or XBB.1.5 were significantly lower compared to the number of B cells that recognized the Spike protein of SARS-CoV-2 WA1 (FIG. 8A). Notably, administration with Comirnaty and alum-mannan, but not Comirnaty with alum, significantly increased the induction of B cells that expressed a B cell receptor capable of recognizing the Spike protein of SARS-CoV-2 BA.4/BA.5, as well as SARS-CoV-2 XBB.1.5 (FIGS. 8B-8E).

Finally, the affect on B memory cell formation was evaluated. B memory cells were identified based on their expression of IgG and CD38 (IgG⁺CD38⁺B mem), or as IgG⁺CD80⁺PD-L2⁺ cells (FIG. 8F). The absolute number of B memory cells that recognized all SARS-CoV-2 strains tested were significantly increased 56-days after immunization with Comirnaty and alum-mannan, compared to Comirnaty alone or with alum (FIGS. 8G-8H). Overall, these data demonstrate that adjuvantation of Comirnaty with alum-mannan expands follicular T cells, GC B cells, and B memory cells against multiple Omicron sublineages.

### Example 7: Glycan Adjuvantation of Comirnaty Expands the Formation of Antibodies that Neutralize Distinct Omicron Sublineages

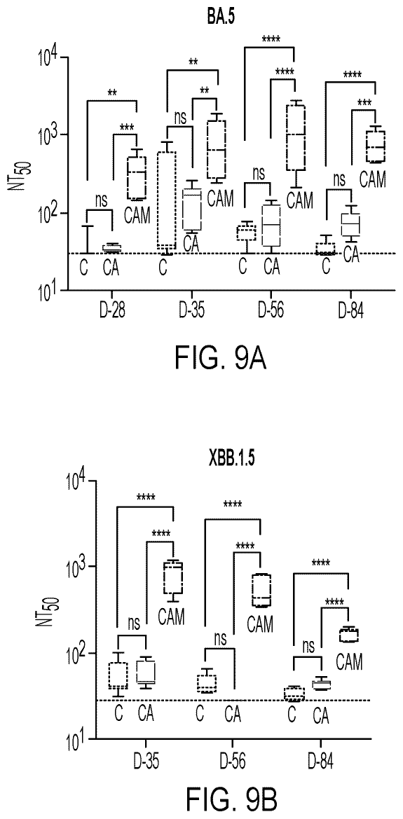

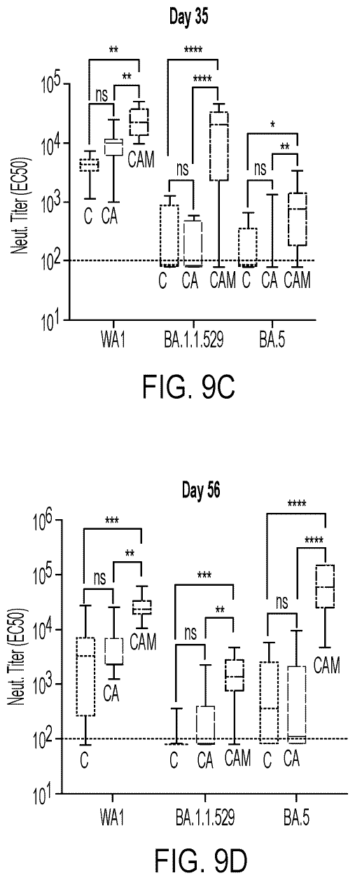

The formation of B cells that express B cell receptors recognizing the Spike protein of distinct Omicron sublineages opens the possibility that mice immunized with Comirnaty and alum-mannan can also develop neutralizing antibodies against these variants of concern. The capacity of the serum of immunized mice to neutralize pseudoviruses that express the Spike protein of SARS-CoV-2 BA.5 or XBB.1.5 was evaluated. Both pseudoviruses were neutralized with the serum of mice immunized with Comirnaty and alum-mannan, but not Comirnaty alone or with alum (FIGS. 9A-9B).

The neutralization was next performed against SARS-CoV-2 B.1.1.529 and BA.5, while SARS-CoV-2 WA1 was used as a control. In line with the previous results, the serum of mice immunized with Comirnaty and alum-mannan, but not Comirnaty alone or with alum, efficiently neutralized all the variants of concern tested 35- and 56-days after the initial immunization (FIGS. 9C-9D). Overall, these results show that alum-mannan expands the capacity of Comirnaty to drive neutralizing antibodies against Omicron sublineages characterized by a high escape capacity.

### Example 8: Glycan Adjuvantation of Comirnaty Alters the Inflammatory Programs in the Draining Lymph Node Early Upon Immunization

The data demonstrated that alum-mannan, but not alum, changes the responses elicited by Comirnaty and allows the potentiation of T and B cell responses in the draining lymph node. To get a better understanding of the programs that are regulated and/or amplified by alum-mannan in mice immunized with Comirnaty, bulk RNA sequencing (RNAseq) was performed on the draining lymph node of mice at 1, 3, and 7 days post-priming with Comirnaty (BNT162b2 mRNA, Pfizer-BioNTech), Comirnaty and alum, Comirnaty and alum-mannan, or saline-injected controls.

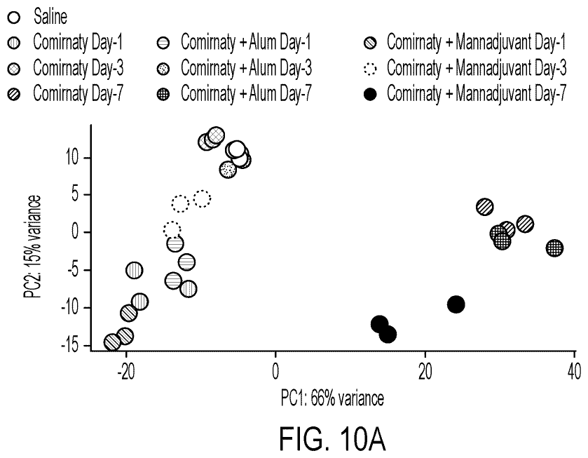

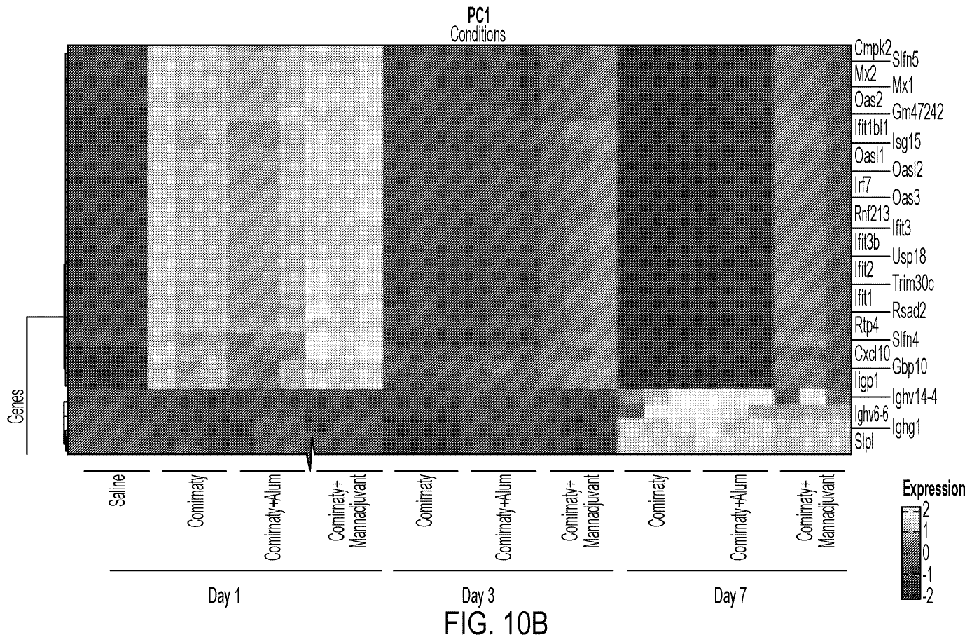

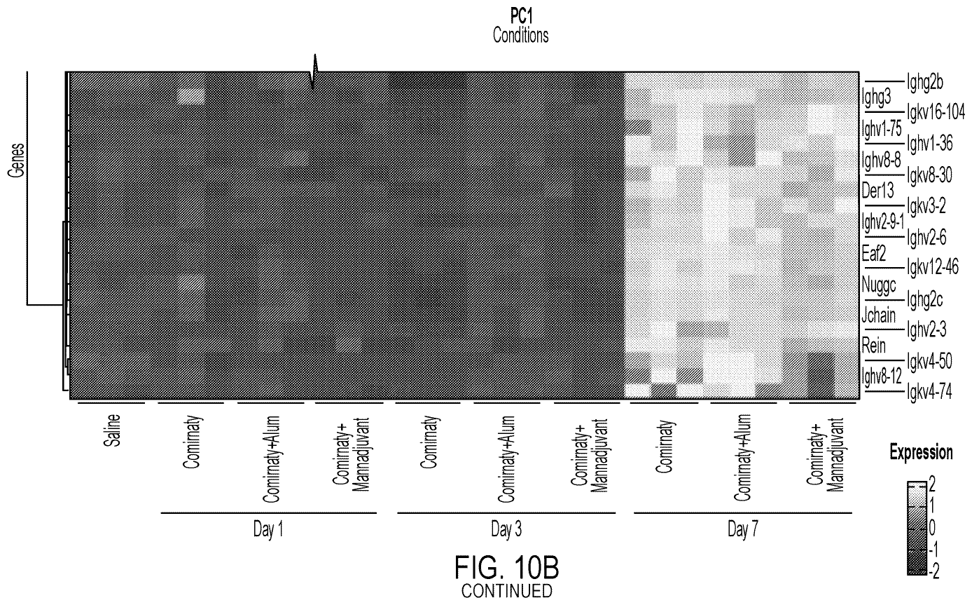

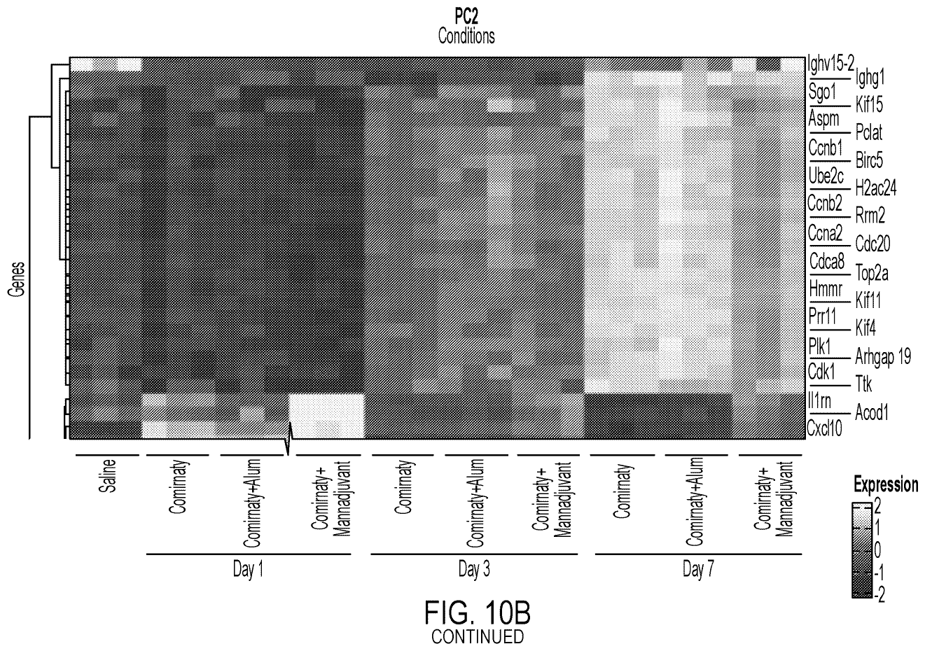

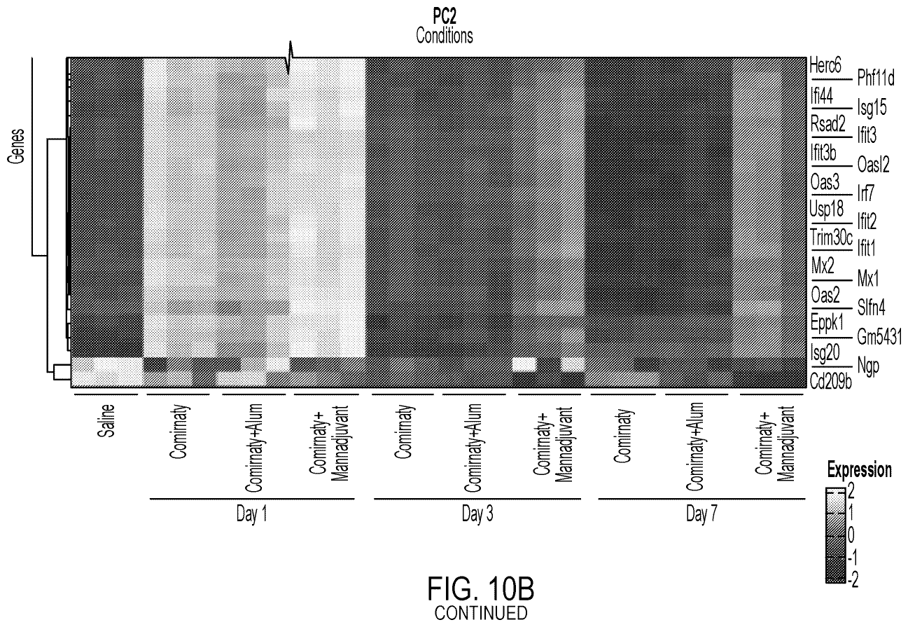

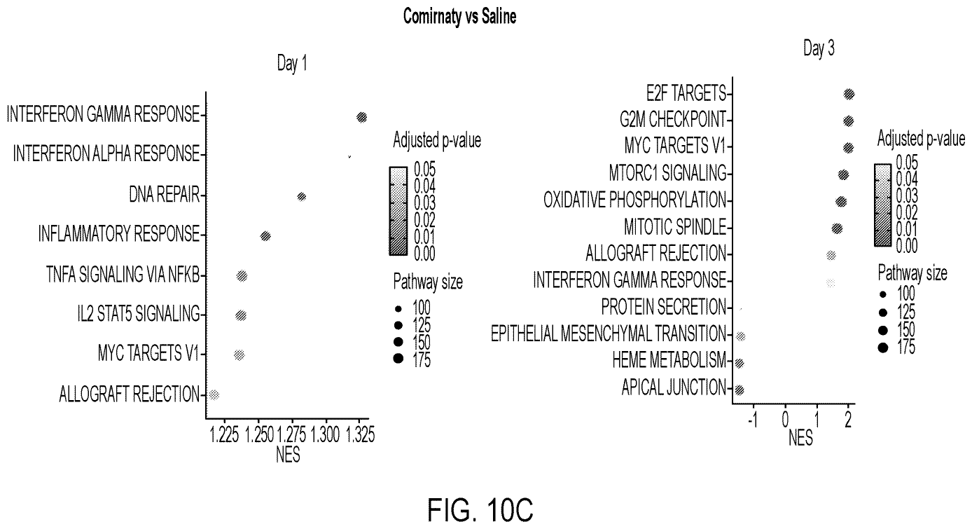

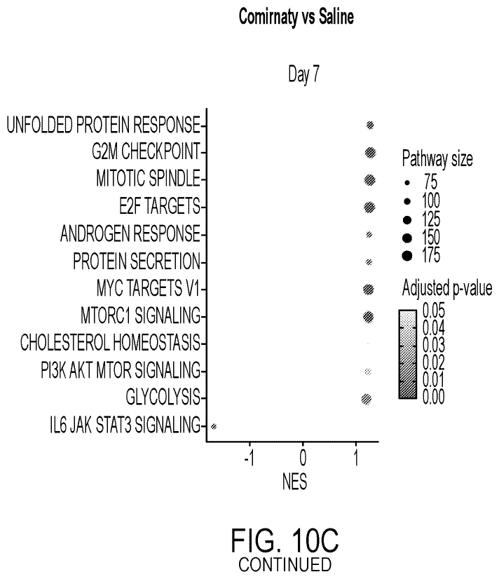

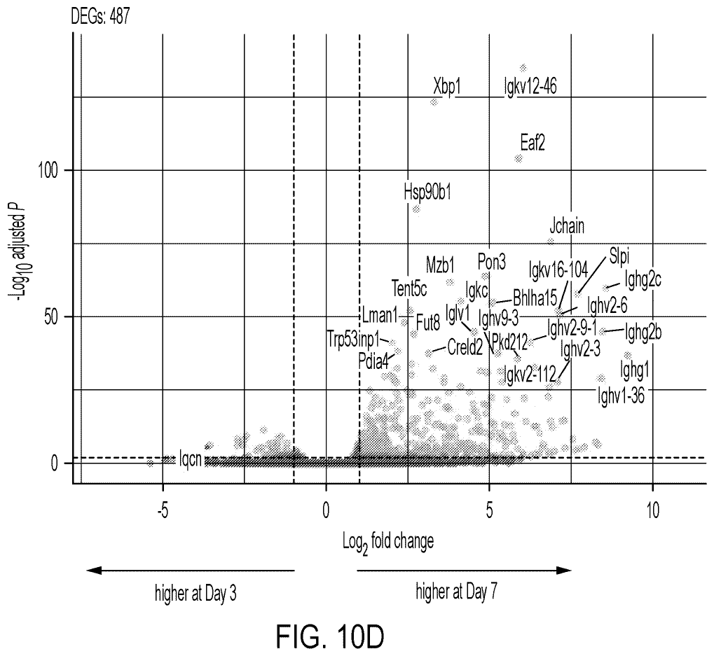

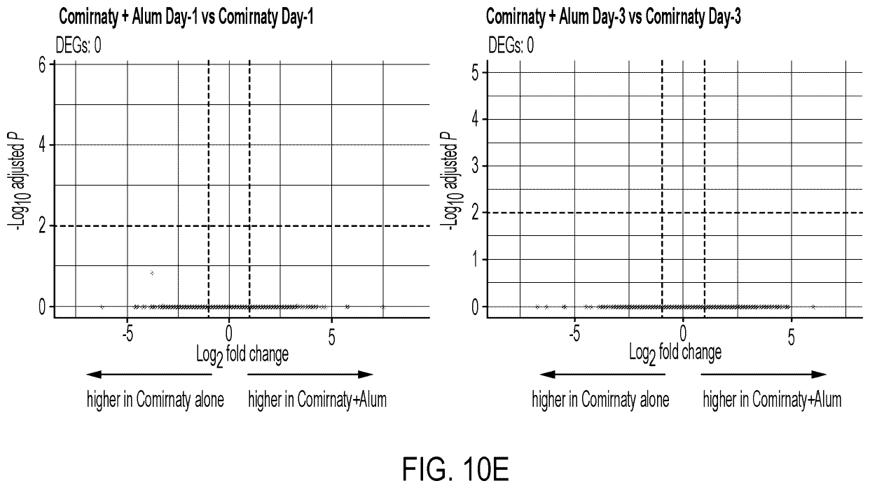

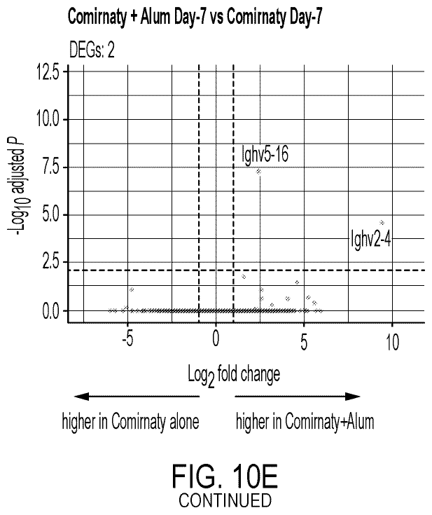

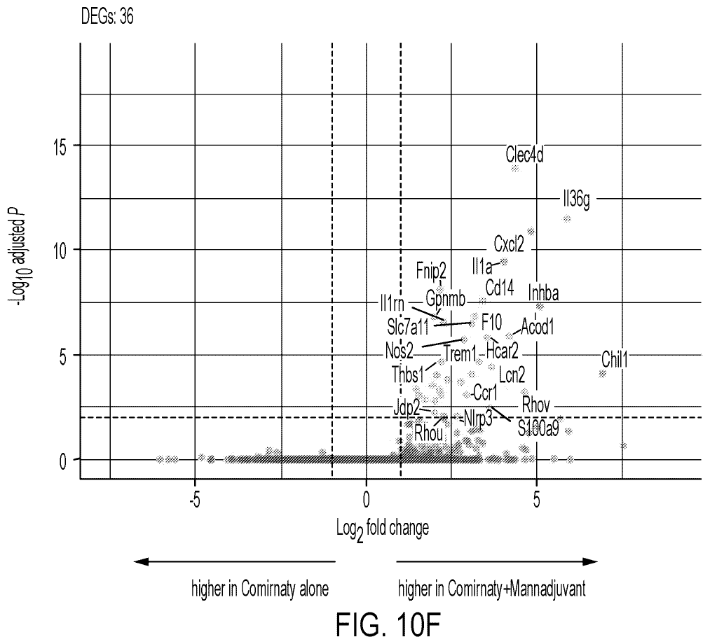

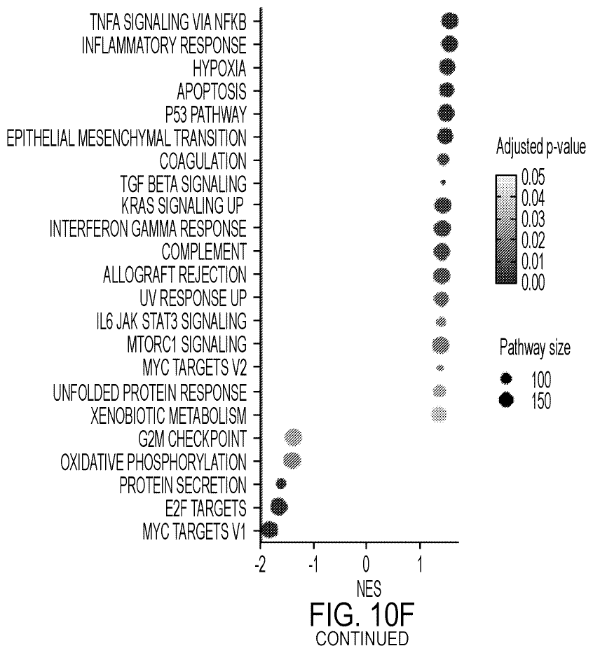

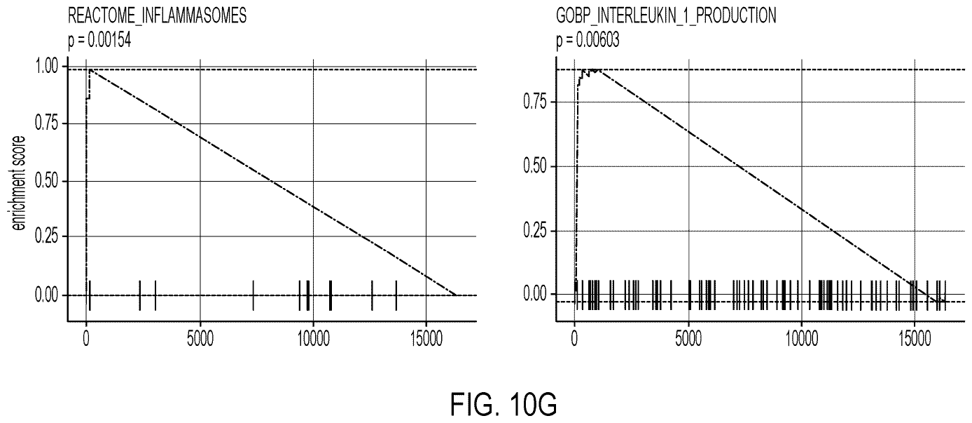

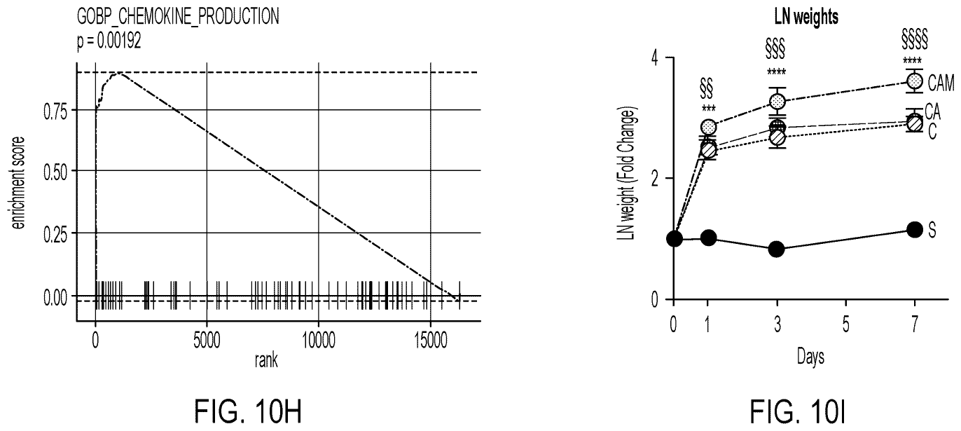

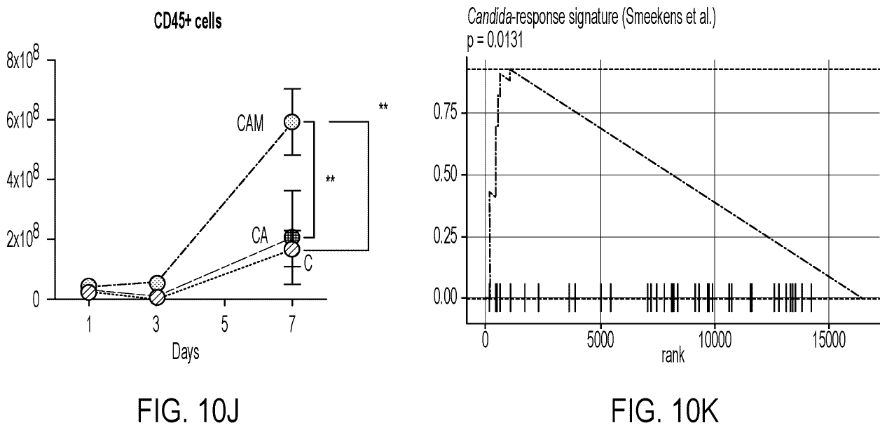

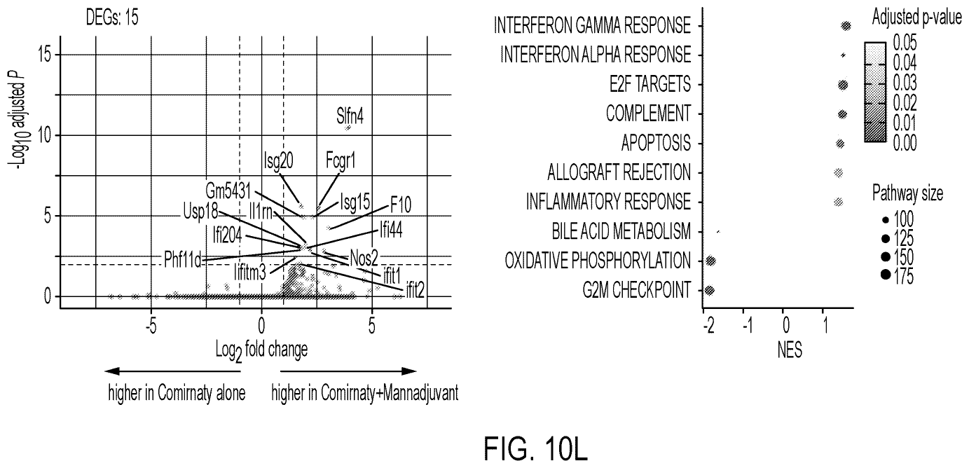

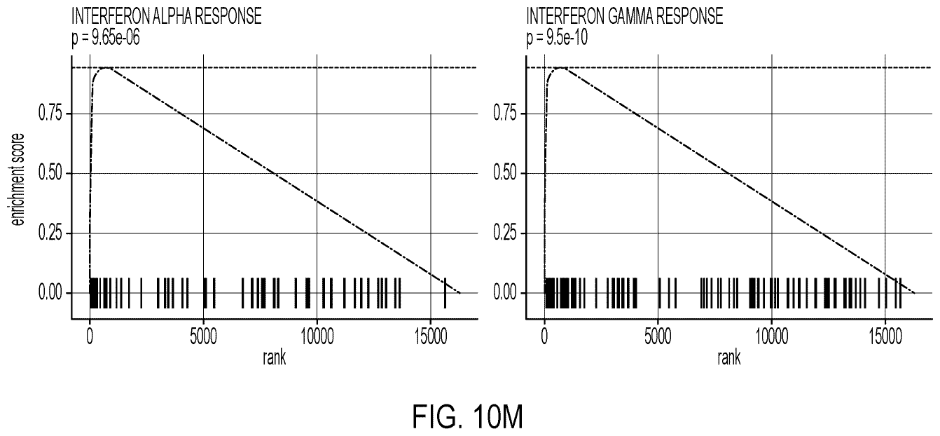

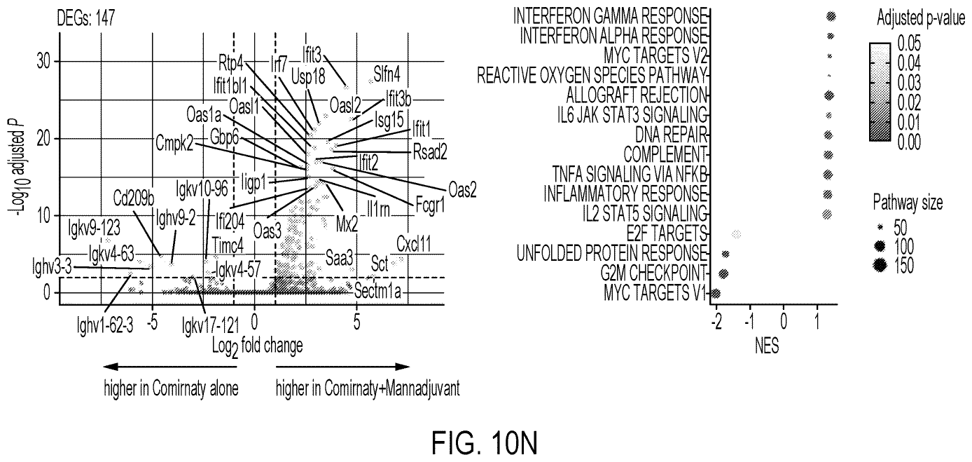

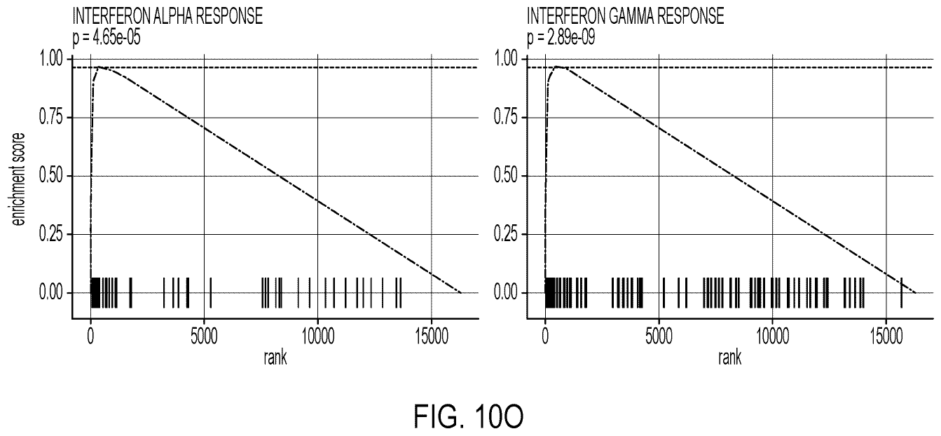

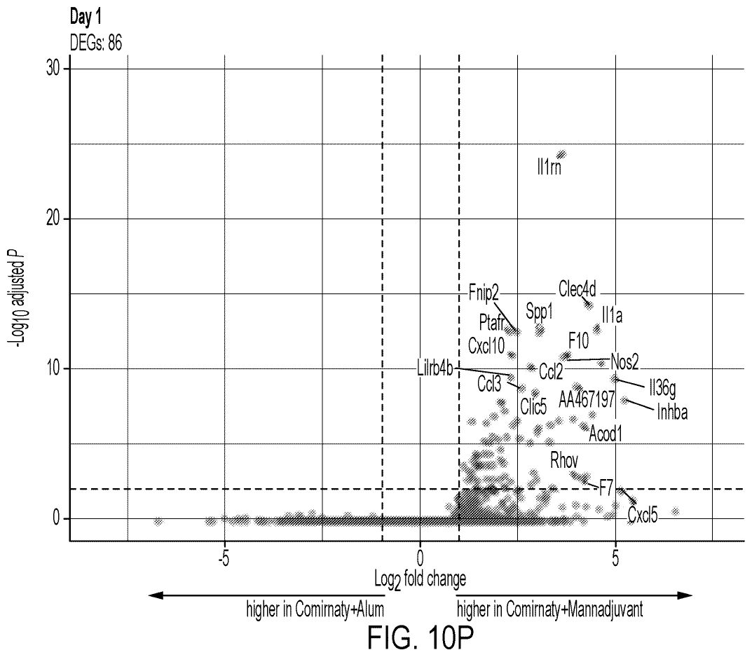

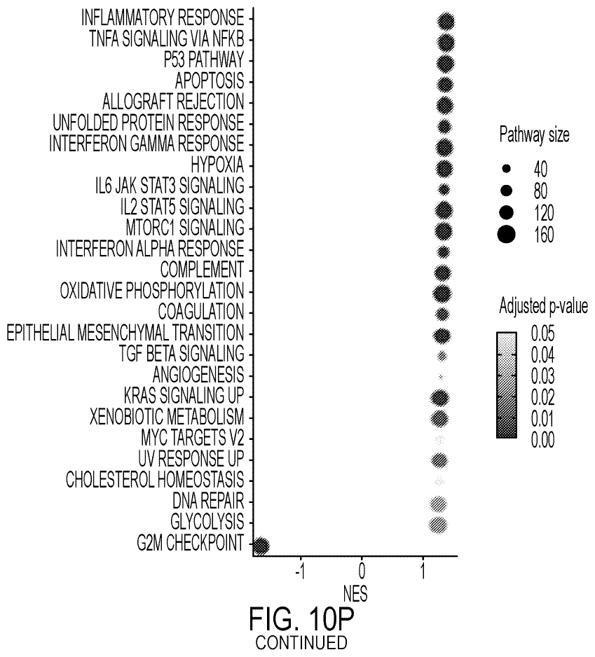

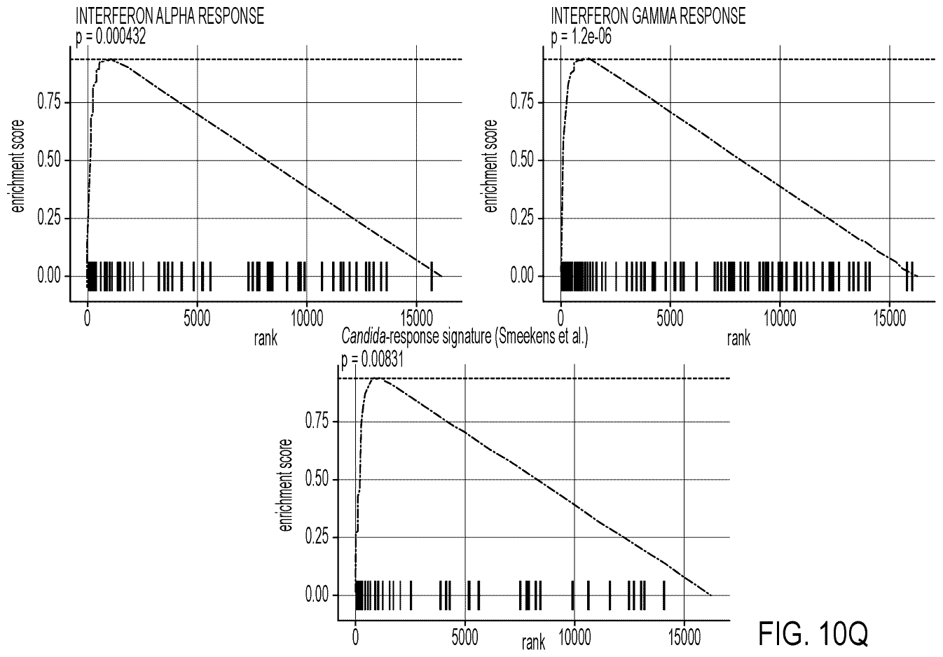

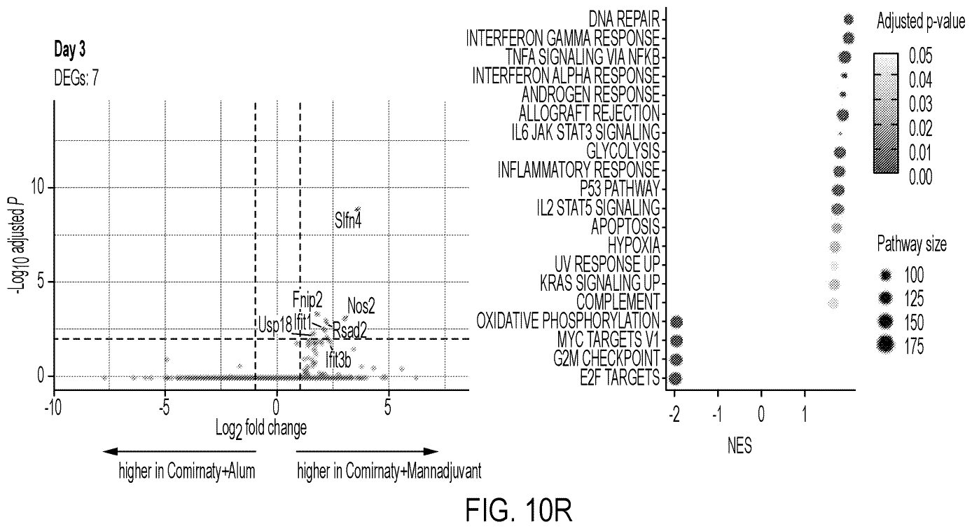

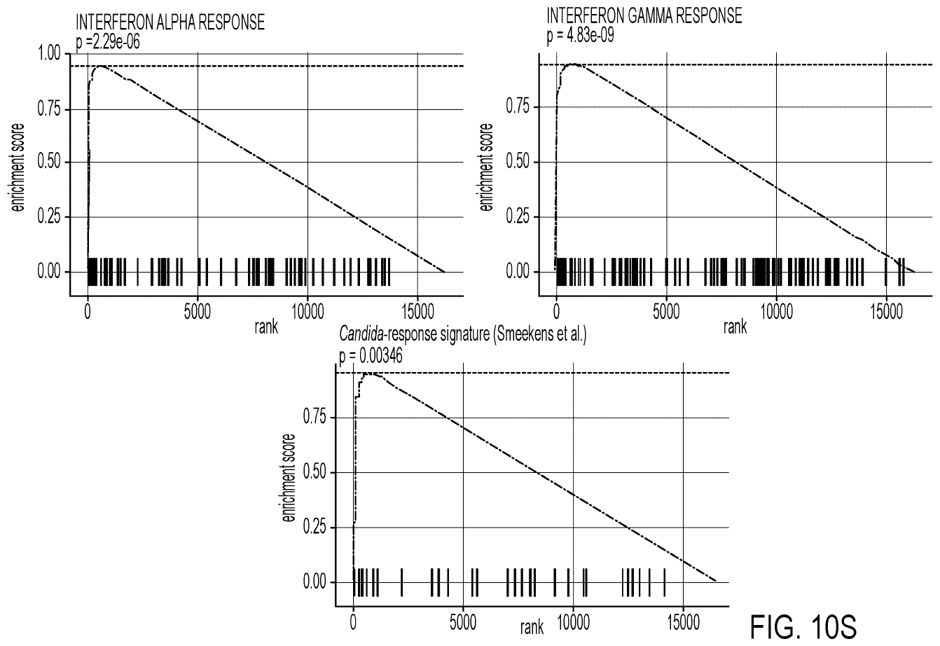

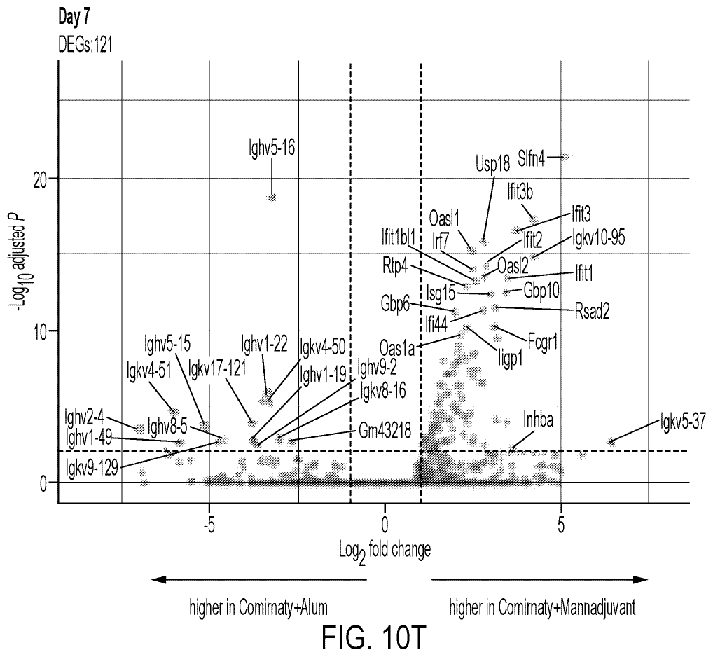

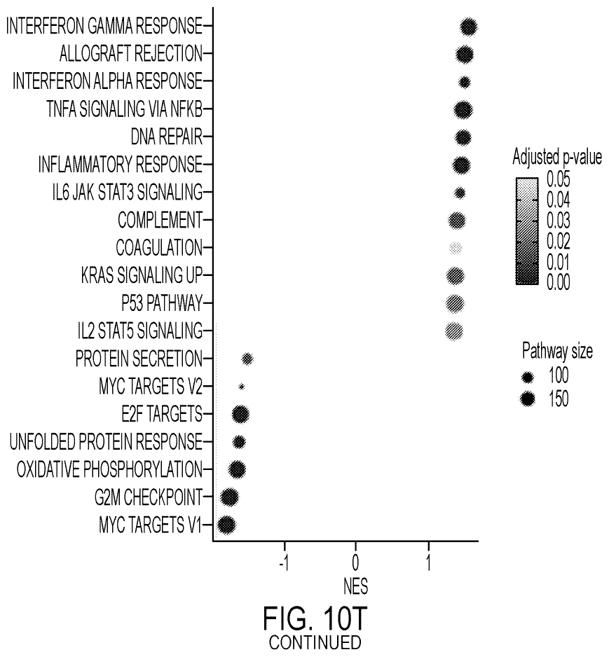

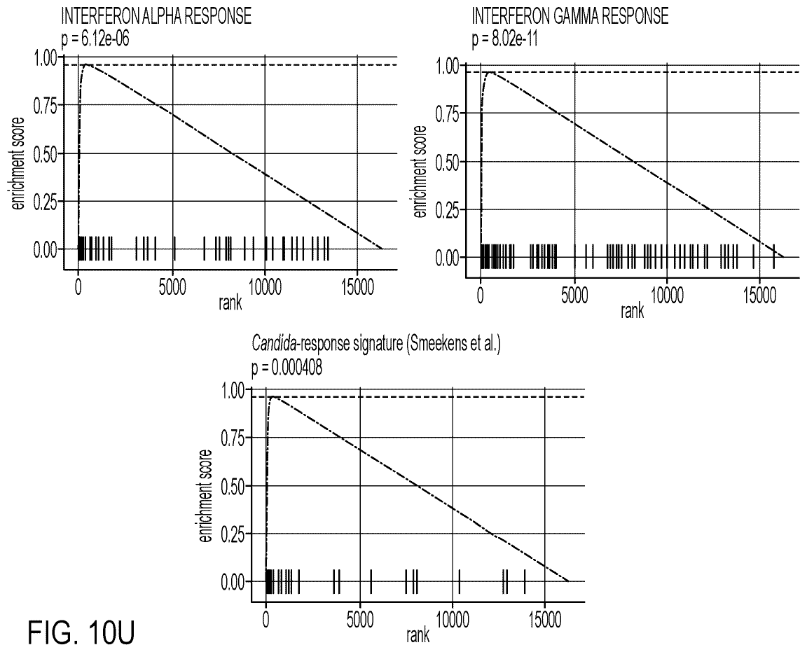

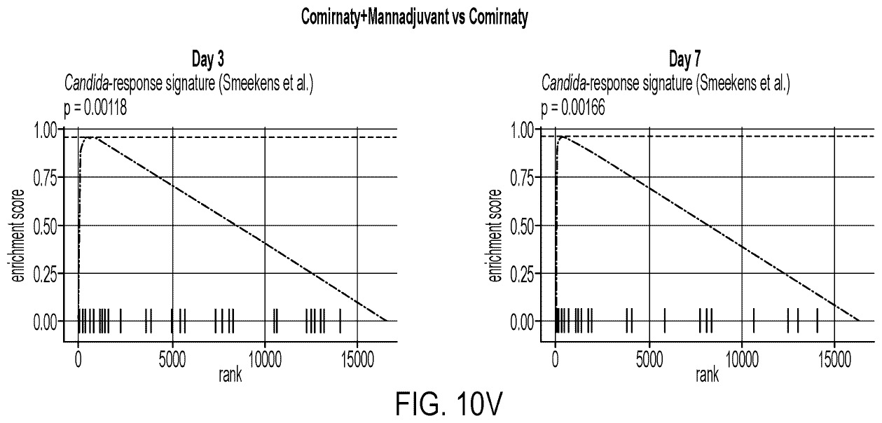

Principal component analysis (PCA) revealed a dynamic and profound impact on gene transcription in immunized mice (FIG. 10A). The transcriptional programs activated in the draining lymph node of mice immunized with Comirnaty alone, or Comirnaty and alum, were overlapping at all time points. In contrast, the PCA showed that mice immunized with Comirnaty and alum-mannan ended in a discrete space compared to the other two treatments. The transcriptional programs activated in the draining lymph node at day 3, compared to da 1, regressed toward the transcriptional landscape of saline-injected control mice. This behavior suggested that a profound transcriptional reprograming associated with the initial inflammatory burst is followed by transcriptional programs that are more similar to homeostasis. Seven days post immunization the PCA showed a profound change compared to day 0, 1, or 3 for all treatments, suggesting that changes associated with the activation of the adaptive immune response profoundly impacted overall gene transcription in the draining lymph node. In keeping with these hypotheses, the PC-driving genes for both components were high at day 1 compared to day 3, while a completely different set of genes was driving the two PCs at day 7 (FIG. 10B). Of note, while genes that characterized day 1 were almost completely switched off at day 3 in mice that received Comirnaty alone or with alum, they were still present in mice that received Comirnaty and alum-mannan both 3- and 7-days post-immunization.

Next, the transcriptional programs and genes regulated over time after immunization with Comirnaty were evaluated. Pro-inflammatory programs were highly upregulated one day after immunization (FIG. 10C). The transcriptional programs of the draining lymph node 3-days post-immunization were characterized by proliferative pathways that may represent the very initial activation of the adaptive arm of the immune system. Pathways suggestive of B cell activation, such as proliferation and protein secretion pathways, were significantly upregulated 7-days post-immunization. Of note, the most upregulated genes 7-days after the initial immunization, compared to day 3, were represented by B cell receptor-associated genes (FIG. 10D). In keeping with the PCA, the volcano plots of differentially expressed genes (DEGs) of the draining lymph nodes from mice immunized with Comirnaty alone or with alum showed only minor differences (FIG. 10E).

Next, the transcriptional programs that were differentially regulated in mice that were immunized with Comirnaty or with Comirnaty and alum-mannan were evaluated. One day after immunization, mice that received Comirnaty with alum-mannan, compared to mice that received Comirnaty alone, showed a significant upregulation of pathways associated with the inflammatory process (FIG. 10F). Genes such as Nlrp3, Il1m, and Il1a were among the most upregulated in mice immunized with Comirnaty and alum-mannan. compared to mice that only received Comirnaty. GSEA confirmed upregulation of gene sets involved in inflammasome activation and IL-1 production (FIG. 10G). Similarly, the GSEA also revealed the enrichment in chemokine production (FIG. 10H). In keeping with this, starting one day after immunization, a significant increase in draining lymph node expansion and immune cell accrual was found in mice injected with Comirnaty and alum-mannan, compared to mice that only received Comirnaty or Comirnaty with alum (FIGS. 10I-10J). Mice that received alum-mannan also showed an increased signature previously identified in humans infected with C. albicans (FIG. 10K).

Three days post-immunization, mice that received Comirnaty and alum-mannan, compared to mice that were immunized with Comirnaty alone, showed increased interferon-driven responses (FIGS. 10L-10M), that were extended up to 7-days post immunization (FIGS. 10N-100). A similar trend was also present when comparing mice that received Comirnaty and alum-mannan, or that were immunized with Comirnaty and alum (FIGS. 10P-10U). Significantly, increased C. albicans signaling was maintained 3- and 7-days post-immunization (FIG. 1V).

Overall, this Example demonstrates that alum-mannan profoundly changes the transcriptional programs initiated by Comirnaty. The activity of the mannan increases the magnitude of inflammasome-mediated responses, and extends type I and type II interferon signaling, suggesting that these pathways underline the changes in the GC response driven by alum-mannan.

### Example 9: Alum-Mannan Controls Inflammatory Programs in Human Phagocytes

The Examples above have illustrated the capacity of glycan adjuvantation to modify the inflammatory programs initiated by Comirnaty in mice, leading to the expansion of GC formation and to a more diverse repertoire of antibodies that can neutralize multiple SARS-CoV-2 variants of concern. The Examples have also demonstrated that these changes are associated with increased inflammasome- and interferon-related transcriptional programs.

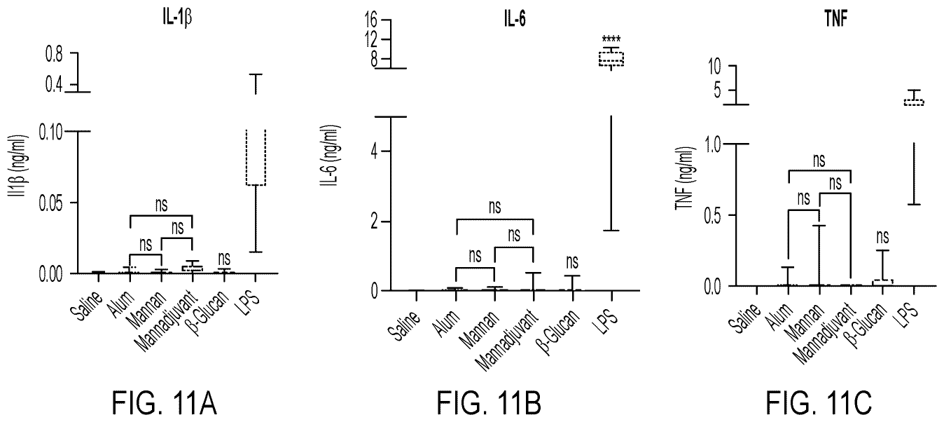

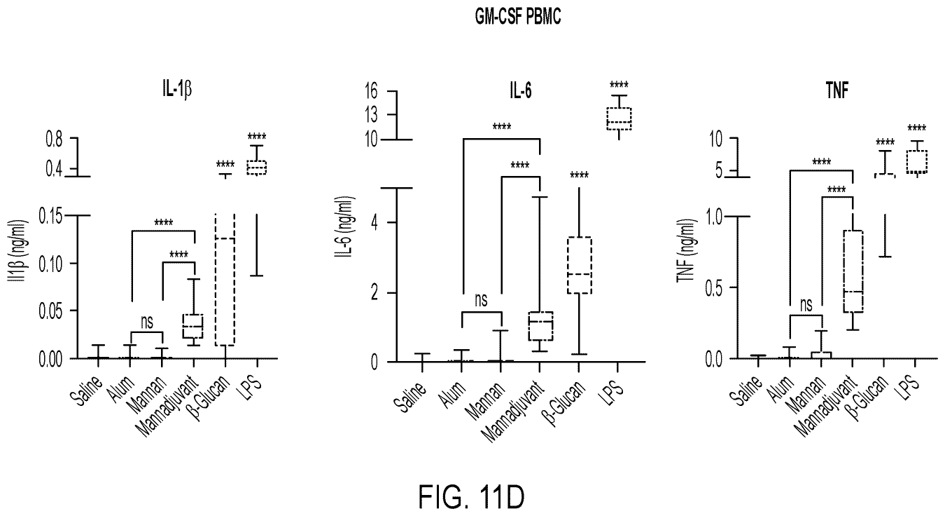

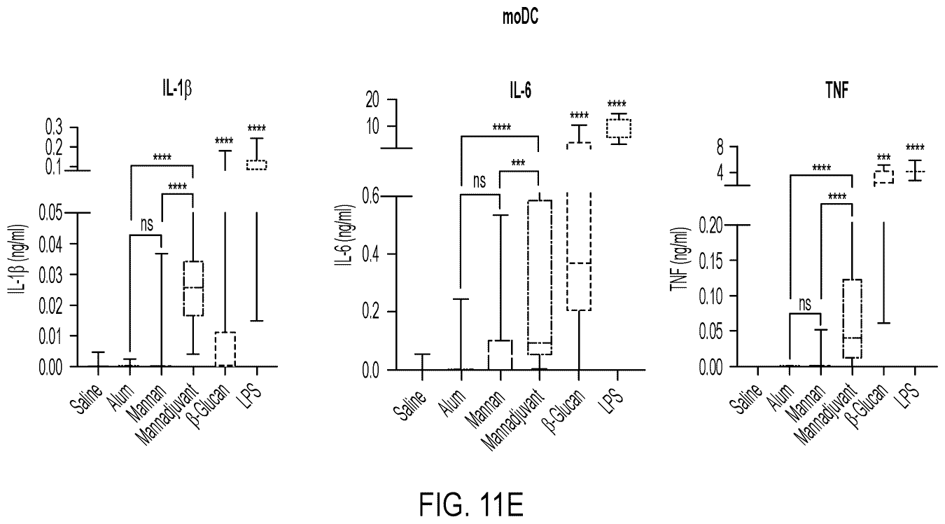

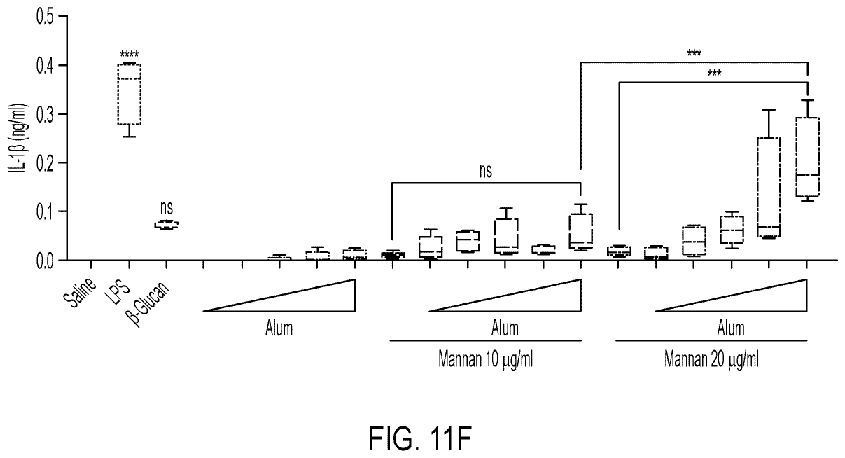

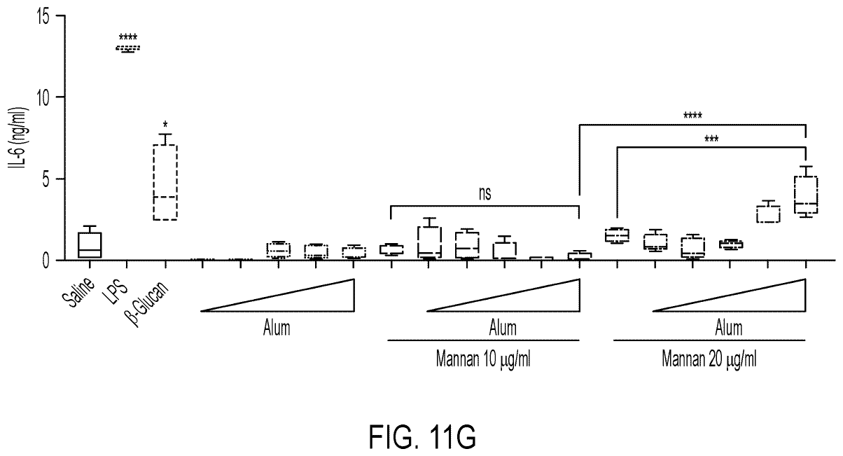

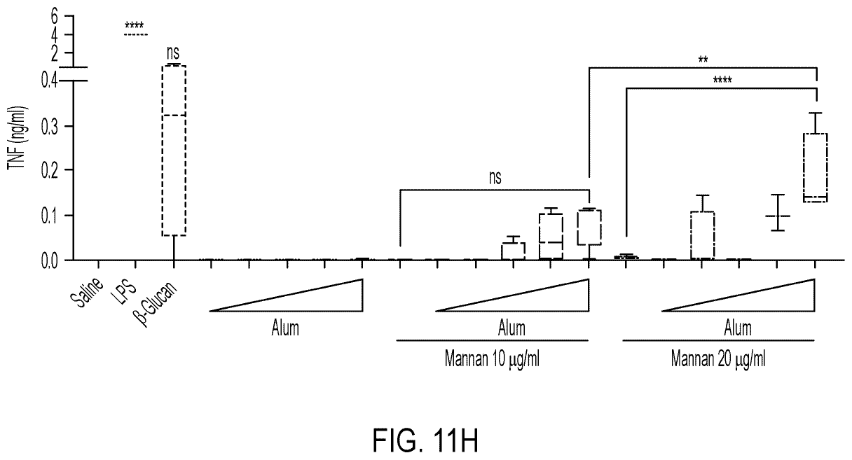

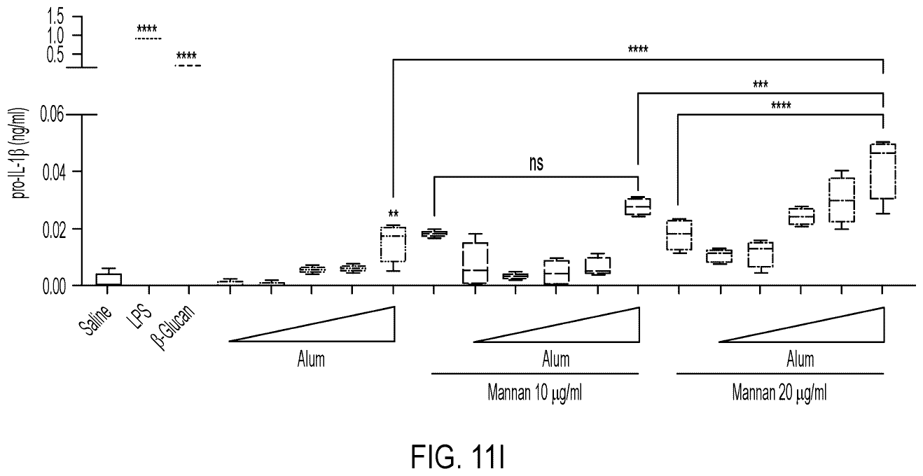

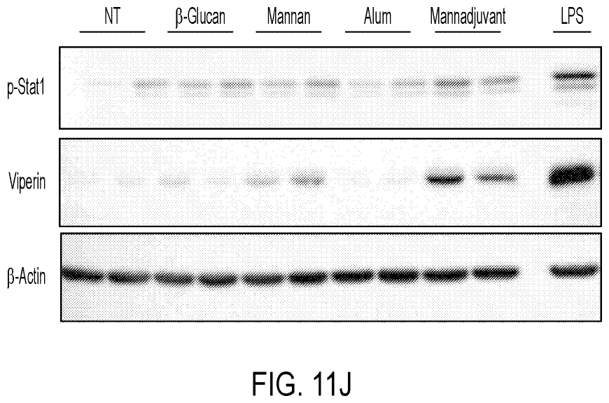

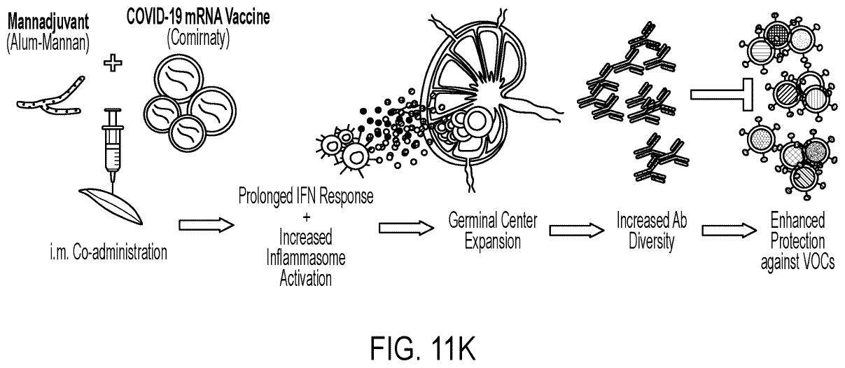

To translate these findings to humans, the capacity of alum-mannan to elicit responses in peripheral blood mononuclear cells (PBMCs) of healthy donors was tested. GM-CSF is fundamental to make immune cells responsive to fungal ligands. It was confirmed that, in the absence of GM-CSF, PBMCs did not respond to fungal PAMPs, while they responded to the bacterial PAMP lipopolysaccharide (FIGS. 11A-11C). In agreement with previous findings that showed only particulate, but not soluble, fungal ligands induce Dectin signaling, PBMCs treated for 16 hours with GM-CSF acquired the capacity to respond to the mannan only when formulated with alum, while they efficiently produced pro-inflammatory cytokines in response to LPS as well as to particulate β-glucans (FIG. 11D). Similar results were obtained when monocyte=−derived human dendritic cells (DCs) were differentiated in the presence of GM-CSF (FIG. 11E). Overall, these data demonstrate that alum-mannan acts on human PBMCs and DCs to drive a potent inflammatory response.

The ratio between mannan and alum determines the amount of mannan bound to alum, and the mannan that remains soluble. The admixture of alum-mannan has been designed to obtain a 1:1 ratio between soluble and particulate mannan. This ratio of soluble and particulate mannan allows the in vivo targeting of both the draining lymph node and the periphery, enhancing the potency of this glycan formulation. To maximize the response of human cells in vitro, a dose response experiment was performed to assess whether different ratios between the alum and mannan can affect the immune response of human cells. The results demonstrated that admixing 20 μg of the mannan and 10 μg of alum significantly increased the release of cytokines by GM-CSF-treated PBMCs, as well as the levels of cell-associated pro-IL-1β (FIGS. 11F-11I).

It was previously shown that moue phagocytes produce pro-inflammatory cytokines only in response to alum-mannan, while type I interferons were also induced by the soluble mannan. In keeping with the mouse data, GM-CSF-treated PBMCs were found to efficiently activate STAT-1 and produce the interferon-stimulated gene RSAD2 (encoding Viperin) when stimulated with the soluble manna. The activation of the interferon axis was further increased with the alum-mannan admixture was utilized (FIG. 11J)

Overall, these data demonstrate that an alum-mannan admixture activates human cells and that, in mice, it can be used as an adjuvant for mRNA-based vaccines to potentiate the protection against SARS-CoV-2 variants of concern. In particular, the data shows that admixing alum, mannan, and Comirnaty expands follicular T cells and GC B cells, as well as B memory cell formation, leading to a more diverse repertoire of antibodies that persist for a longer period of time, protecting against multiple variants of SARS-CoV-2 with high escape capacity (FIG. 11K).

### Example 10: Konjac Glucomannan can be Utilized to Create FDCs that Incorporates Konjac Glucomannan, Alum, and Comirnaty

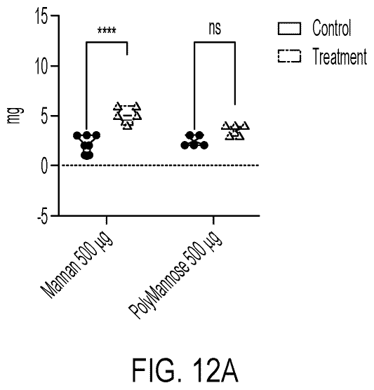

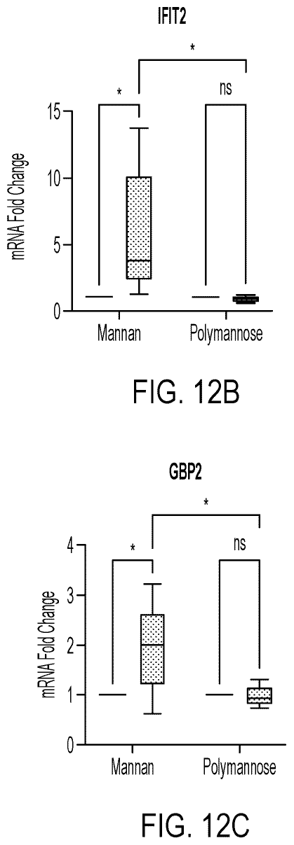

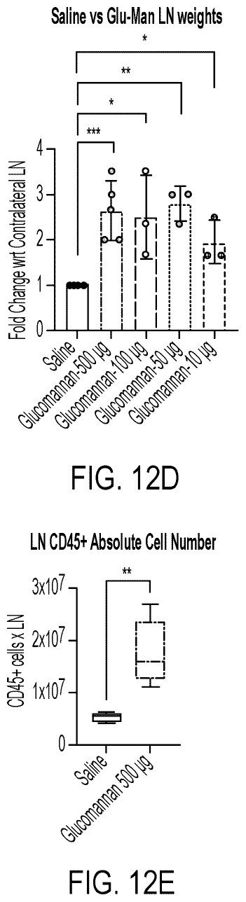

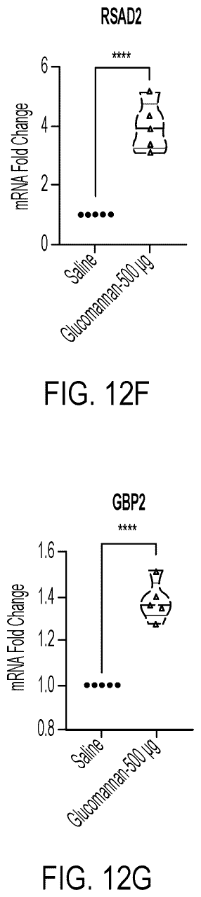

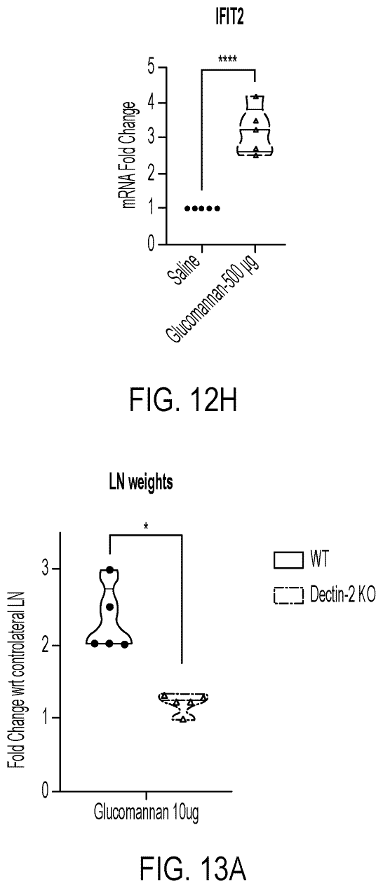

It was previously shown that the immunostimulatory properties of alum-mannan depend on the activation of Dectin-2 and of its downstream interferon signaling capacity. To identify additional Dectin-2 ligands that mirror the immunostimulatory properties of C. albicans-derived mannan, a synthetic polymannose was initially evaluated for its ability to activate Dectin-2. However, compared to C. albicans-derived mannan, the synthetic polymannose did not induce lymph node expansion of interferon signaling (FIGS. 12A-12C). It was reasoned that this could be due to a difference in structure between the synthetic polymannose and C. albicans-derived mannan, which is mostly composed of a backbone of β-1,4-linked to d-mannose residues).

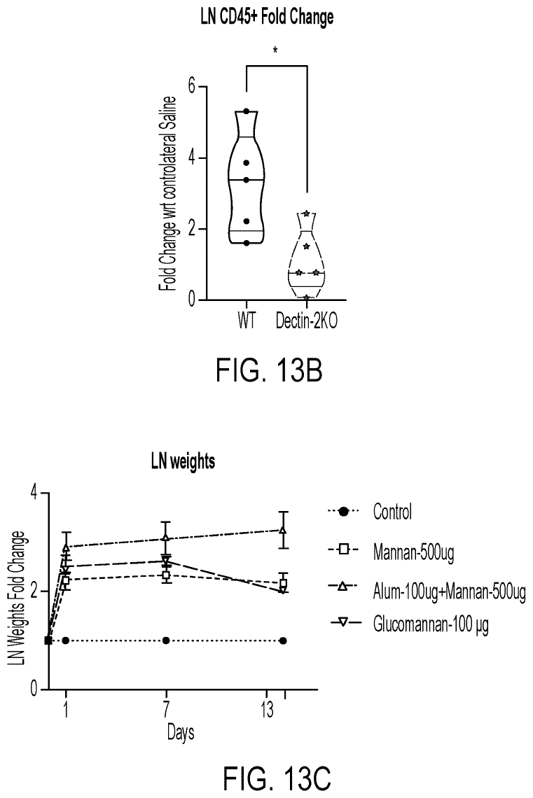

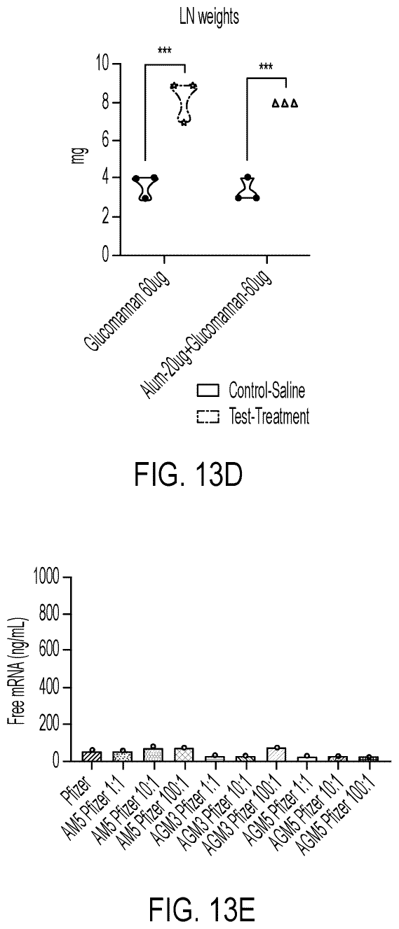

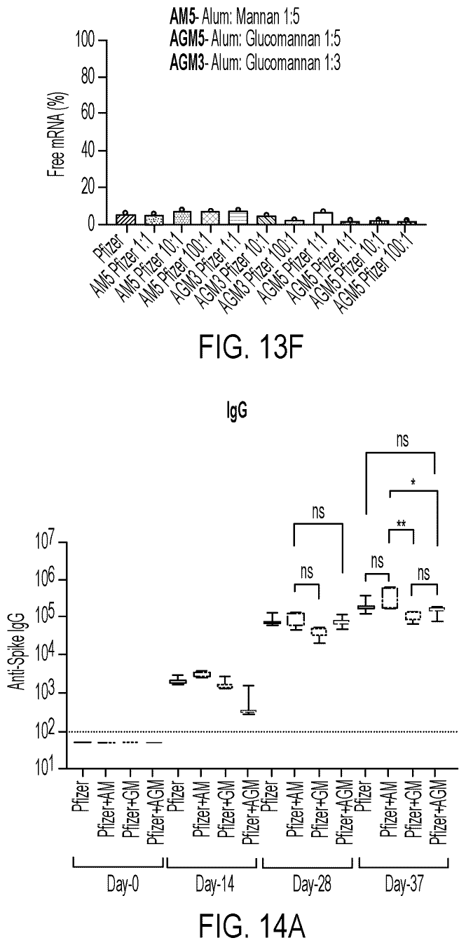

Thus, a Konjac glucomannan having a more similar structure to C. albicans-derived mannan was evaluated for its ability to activate Dectin-2. Mice were injected intradermally with 10 μg, 50 μg, 100 μg, or 500 μg Konjac glucomannan and saline was injected contralaterally. Their draining lymph nodes were collected and weighed 24 hours post-administration. The results show that Konjac glucomannan is able to induce lymph node expansion in a range of doses that varies between 10-500 μg/mouse (FIG. 12D). Konjac glucomannan draining lymph nodes also showed immune cell accrual (FIG. 7E) as well as interferon signaling induction (FIGS. 12F-12H). It was also demonstrated that the activity of Konjac glucomannan was dependent on Dectin-2 signaling, as lymph node expansion 24 hours post-intradermal administration of Konjac glucomannan was only observed in wild-type mice and not Dectin-2-deficient mice (FIGS. 13A-13B) and that lymph node expansion was sustained over time, similar to C. albicans-mannan and alum-mannan (FIG. 13C).

Next, Konjac glucomannan formulated with alum was evaluated for its capacity to initiate lymph node responses. Mice were administered saline, 60 μg Konjac glucomannan, or 60 μg Konjac glucomannan with 20 μg alum and their draining lymph nodes were collected 24 hours post-administration. The results demonstrated the Konjac glucomannan formulated with alum maintains the capacity to initiate lymph node responses, similar to C. albicans-derived mannan (FIG. 13D). Finally, Comirnaty (BNT162b2 mRNA, Pfizer-BioNTech) was admixed with alum-mannan (1:5 weight ratio of alum:mannan) or alum-glucomannan (1:5 weight ratio of alum-glucomannan or 1:3 weight ratio of alum-glucomannan) and incubated for 24 hours at 4° C. (see Table 2). The free mRNA in solution was determined using Quant-it™ RiboGreen RNA Assay it (Thermo Fisher Scientific). Released mRNA was quantified (FIG. 13E) and evaluated as a percentage of total mRNA (FIG. 13F), showing that all FDCs exhibited a minimal degree of mRNA release (<10%) even when the alum-mannan complex or alum-glucomannan complex was in 100-fold excess. Together, these results indicate that Comirnaty is physically and chemically stable in the presence of excess alum-mannan complex or alum-glucomannan complex, both of which can be admixed to Comirnaty without destabilizing the lipid shell of Comirnaty.

TABLE 2
Weight ratio of Comirnaty to various alum-mannan and alum-glucomannan complexes.

| | | | 1:5 weight ratio | 1:5 weight ratio | 1:3 weight ratio |
|---|---|---|---|---|---|
| | Comirnaty | | alum:mannan | alum:glucomannan | alum:glucomannan |
| Description | (Pfizer) | | (AM5) | (AGM5) | (AGM3) |
| Pfizer | 1 | : | 0 | 0 | 0 |
| AM5 Pfizer 1:1 | | | 1 | 0 | 0 |
| AM5 Pfizer 10:1 | | | 10 | 0 | 0 |
| AM5 Pfizer 100:1 | | | 100 | 0 | 0 |
| AGM3 Pfizer 1:1 | | | 0 | 0 | 1 |
| AGM3 Pfizer 10:1 | | | 0 | 0 | 10 |
| AGM3 Pfizer 100:1 | | | 0 | 0 | 100 |
| AGM5 Pfizer 1:1 | | | 0 | 1 | 0 |
| AGM5 Pfizer 10:1 | | | 0 | 10 | 0 |
| AGM5 Pfizer 100:1 | | | 0 | 100 | 0 |

### Example 11: The FDCakgm Vaccine Induces Potent Responses Against SARS-CoV-2 WA1 and B.1.1.529

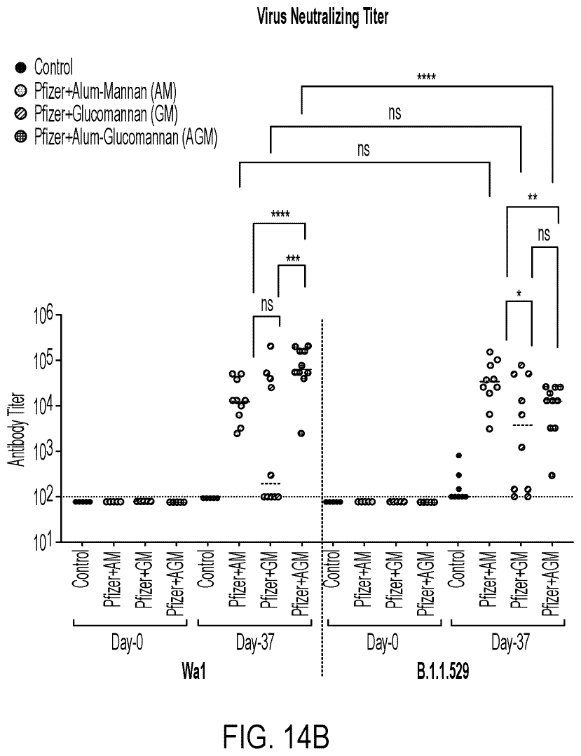

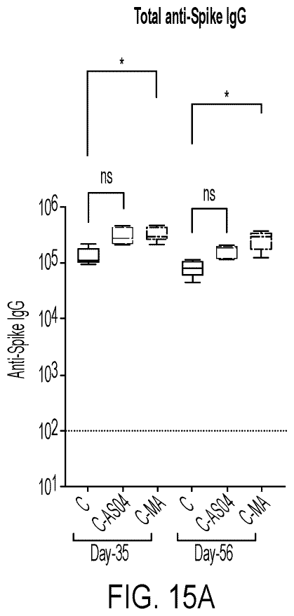

The capacity of Comirnaty (BNT162b2 mRNA, Pfizer-BioNTech) alone or formulated with alum-glucomannan (FDCakgm vaccine) or glucomannan (FDCkgm) to induce anti-Spike antibodies was evaluated. Mice were immunized using a prime-boost schedule and antibody levels were detected in mouse sera before priming (day 0), 14 days post-priming (day 14), 14 days post-boosting (day 28), or 23 days post-boosting (day 37). All formulations were found to efficiently induce anti-Spike IgG by day 28, an effect which persisted to day 37 (FIG. 14A). Next, the capacity of mouse sera derived from mice immunized with Comirnaty alone or with glucomannan or alum-glucomannan to neutralize SARS-CoV-2 WA1 or SARS-CoV-2 B.1.1.529. The data shows that admixtures of Comirnaty with alum-mannan (FDC) or alum-glucomannan (FDCakgm) had a superior capacity to neutralize either SARS-CoV-2 WA1 and SARS-CoV-2 B.1.1.529 (FIG. 14B). Overall, these data support the use of FDC or FDCakgm as a means to increase the protection induced by mRNA vaccines against ancestral SARS-CoV-2 and other variants of concern.

### Example 12: Comparison of Comirnaty Efficacy with Different Adjuvants

This Example compares the efficacy of the alum-mannan adjuvantation system with AS04. AS04 is a toll-like receptor-based adjuvant, consisting of aluminum hydroxide and monophosphoryl lipid A. Mice were immunized intramuscularly with Comirnaty (BNT162b2 mRNA; Pfizer-BioNTech) alone, or with AS04 or alum-mannan on day 0 and day 14. Mouse sera was collected on day 35 and day 56 and evaluated for anti-Spike IgG levels and neutralizing titers, and draining lymph nodes were collected on day 56.

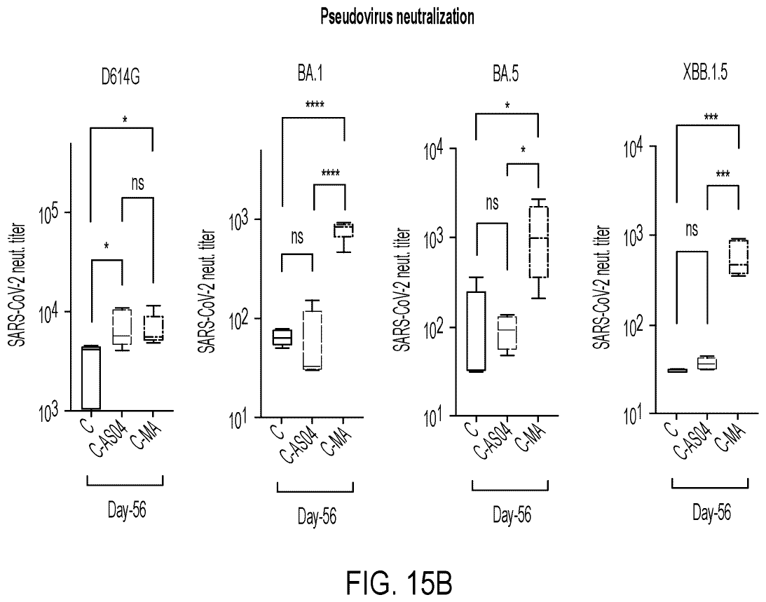

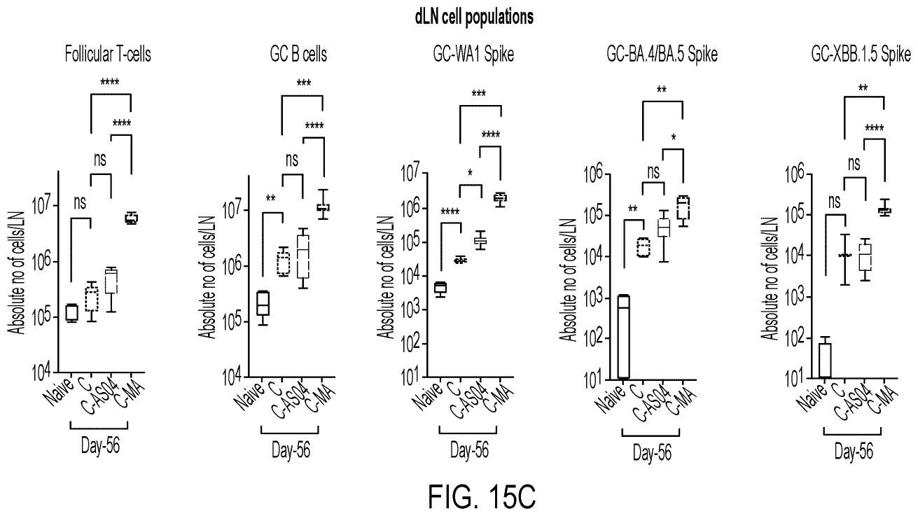

Quantification of anti-Spike IgG levels in mice on days 35 and 56 post-immunization reveled that the alum-mannan adjuvantation system significantly increased total anti-Spike IgG levels (FIG. 15A), significantly boosted the neutralization of the BA.5 and XBB.1.5 variants of concern (FIG. 15B), and also significantly boosted the expansion of follicular T cells germinal center B cells, and germinal center B cells that express a B cell receptor capable of recognizing different variants of concern (FIG. 15C), as compared to immunization with Comirnaty alone or with AS04.

These data demonstrate the unique features of enhancing the activity of Comirnaty with alum-mannan.

### Example 13: Adjuvantation with Alum-Mannan Expands and Prolongs the Presence of Antibodies in Mouse Sera

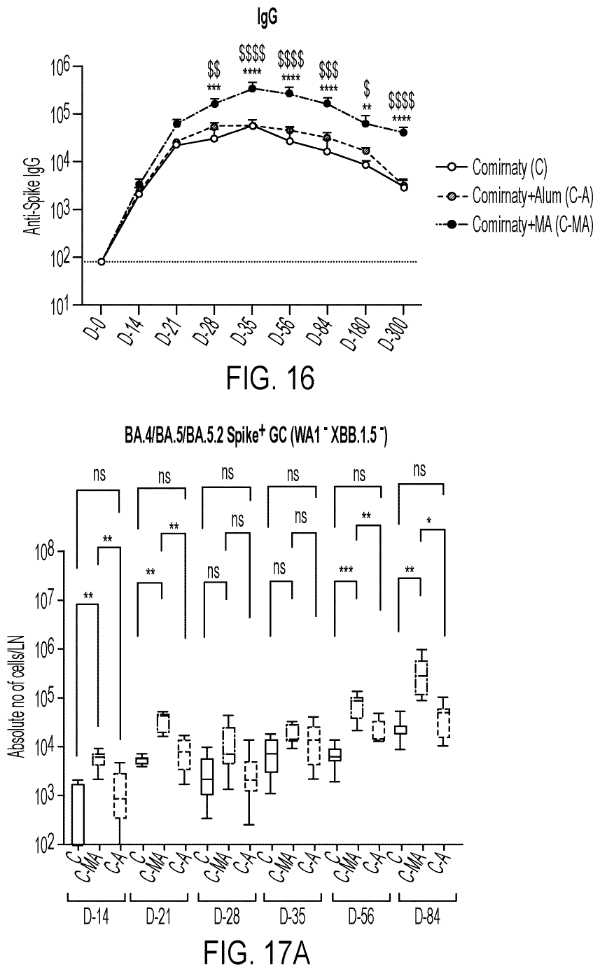

Mice were immunized intramuscularly on day 0 and day 14 with Comirnaty (BNT162b2 mRNA, Pfizer-BioNTech) alone or with alum or alum-mannan. Mouse sera was collected on pre-immunization on day 0 and on days 14, 21, 28, 35, 56, 84, 180 and 300 post-immunization and evaluated for anti-Spike IgG levels. The levels of anti-Spike antibodies in the serum of mice that received Comirnaty with alum-mannan after 300 days were found to be as high as the peak antibody levels induced in mice immunized with Comirnaty after 5 weeks (FIG. 16). This observation demonstrates the capacity of alum-mannan to expand and prolong the presence of antibodies in the serum of mice immunized with Comirnaty.

### Example 14: Adjuvantation with Alum-Mannan “Shifts” Antigenic Sin

Beyond a short duration of the antibody levels and the protection elicited by mRNA-based vaccine platforms, another major limitation of current mRNA-based vaccines is the induction of immune imprinting for the original antigen. This strongly diminishes the possibility to induce protection against new variants of concern. In this Example, the capacity of alum-mannan to “shift” the antigenic sin was tested.

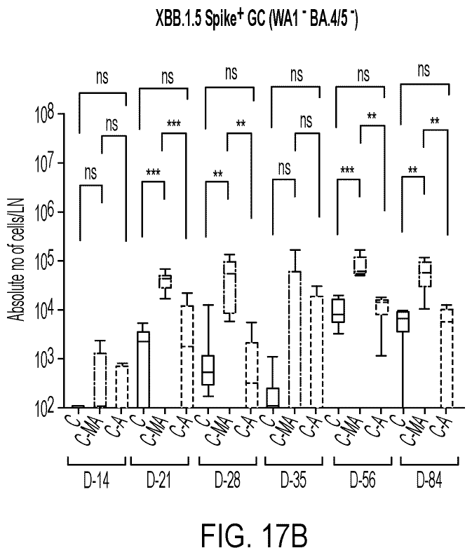

Mice were immunized on day 0 and day 14 with Comirnaty (BNT162b2 mRNA, Pfizer-BioNTech) alone or with alum or alum-mannan. Mouse sera was collected on days 14, 21, 28, 35, 56, and 84 post-initial immunization. Mice immunized with Comirnaty and alum-mannan showed a significant expansion of germinal center B cells that express a B cell receptor that uniquely recognizes the Spike protein of SARS-CoV-2 BA.4/BA.5 (FIG. 17A) and SARS-CoV-2 XBB.1.5 (FIG. 17B). These data suggest that alum-mannan adjuvantation allows the formation of more diverse B cell clones that can react with new epitopes contained in the Spike proteins of new variants of interest, and thus has the potential to overcome antigenic sin.

### Example 15: Methods

Cell culture. VeroE6 cells (ATCC, CRL-1586) and 293 T cells were cultured in Dulbecco's modified Eagle Medium (DMEM; Thermo Fisher Scientific, Waltham, MA, USA) supplemented with 10% Fetal Bovine Serum (FBS; Thermo Fisher Scientific) and 1% penicillin/streptomycin (Lonza, Basel, Switzerland). 293T cells stably overexpressing ACE2 and TMSPPR2 were maintained in DMEM supplemented with 10% FBS, 1% penicillin/streptomycin and 1% sodium pyruvate (Thermo Fisher Scientific).

Viruses. Samples of SARS-CoV-2 (WA1) were obtained from the Centers for Disease Control and Prevention (CDC) following isolation from a patient in Washington State (Biodefense and Emerging Infections Research Resources Repository (BEI Resources), Isolate USA-WA1/2020, NR-52281). SARS-CoV-2 variants are B.1.1.529/BA.1, BA.4 (BEI NR-56806), BA.5 (BEI NR-58620), and XBB.1.5 (BEI NR-59104). All SARS-COV-2 stocks were amplified in Vero/TMPRSS2 cells and sequence-verified before use.

Preparation of alum-mannan complex. Mannan was isolated from Candida albicans SC5314 as previously described (Kruppa et al., 2011; Lowman et al., 2011) and quality controlled by nuclear magnetic resonance spectroscopy (NMR). Alum-mannan complex was prepared by incubating alum (100 μg in 10 μl; Alhydrogel® adjuvant 2%; Invivogen, San Diego, CA, USA) with mannan (500 μg in 25 μl) in 0.9% saline (15 μl) for 30 minutes at room temperature. Unless another weight ratio is specified, mice were injected with a formulation containing 100 μg/ml alum and 500 μg/ml mannan (1:5 alum:mannan weight ratio).

Vaccine formulation. To formulate the alum-mannan complex with SARS-CoV-2 Spike protein trimer or Comirnaty (BNT162b2 mRNA, Pfizer-BioNTech), the volume of saline was reduced to account for the additional volume. Comirnaty and SARS-CoV-2 Spike protein trimer were used at 1 μg per injection.

Immunization and antibody quantification (Examples 1-5). C57BL/6 (wild-type, WT) mice were immunized on day 0 and day 14 (and additionally day 56, in Example 4) by: i) intradermal injection of a pre-fusion stabilized SARS-CoV-2 WA1 Spike trimer (Spike; 1 g/mouse) alone or formulated with alum-mannan; ii) intramuscular injection of Comirnaty (BNT162b2 mRNA, Pfizer-BioNTech; 1 μg/mouse) and intradermal injection of alum-mannan; iii) intramuscular injection of Spike formulated with alum-mannan; or iv) intramuscular injection of Comirnaty and alum-mannan. Blood samples were collected by retroorbital bleeding on day 14 (pre-boost) and day 28, and additionally on days 35, 37, 56, 63, 73, and/or 84 where specified. Serum samples were isolated after centrifugation of blood samples twice at 1500×g for 10 minutes. IgG, IgG1, and IgG2c antibody levels were quantified in serum samples by ELISA using a modified previously described protocol (Borriello et al., 2017). Briefly, high binding flat bottom 96-well plates were coated with 0.5 μg/ml SARS-CoV-2 Spike trimer in PBS, incubated overnight at 4° C., washed once with 0.05% Tween-20 in PBS and blocked with 1% BSA in PBS for 1 hour at room temperature Serum samples were added at an initial dilution of 1:100 in 1% BSA in PBS. To generate 11-point curves 1:4 serial dilutions were performed. Samples were incubated for 2 hours at room temperature. Plates were washed three times with 0.05% Tween-20 in PBS and incubated for 1 hour at room temperature with HRP-conjugated anti-mouse IgG (Southern Biotech, cat #1036-05), IgG1 (Southern Biotech, cat #1071-05), or IgG2c (Southern Biotech, cat #1078-05) antibodies. Finally, plates were washed five times with 0.05% Tween-20 in PBS and developed with tetramethylbenzidine (BD OptEIA Substrate Solution for Spike, BD Biosciences) for 5 minutes, then stopped with 2NH₂SO₄. Optical densities (ODs) were read at 450 nm with SpectraMax iD3x microplate reader (Molecular Devices) and endpoint titers were calculated using as cutoff three times the optical density of the background. Values less than 100 were reported as 80.

SARS-CoV-2 neutralization titer determination. All serum samples were heat-inactivated at 50° C. for 30 minutes to deactivate complement and allowed to equilibrate to room temperature prior to processing for neutralization titer determination. Samples were diluted in duplicate to an initial dilution of 1:5 or 1:10, followed by 1:2 serial dilutions, resulting in a 12-dilution series with each well containing 100 μl. All dilutions were performed in DMEM (quality Biological), supplemented with 10% (v/v) fetal bovine serum (heat-inactivated, MilliporeSigma), 1% (v/v) penicillin/streptomycin (Gemini Bio-products), and 1% (v/v) L-glutamine (2 mM final concentration, Thermo Fisher Scientific). Dilution plates were then transported into a BSL-3 laboratory and 100 μl of diluted SARS-CoV-2 (WA-1, B.1.1.529, BA.4/BA.5, or XBB.1.5) inoculum was added to each well to result in a multiplicity of infection (MOI) of 0.01 upon transfer to tittering plates. A non-treated virus-only control and a mock infection control were included on every plate. The sample-virus mixture was then incubated at 37° C. (5.0% CO₂) for 1 hour before transferring to 96-well titer plates with confluent VeroE6 cells.

24 hours (48 hours for B.1.1.529) after infection cells were fixed with 10% neutral buffered formalin (Sigma) for at least 1 hour (h) at 4° C. as per BSL3 SOP and immunofluorescence stained for the SARS-CoV-2 N protein. Formaldehyde was quenched by incubating cells with 50 mM ammonium chloride in PBS for 15 minutes, prior to permeabilizing the cells with 0.1% Triton-X100 (Sigma) in 0.2% (w/v) bovine serum album (BSA) in PBS (blocking buffer) for 10 minutes. Cells were blocked for 10 minutes with blocking buffer and then incubated with anti-N antibody (Sino Biologicals 40143-R004) for 1 hour (diluted 1:10,000 in blocking buffer). Primary antibody was removed by washing 3 times for 5 minutes each with blocking buffer and cells were then incubated with goat anti-rabbit Alexa Fluor488 (Thermo Fisher Scientific, A11008), diluted 1:2,000 in blocking buffer for 45 minutes. Cells were washed a further 3 times for 5 minutes each with PBS and incubated with Hoeschst 33342 (Thermo Fisher Scientific) diluted 1:2,000 in PBS for 10 minutes. Cells were washed a final 2 times for 5 minutes each with PBS and then imaged with a Celigo high content imager (Nexcelom). Inhibition data was normalized according to the following formula based on cell-only and virus-only controls: % Neutralization={1−((treatment)−(cell only))/((virus-only)−(cell-only)))}×100%. Nonlinear regression analysis was performed on the normalized inhibition data and EC50s were calculated from fitted curves (log [agonist] versus response−variable slope [four parameters]) using GraphPad Prism (GraphPad Software). EC50 value extrapolated outside the dilution range tested were reported as greater than the highest concentration tested or less than the lowest concentration tested.

SARS-CoV-2 Pseudovirus Neutralization Assay. SARS-Cov2 pseudovirus neutralization assay was done as described previously. Briefly, 293T cells were co-transfected with a pHDM vector expressing SARS-CoV-2 spike protein variants, pLenti CMV Puro LUC (w168-1) (Addgene plasmid #17477), and psPAX2 (Addgene, plasmid #12260) with Lipofectamine 3000 (Thermo Fisher Scientific). After 48 hours, supernatant containing pseudoviruses was collected. 293FT cells stably expressing human ACE2 and TMRPSS2 were infected with pseudoviruses diluted in DMEM containing 10% FBS to test titers. For neutralization assays, 2×10⁴ 293FT cells expressing human ACE2 and TMRPSS2 were plated per well of a 96-well flat bottom plate and incubated overnight at 37° C. Subsequently, serum samples collected from immunized mice were serially diluted and incubated with pseudovirus at 37° C. for 1 hour and the mixture was added in duplicates to the cells. After 48 hours, cells were lysed using Promega ONE-Glo luciferase reagent (Promega, Madison, WI, USA), and the luminescence signal was recorded on a Biotek Synergy H1 instrument (Agilent Technologies). Serum titers were determined by nonlinear regression using Prism 10 (GraphPad, La Jolla, CA, USA) of log-transformed luciferase signal and represented as Neutralizing Titer 50 (NT50) (the titer to cause 50% inhibition of infection). The NT50 was set to the limit of detection (LOD) if the initial dilution of serum (1:30) did not reach a neutralization of 50% or if the regression fit was poor (<0.7).

Enzyme-linked immunosorbent spot (ELISPOT) assay. 96 well Multi-screen filter plates (MilliporeSigma, Cat #MSIPS4510) were pre-wetted with 20 μl of 35% ethanol in H2O (v/v) for 1 minute and washed 5 times with PBS followed by coating with 100 μl of anti-mouse IgG (15 μg/ml; SouthernBiotech, Cat #1030-01) overnight at 4° C. On the next day, plates were washed 5 times with PBS and blocked in 200 μL Roswell Park Memorial Institute (RPMI) 1640 medium (Thermo Fisher Scientific, Cat #61870127) supplemented with 10% FBS, 1% penicillin/streptomycin, 1% L-glutamine for 2 hours at 37° C. Freshly isolated bone marrow cells were seeded at 2×10⁵ cells/well after RBC lysis and incubated for 48 hours at 37° C. Medium-containing wells without cells were used as blank. After incubation, medium was decanted, and plates were washed 5 times with PBS followed by addition of 100 μl biotinylated anti-mouse total IgG (Mabtech, Cat #3825-6) or biotinylated Spike protein (Sino Biological, Cat #40589-V27B-B) in 0.5% BSA in PBS for 2 hours at room temperature. Plates were washed 6 times with PBS and subsequently incubated for 1 hour at room temperature with 100 μl of a 1:1000 dilution of Streptavidin-ALP enzyme conjugate (Mabtech, Cat #3310-10) in 0.5% BSA in PBS. After washing with PBS for 5 times, 100 μl of filtered SIGMAFAST™ BCIP®/NBT (Sigma-Aldrich, Cat #B5655) solution was added and plates were incubated at room temperature until distinct spots became visible. Reaction was stopped by washing the plate with dH₂O and allowed to dry completely. Finally, the plate was read on a CTL Immunospot Microanalyzer plate reader (Cellular Technology Limited, Cleveland, OH, USA). No background subtraction was carried out and Spike+ spot numbers were normalized to numbers of total IgG spot numbers.

Preparation of fixed-dose combination (FDC) vaccines composed of Comirnaty and alum-mannan complex. Alum-mannan complex was prepared as described above. The concentration of alum was varied from 1 μg/ml to 100 μg/ml, while keeping the weight ratio constant. Subsequently, Comirnaty (BNT162b2 mRNA, Pfizer-BioNTech; 1 μg/ml mRNA) was added to the alum-mannan complex and incubated at 4° C. for 24 hours. The ratio between Comirnaty and alum-mannan in the FDC vaccines ranged from 1:1 to 1:100. Dynamic light scattering (ZetaPlus Zeta Potential Analyzer, Brookhaven Instruments, Holtsville, NY) was performed to determine the stability of each active pharmaceutical ingredient in the FDC solution.

Determination of non-specific mRNA release from Comirnaty in the FDCs. FDC vaccines were prepared as described above. After 24 hours of incubation, the FDC vaccines were briefly centrifuged using a benchtop centrifuge to remove the alum-mannan complex from the solutions. The resulting solutions were then transferred to a Nunc MicroWell 96-well optical bottom plate and total “free” mRNA concentration in each FDC was quantified by the Quant-it™ RiboGreen RNA Assay Kit (Thermo Fisher Scientific) according to the manufacturer's protocol. Fluorescence measurement was performed using a Synergy HT Multi-Mode Microplate Reader (BioTek Instruments, Winooski, VT).

Mice. 6-8 weeks old female C57BL/6J mice (Jax 00664) were used for experiments and were purchased from The Jackson Laboratory. Mice were housed under specific pathogen-free conditions at Boston Children's Hospital, and all the procedures were approved under the Institutional Animal Care and Use Committee (IACUC) and operated under the supervision of the department of Animal Resources at Children's Hospital (ARCH).

Lymph node dissociation. Draining lymph nodes (brachial or inguinal) were collected, passed through 70 μm filters and resuspended in PBS supplemented with 1% FBS and 1 mM EDTA. Lymph node weight was assessed on an analytical scale prior to further processing. To generate a single cell suspension, lymph nodes were subjected to a total of 3 cycles of incubation at 37° C. for 20-30 (first cycle) or 10 (second and third cycle) minutes in 400 (first cycle) or 200 (second and third cycle) μl of digestion mix (1% penicillin/streptomycin, 2% FBS, 100 mg/ml collagenase from Clostridium histolyticum, 100 mg/ml dispase II, 10 mg/ml deoxyribonuclease I from bovine pancreas in Iscove's Modified Dubecco's Medium). After each incubation, lymph nodes were homogenized by pipetting with a 1000 μl tip, supernatants were transferred to new tubes and kept at 4° C. while digestion mix was added to the remaining larger pieces and incubated at 37° C. Pooled supernatants of individual lymph nodes were divided into two aliquots: one for flow cytometry analysis, another one was centrifuged at 300×g for 5 minutes and the cell pellet was resuspended in 800 μl of TRI Reagent (Zymo Research, Irvine, CA, USA) for subsequent RNA isolation.

Flow cytometry. Single cell suspensions were stained using Zombie Red or Violet Fixable Viability Kit (BioLegend) in PBS for 5 minutes at 4° C., washed once with FACS buffer (0.2% BSA 0.05% NaN3 in PBS) and subsequently stained with antibodies against surface antigens diluted in FACS buffer for 20 minutes at 4° C., followed by two washing steps in FACS buffer. For fixation cells were incubated in Fixation Buffer (BioLegend. 420801) for 10 minutes at room temperature followed by a washing step in FACS buffer. Samples were acquired on a BD LSRFortessa (BD) flow cytometer and data were analyzed using FlowJo v.10 software (BD Biosciences).

The following antibodies were used: anti-CD45 BV510 (30-F11), anti-CD45 Alexa Fluor 700 (30-F11), anti-CD45 APC (30-F11), anti-CD45 PerCP/Cy5.5 (30-F11), anti-CD3 PE/Dazzle 594 (17A2), anti-CD3 BV510 (17A2), anti-CD4 PE/Cy5 (GK1.5), anti-CD4 APC/Cy5 (GK1.5), anti-CD19 PE/Dazzle 594 (6D5), anti-CD19 BV650 (6D5), anti-CD19 BV785 (6D5), anti-CD80 BV711 (16-10A1), anti-CD273 PE/Cy7 (TY25), anti-CD38 PE/Cy7 (90), anti-CD183 APC/Cy7 (CXCR3-173), anti-IgM BV711 (RMM-1), anti-IgM PerCP/Cy5.5 (RMM-1), anti-IgD PerCP/Cy5.5 (11-26c2a), anti-IgG PE/Cy7 (Poly4053), anti-IgG PE/Cy7 (Poly4053), anti-IgG APC/Cy7 (Poly4053), anti-CD185 (CXCR5) BV711 (L138D7), anti-278 PE/Cy7 (C398.4A), anti-GL7 Pacific Blue (GL7), anti-95 PE/Cy7 (Jo2), anti-Flag APC (L5). For spike-specific flow cytometry analysis SARS-CoV-2 spike protein (ECD, His & Flag tag) (GenScript Biotech, New Jersey, NJ, USA; Cat #Z03481), SARS-CoV-2 (BA.4/BA.5/BA.5.2) Spike S1+S2 trimer Protein (ECD, His tag) (SinoBiological, Beijing, China; Cat #40589-V08H32), SARS-CoV-2 XBB.1.5 (Omicron) Spike S1+S2 trimer Protein (ECD, His tag) (SinoBiological, Cat #40589-V08H45) were used. Alexa Fluor-488 Conjugation Kit (Fast)-Lightning-Link (Abcam, Cat #ab236553) and PE/R-Phycoerythrin Conjugation Kit (Fast)-Lightning-Link (Abcam, Cat #ab102918) were used to conjugate fluorescence tag in the spike protein according to the manufacturer's protocol. CountBright Absolute Counting Beads (Thermo Fisher Scientific, Cat #C36950) were used to quantify absolute cell numbers.

RNA isolation and sequencing. RNA was isolated from TRI Reagent (Zymo Research) samples using Direct-zol RNA Microprep and Miniprep kits (Zymo Research) according to the manufacturer's protocol. RNA library preparation with PolyA selection and Illumina Sequencing were conducted at Azenta Life Sciences (South Plainfield, NJ, USA) as follows: RNA samples were quantified using Qubit 2.0 Fluorometer (Life Technologies, Carlsbad, CA, USA) and RNA integrity was checked using Agilent TapeStation 4200 (Agilent Technologies, Palo Alto, CA, USA). Strand-specific RNA sequencing libraries were prepared by using NEBNext Ultra II Directional RNA Library Prep Kit for Illumina following manufacturer's instructions (NEB, Ipswich, MA, USA). Briefly, the enriched RNAs were fragmented for 8 minutes at 94° C. First strand and second strand cDNA were subsequently synthesized. The second strand of cDNA was marked by incorporating dUTP during the synthesis. cDNA fragments were adenylated at 3′ends, and indexed adapter was ligated to cDNA fragments.

Limited cycle PCR was used for library enrichment. The incorporated dUTP in second strand cDNA quenched the amplification of second strand, which helped to preserve the strand specificity. The sequencing library was validated on the Agilent TapeStation (Agilent Technologies), and quantified by using Qubit 2.0 Fluorometer (Thermo Fisher Scientific) as well as by quantitative PCR (KAPA Biosystems, Wilmington, MA, USA).

The sequencing libraries were clustered on the flowcell. After clustering, the flowcell was loaded on the Illumina instrument according to manufacturer's instructions. The samples were sequenced using a 2×150 bp Paired End configuration. Image analysis and base calling were conducted by the Control software. Raw sequence data (.bcl files) generated the sequencer were converted into fastq files and de-multiplexed using Illumina's bcl2fastq 2.20 software. One mismatch was allowed for index sequence identification.

FastQC-quality controlled reads were mapped to the mouse transcriptome (GRCm39) based on Ensembl annotations using Kallisto v0.50.1. Transcript counts were imported and aggregated to gene counts using tximport. Only genes passing a threshold of 6 or more counts in 4 or more samples were considered as expressed and further analyzed using the R package DESeq2. Differential expression tests were setup with an alternate hypothesis of an absolute log 2 FC greater than 0.5. Heatmaps were produced with the R package ComplexHeatmap, using Z-scored r log-normalized counts. Volcano plots were produced using the R package EnhancedVolcano, with an FDR-adjusted threshold of 0.01 and a log 2 fold change threshold of 1. Pathway analysis was performed with the R package fgsea, using the entire Broad Institute's MSigDB's Hallmark gene set collection and select signatures from Reactome, Gene Ontology Biological Process or publications, as indicated in the results section. Bubble plots were generated using the R package ggplot2, showing pathways passing a threshold of adjusted p-value of 0.05.

Splenocyte restimulation assay. To extract splenocytes, spleens were passed through a 70 μm cell strainer, rinsed with PBS and treated with 2 ml of Ammonium-chloride-potassium (ACK) lysis buffer for 2 minutes at room temperature to lyse red blood cells (RBC). After one more washing step with PBS, splenocytes were distributed into flat-bottom 96-well plates, with a density of 2×106 cells per well. SARS-CoV-2 Spike peptides (PepTivator SARS-CoV-2 Prot_S; Miltenyi Biotec, Bergisch Gladbach, Germany) were added to each well at a concentration of 0.6 nmol/ml, in a total volume of 200 μl per well. Supernatants were collected after 96 hours for the quantification of IFN-7 levels using the ELISA MAX Deluxe Set Mouse IFN-7 (BioLegend, Cat #430804), as per the instructions provided by the manufacturer.

Preparation of Candida albicans β-glucan particles. β-glucan was isolated from C. albicans SC5314 as previously described. Briefly, glucan was isolated from C. albicans using a base/acid extraction approach, which provides water insoluble glucan particles that are >95% pure. The structure and purity of the glucan was determined by ¹H-NMR in DMSO-d₆. Prior to use the β-glucan particles are depyrogenated and sterilized.

Isolation, differentiation, and stimulation of human phagocytes. Human peripheral blood mononuclear cells (PBMCs) were isolated or differentiated from collars of blood from healthy donors received from Boston Children's Hospital blood donation center. Blood was diluted 1:2 in PBS and PBMC were isolated using Histopaque (MilliporeSigma, Cat #1077-1) gradient centrifugation. Monocytes were positively selected from PBMCs using CD14 MicroBeads (Miltenyi Biotec, Cat #130-050-201) according to the manufacturer's instructions. PBMCs and CD14+ monocytes were further differentiated in RPMI 1640 medium (Thermo Fisher Scientific, Cat #61870127) supplemented with 10% FBS in presence of 10 ng/ml GM-CSF (BioLegend Cat #572903) (referred to as GM-CSF PBMC) for 16 hours or 20 ng/ml GM-CSF (BioLegend, Cat #572903) and 20 ng/ml IL-4 (BioLegend, Cat #574004) (referred to as moDC) for 7 days. PBMCs without GM-CSF treatment isolated at the same time were used as controls (referred to as PBMCs w/o GM-CSF). Prior to stimulation, cultured cells were detached with 2 mM EDTA in PBS and re-plated in flat bottom 96 well plates at a density of 10⁵ cells/well in 200 μl RPMI 1640 supplemented with 10% FBS, 2 mM L-glutamine, 1% penicillin/streptomycin and stimulated for 20-24 hours. If not otherwise indicated, cells were stimulated with 1 μg/ml LPS from E. coli, Serotype 055:B5 (TLRGRADE®) (Ready-to-Use) (LPS; Enzo Life Sciences, Farmingdale, NY, USA; cat #ALX-581-013-L001), β-Glucan (10 μg/ml), alum (4 μg/ml), mannans (10 μg/ml), the alum-mannan complex (10 μg/ml mannan together with 4 μg/ml alum), or saline as a vehicle control. Cell culture supernatants were collected to assess cytokines levels. To measure intracellular pro-IL-1β levels cells were lysed in PBS by freezing/thawing and the lysate was analyzed by ELISA.

Enzyme-linked immunosorbent assay (ELISA) for cytokines. Cytokine production was quantified from cell culture supernatants or lysates harvested after stimulation using the following kits according to the manufacturer's protocol: ELISA MAX Deluxe Set Human IL-1β (BioLegend, Cat #437004), ELISA MAX Deluxe Set Human IL-6 (BioLegend, Cat #430504), ELISA MAX Deluxe Set Human TNF-α (BioLegend, Cat #430204).

Western Blot. Western blot was performed using standard molecular biology techniques. Blots were probed using antibodies against: p-STAT1 (Tyr701) (Cell Signaling Technology, Danvers, MA, USA; cat #9167, clone 58D6), Viperin (Cell Signaling Technology, cat #13996, clone D5T2X), β-Actin (Proteintech, Rosemont, IL, USA; cat #20536-1-AP, polyclonal, Lot #00105345).

Statistical analysis. Data were tested for normality using the D'Agostino-Pearson test. If normality was not rejected at 0.05 significance level, data were analyzed using parametric tests. If normality was rejected and/or variances were different across groups, Log-transformation was done to meet the criteria of parametric tests. Statistical differences between 2 or more groups in datasets with one categorical variable were evaluated by One-way ANOVA with correction for multiple comparisons. Statistical differences between groups in datasets with two categorical variables were evaluated by Two-way ANOVA with correction for multiple comparisons.

Synthetic polymannose and Konjac glucomannan. Synthetic polymannose (20-30 kDa) was synthesized by first grinding D-mannose, sorbitol, and ascorbic acid (9:1:0.1 w/w) into a fine powder and mixing it thoroughly using a mortar and pestle. The powder was transferred to a beaker and placed in a vacuum oven. The powder was then heated at ~140° C. for 3 hours under vacuum to facilitate anhydrous melt polymerization. The product was cooled under ambient conditions and redissolved in water. Subsequently, 4 parts of ethanol was added, and the mixture was subjected to centrifugation (1,000-2,000 rpm; 10 minutes) to remove low molecular weight mannose. This extraction process was repeated three times. The lyophilized product was ground into a fine powder and stored until use. Konjac glucomannan (>99% purity, viscosity >30,000 mPas) was purchased as a dried powder extracted from the konjac plant.

Preparation of konjac glucomannan-containing FDC (FDCkgc) vaccines and alum-konjac glucomannan-containing (FDCakgc) vaccines. Alum-konjac glucomannan complex was prepared by incubated alum with konjac glucomannan (1:4 weight ratio alum:konjac glucomannan) in 0.9% saline for 30 minutes at room temperature. Subsequently, Comirnaty (BNT162b2 mRNA, Pfizer-BioNTech; 1 μg mRNA/mouse) was added to the alum-konjac glucomannan complex to form the alum-konjac glucomannan vaccine (FDCakgc). For the FDCkg vaccines, Comirnaty (1 μg mRNA/mouse) was added to 60 μg of Konjac glucomannan.

Analysis of lymph node responses. To assess lymph node innate responses, mice were intradermally injected on day 0 with the indicated compounds in a volume of 50 μl on each side of the back (one side for the compound and the contralateral side for saline). Draining (brachial) lymph nodes were collected 24 hours post-injection, or additionally 7 and 14 days post-injection where indicated, for analysis. Lymph nodes were weighed on an analytical scale prior to transfer to a tissue homogenizer and disrupted with beads in TRI reagent as indicated for skin samples, or processed to generate a lymph node cell suspension by a modified previously published protocol. Briefly, individual lymph nodes were incubated at 37° C. for 20 minutes in 400 μl of digestion mix (IMDM+pen/strep+2% FBS+100 mg/ml collagenase+100 mg/ml dispase II+10 mg/ml DNase). Then, lymph nodes were ground by pipetting with a 1000 μl tip, supernatants were transferred to new tubes and kept at 4° C. while 200 μl of digestion mix was added to the pellets and incubated at 37° C. for 10 minutes. This cycle was repeated one more time, then pooled supernatants of individual lymph nodes were divided into two aliquots: one for flow cytometry analysis, another one was centrifuged at 300×g for 5 minutes and the cell pellet was resuspended in 800 μl of TRI reagent for subsequent RNA isolation.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. An immunogenic composition comprising:
(i) an adjuvantation system comprising a mannan; and
(ii) a nucleic acid encoding an antigen.

2. The immunogenic composition of claim 1, wherein the mannan is a plant mannan or a fungal mannan.

3. The immunogenic composition of claim 2, wherein the mannan is a glucomannan, a galactomannan, or a galactoglucomannan.

4. The immunogenic composition of claim 2 or claim 3, wherein the mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan.

5. The immunogenic composition of any one of claims 2-4, wherein the plant mannan is a konjac glucomannan.

6. The immunogenic composition of claim 2, wherein the fungal mannan is a yeast mannan.

7. The immunogenic composition of claim 2, wherein the fungal mannan is a Candida albicans mannan.

8. The immunogenic composition of claim 1, wherein the adjuvantation system further comprises an aluminum salt.

9. The immunogenic composition of claim 8, wherein the aluminum salt is aluminum hydroxide, aluminum phosphate, or aluminum hydroxyphosphate.

10. The immunogenic composition of claim 8 or claim 9, wherein the weight ratio of aluminum salt to mannan in the adjuvantation system is between 1:1 and 1:100.

11. The immunogenic composition of any one of claims 8-10, wherein the weight ratio of aluminum salt to mannan in the adjuvantation system is 1:1, 1:2, 1:3, 1:4, 1:5, 1:10, or 1:100.

12. The immunogenic composition of any one of claims 1-11, wherein the nucleic acid is DNA or RNA.

13. The immunogenic composition of claim 12, wherein the RNA is a messenger RNA (mRNA).

14. The immunogenic composition of any one of claims 1-13, wherein the antigen is a bacterial antigen, a viral antigen, or a fungal antigen.

15. The immunogenic composition of claim 14, wherein the viral antigen comprises a Beta coronavirus protein or polypeptide.

16. The immunogenic composition of claim 15, wherein the Beta coronavirus protein or polypeptide comprises a Beta coronavirus spike protein or spike protein receptor binding domain (RBD).

17. The immunogenic composition of claim 16, wherein the Beta coronavirus spike protein is a MERS-CoV spike protein, a SARS-CoV-1 spike protein, or a SARS-CoV-2 spike protein.

18. The immunogenic composition of claim 16, wherein the Beta coronavirus spike protein RBD is a MERS-CoV spike protein RBD, a SARS-CoV-1 spike protein RBD, or a SARS-CoV-2 spike protein RBD.

19. The immunogenic composition of any one of claims 1-18, wherein the ratio of the nucleic acid to the adjuvantation system is between 1:1 and 1:100.

20. The immunogenic composition of any one of claims 1-19, wherein the ratio of the nucleic acid to the adjuvantation system is 1:1, 1:10, or 1:100.

21. The immunogenic composition of any one of claims 1-19, wherein the adjuvantation system and the nucleic acid are admixed.

22. An immunogenic composition comprising:
(i) an adjuvantation system comprising a plant mannan; and
(ii) an antigen.

23. The immunogenic composition of claim 22, wherein the plant mannan is a glucomannan, a galactomannan, or a galactoglucomannan.

24. The immunogenic composition of claim 22 or claim 23, wherein the plant mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan.

25. The immunogenic composition of any one of claims 22-24, wherein the plant mannan is a konjac glucomannan.

26. The immunogenic composition of any one of claims 22-25, wherein the adjuvantation system further comprises an aluminum salt.

27. The immunogenic composition of claim 26, wherein the aluminum salt is aluminum hydroxide, aluminum phosphate, or aluminum hydroxyphosphate.

28. The immunogenic composition of claim 26 or claim 27, wherein the weight ratio of aluminum salt to plant mannan in the adjuvantation system is between 1:1 and 1:100.

29. The immunogenic composition of any one of claims 26-28, wherein the weight ratio of aluminum salt to plant mannan in the adjuvantation system is 1:1, 1:2, 1:3, 1:4, 1:5, 1:10, or 1:100.

30. The immunogenic composition of any one of claims 22-29, wherein the antigen is a parasitic antigen, a bacterial antigen, a viral antigen, or a fungal antigen.

31. The immunogenic composition of claim 30, wherein the viral antigen comprises L1 antigen, E6 antigen, E7 antigen, HBsAg, Epstein-Barr nuclear antigen, SV40, or a Beta coronavirus protein or polypeptide.

32. The immunogenic composition of claim 30, wherein the viral antigen comprises a nucleic acid encoding comprises L1 antigen, E6 antigen, E7 antigen, HBsAg, Epstein-Barr nuclear antigen, SV40, or a Beta coronavirus protein or polypeptide.

33. The immunogenic composition of claim 32, wherein the nucleic acid is DNA or RNA.

34. The immunogenic composition of claim 33, wherein the RNA is a messenger RNA (mRNA).

35. The immunogenic composition of any one of claims 31-34, wherein the Beta coronavirus protein or polypeptide comprises a Beta coronavirus spike protein or spike protein receptor binding domain (RBD).

36. The immunogenic composition of claim 35, wherein the Beta coronavirus spike protein is a MERS-CoV spike protein, a SARS-CoV-1 spike protein, or a SARS-CoV-2 spike protein.

37. The immunogenic composition of claim 35, wherein the Beta coronavirus spike protein RBD is a MERS-CoV spike protein RBD, a SARS-CoV-1 spike protein RBD, or a SARS-CoV-2 spike protein RBD.

38. The immunogenic composition of any one of claims 22-37, wherein the adjuvantation system and the antigen are admixed.

39. A vaccine comprising the immunogenic composition of any one of claims 1-38 and a pharmaceutically acceptable excipient.

40. A nanoparticle comprising the immunogenic composition of any one of claims 1-38 or the vaccine of claim 39.

41. The nanoparticle of claim 40, wherein the nanoparticle is a lipid nanoparticle.

42. The nanoparticle of claim 40 or claim 41, wherein the nanoparticle has a diameter between 400 nm and 1600 nm.

43. A method of inducing an immune response against a pathogen in a subject in need thereof, the method comprising administering to the subject:
(i) an adjuvantation system comprising a mannan, and
(ii) a nucleic acid encoding an antigen.

44. The method of claim 43, wherein the mannan is a plant mannan or a fungal mannan.

45. The method of claim 43 or claim 44, wherein the mannan is a glucomannan, a galactomannan, or a galactoglucomannan.

46. The method of claim 44 or claim 45, wherein the plant mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan.

47. The method of any one of claims 44-46, wherein the plant mannan is a konjac glucomannan.

48. The method of claim 44 or claim 45, wherein the fungal mannan is a yeast mannan.

49. The method of claim 44 or claim 45, wherein the fungal mannan is a Candida albicans mannan.

50. The method of any one of claims 43-49, wherein the adjuvantation system further comprises an aluminum salt.

51. The method of any claim 50, wherein the aluminum salt is aluminum hydroxide, aluminum phosphate, or aluminum hydroxyphosphate.

52. The method of claim 50 or claim 51, wherein the weight ratio of aluminum salt to mannan is in the adjuvantation system between 1:1 and 1:100.

53. The method of any one of claims 50-52, wherein the weight ratio of aluminum salt to mannan in the adjuvantation system is 1:1, 1:2, 1:3, 1:4, 1:5, 1:10, or 1:100.

54. The method of any one of claims 43-53, wherein the nucleic acid is DNA or RNA.

55. The method of claim 54, wherein the RNA is a messenger RNA (mRNA).

56. The method of any one of claims 43-55, wherein the antigen is a bacterial antigen, a viral antigen, or a fungal antigen.

57. The method of claim 56, wherein the viral antigen comprises a Beta coronavirus protein or polypeptide.

58. The method of claim 57, wherein the Beta coronavirus protein or polypeptide comprises a Beta coronavirus spike protein or spike protein receptor binding domain (RBD).

59. The method of claim 58, wherein the Beta coronavirus spike protein is a MERS-CoV spike protein, a SARS-CoV-1 spike protein, or a SARS-CoV-2 spike protein.

60. The method of claim 58, wherein the Beta coronavirus spike protein RBD is a MERS-CoV spike protein RBD, a SARS-CoV-1 spike protein RBD, or a SARS-CoV-2 spike protein RBD.

61. The method of any one of claims 43-60, wherein the ratio of the nucleic acid to the adjuvantation system is between 1:1 and 1:100.

62. The method of any one of claims 43-61, wherein the ratio of the nucleic acid to the adjuvantation system is 1:1, 1:10, or 1:100.

63. The method of any one of claims 443-62, wherein the adjuvantation system and the nucleic acid are admixed.

64. The method of any one of claims 43-63, wherein the adjuvantation system increases recruitment of immune cells in the subject, compared to when the nucleic acid is administered alone.

65. The method of claim 64, wherein the immune cells are B cells or T cells.

66. A method of inducing an immune response against a pathogen in a subject in need thereof, the method comprising administering to the subject:
(i) an adjuvantation system comprising a plant mannan, and
(ii) an antigen.

67. The method of claim 66, wherein the plant mannan is a glucomannan, a galactomannan, or a galactoglucomannan.

68. The method of claim 66 or claim 67, wherein the plant mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan.

69. The method of any one of claims 66-68, wherein the plant mannan is a konjac glucomannan.

70. The method of any one of claims 66-69, wherein the adjuvantation system further comprises an aluminum salt.

71. The method of claim 70, wherein the aluminum salt is aluminum hydroxide, aluminum phosphate, or aluminum hydroxyphosphate.

72. The method of claim 70 or claim 71, wherein the weight ratio of aluminum salt to plant mannan is between 1:1 and 1:100.

73. The method of any one of claims 66-72, wherein the weight ratio of aluminum salt to plant mannan in the adjuvantation system is 1:1, 1:2, 1:3, 1:4, 1:5, 1:10, or 1:100.

74. The method of any one of claims 66-73, wherein the antigen is a bacterial antigen, a viral antigen, or a fungal antigen.

75. The method of claim 74, wherein the viral antigen comprises a Beta coronavirus protein or polypeptide.

76. The method of claim 74, wherein the viral antigen comprises a nucleic acid encoding a Beta coronavirus protein or polypeptide.

77. The method of claim 76, wherein the nucleic acid is DNA or RNA.

78. The method of claim 77, wherein the RNA is a messenger RNA (mRNA).

79. The method of any one of claims 75-78, wherein the Beta coronavirus protein or polypeptide comprises a Beta coronavirus spike protein or spike protein receptor binding domain (RBD).

80. The method of claim 79, wherein the Beta coronavirus spike protein is a MERS-CoV spike protein, a SARS-CoV-1 spike protein, or a SARS-CoV-2 spike protein.

81. The method of claim 79, wherein the Beta coronavirus spike protein RBD is a MERS-CoV spike protein RBD, a SARS-CoV-1 spike protein RBD, or a SARS-CoV-2 spike protein RBD.

82. The method of any one of claims 66-81, wherein the adjuvantation system and the antigen are admixed.

83. The method of any one of claims 66-82, wherein the adjuvantation system increases recruitment of immune cells in the subject, compared to when the antigen is administered alone.

84. The method of claim 83, wherein the immune cells are B cells or T cells.

85. The method of any one of claims 43-84, wherein the subject is a human.

86. The method of any one of claims 43-84, wherein the subject is a human neonate, a human infant, an adult human, or an elderly human.

87. The method of any one of claims 43-84, wherein the subject is a companion animal or a research animal.

88. The method of any one of claims 43-87, wherein the subject is immune-compromised, has chronic lung disease, asthma, cardiovascular disease, cancer, obesity, diabetes, chronic kidney disease, and/or liver disease.

89. The method of any one of claims 43-88, wherein the administration is intramuscular administration, intradermal administration, oral administration, intravenous administration, topical administration, intranasal administration, or sublingual administration.

90. The method of any one of claims 43-88, wherein the administration is intramuscular administration.

91. The method of any one of claims 43-90, wherein the administration is prophylactic.

92. An adjuvantation system comprising a plant mannan for use in inducing an immune response against a pathogen in a subject in need thereof.

93. The adjuvantation system of claim 92, wherein the mannan is a glucomannan, a galactomannan, or a galactoglucomannan.

94. The adjuvantation system of claim 92 or claim 93, wherein the mannan is konjac mannan, aloe vera mannan, salep mannan, porang mannan, ivory nut mannan, cassia gum mannan, locust bean gum mannan, guar gum mannan, tar gum mannan, fenugreek gum mannan, or Norway spruce mannan.

95. The adjuvantation system of any one of claims 92-94, wherein the mannan is a konjac glucomannan.

96. The adjuvantation system of any one of claims 92-95, wherein the adjuvantation system further comprises an aluminum salt.

97. The adjuvantation system of claim 96, wherein the aluminum salt is aluminum hydroxide, aluminum phosphate, or aluminum hydroxyphosphate.

98. The adjuvantation system of claim 96 or claim 97, wherein the weight ratio of aluminum salt to mannan in the adjuvantation system is between 1:1 and 1:100.

99. The adjuvantation system of any one of claims 96-98, wherein the weight ratio of aluminum salt to mannan in the adjuvantation system is 1:1, 1:2, 1:3, 1:4, 1:5, 1:10, or 1:100.


SEQUENCE LISTING (15 sequences)

SEQ ID NO: 1 - protein, 1255 aa, synthetic construct
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 222 aa, synthetic construct
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 3 - protein, 76 aa, synthetic construct
MYSFVSEETGTLIVNSVLLFLAFVVFLLVTLAILTALRLCAYCCNIVNVSLVKPTVYVYSRVKNLNSSEGVPDLLV

SEQ ID NO: 4 - protein, 221 aa, synthetic construct
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 5 - protein, 422 aa, synthetic construct
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 6 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 8 - protein, 75 aa, synthetic construct
MYSFVSEETGTLIVNSVLLFLAFVVFLLVTLAILTALRLCAYCCNIVNVSLVKPSFYVYSRVKNLNSSRVPDLLV

SEQ ID NO: 9 - protein, 222 aa, synthetic construct
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 10 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - protein, 1353 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSTVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 12 - protein, 122 aa, synthetic construct
ECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKC

SEQ ID NO: 13 - protein, 82 aa, synthetic construct
MLPFVQERIGLFIVNFFIFTVVCAITLLVCMAFLTATRLCVQCMTGFNTLLVQPALYLYNTGRSVYVKFQDSKPPLPPDEWV

SEQ ID NO: 14 - protein, 219 aa, synthetic construct
MSNMTQLTEAQIIAIIKDWNFAWSLIFLLITIVLQYGYPSRSMTVYVFKMFVLWLLWPSSMALSIFSAVYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 15 - protein, 413 aa, synthetic construct
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD